SEQUENCE LISTING (5 sequences)

SEQ ID NO: 1 - RNA, 50 nt, Artificial
  ribozyme
acagcacgccgcucugaugaguccgugaggacgaaaccacguucuucguc

SEQ ID NO: 2 - DNA, 20 bp, Artificial
  Primer for Scd1
ctacaagcctggcctcctgc

SEQ ID NO: 3 - DNA, 20 bp, Artificial
  Primer for Scd1
ggaccccagggaaaccagga

SEQ ID NO: 4 - DNA, 20 bp, Artificial
  Primer for ribosomal 18S
agggttcgattccggagagg

SEQ ID NO: 5 - DNA, 22 bp, Artificial
  Primer for ribosomal 18S
caactttaatatacgctattgg

(19) United States
(12) Patent Application Publication
(10) Pub. No.: US 2011/0207694 A1
(43) Pub. Date: Aug. 25, 2011

(54) MAMMALIAN HYPOTHALAMIC NUTRIENT MODULATION OF GLUCOSE METABOLISM

(76) Inventors: Luciano Rossetti, Bedford Hills, NY (US); Tony Lam, Richmond Hill (CA)

(21) Appl. No.: 13/026,851

(22) Filed: Feb. 14, 2011

Related U.S. Application Data
(63) Continuation of application No. 11/919,284, now abandoned.
(60) Provisional application No. 60/677,707, filed on May 3, 2005.
(60) Provisional application No. 60/677,708, filed on May 3, 2005.
(60) Provisional application No. 60/760,644, filed on Jan. 20, 2006.

(51) Int. Cl. A61K 31/715; A61K 31/19; A61K 31/191; A61K 31/7004; A61P 3/10; A61P 3/00; A61P 1/14

(52) U.S. Cl. 514/53; 514/557; 514/23; 514/54

(57) ABSTRACT

Provided are methods of reducing glucose production in a mammal, methods of reducing food intake in a mammal, methods of inhibiting gluconeogenesis in the liver of a mammal, methods of reducing peripheral blood glucose levels in a mammal, methods of decreasing serum triglyceride levels in a mammal, and methods of decreasing very low density lipoprotein (VLDL) levels in a mammal.

### CROSS-REFERENCE TO RELATED APPLICATION

This application claims the benefit of U.S. Provisional Patent Application Ser. No. 60/677,707, filed May 3, 2005, U.S. Provisional Patent Application Ser. No. 60/677,708, filed May 3, 2005, and U.S. Provisional Patent Application Ser. No. 60/760,644, filed Jan. 20, 2006, all incorporated by reference.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of DK 48321, DK 45024, DK 066058, and DK 47208 awarded by the National Institutes of Health.

### BACKGROUND OF THE INVENTION

(1) Field of the Invention

The present invention generally relates to regulation of glucose production in mammals.

More specifically, the invention relates to the modulation of hepatic glucose production and peripheral blood glucose levels through the increase in tricarboxylic acid (TCA) cycle flux through acetyl-CoA in the hypothalamus of the mammal.

(2) Description of the Related Art

### References Cited

Ahima, R. S., Prabakaran, D., Mantzoros, C., Qu, D., Maratos-Flier, E., & Flier, J. S. Role of leptin in the neuroendocrine response to fasting. Nature 382, 250-252 (1996).

Air, E. L., et al. Small molecule insulin mimetics reduce food intake and body weight and prevent development of obesity. Nat. Med. 8, 179-83 (2002).

Asilmaz, E. et al. Site and mechanism of leptin action in a rodent form of congenital lipodystrophy. J. Clin. Invest 113, 414-424 (2004).

Attie, A. D. et al. Relationship between stearoyl-CoA desaturase activity and plasma triglycerides in human and mouse hypertriglyceridemia. J. Lipid Res. 43, 1899-1907 (2002).

Bebernutz, G. R., et al. Anilides of (R)-trifluoro-2-hydroxy-2-methylpropionic acid inhibitors of pyruvate dehydrogenase kinase. J. Med. Chem. 43, 2248-2257 (2000).

Borg, M. A., Sherwin, R. S., Borg, W. P., Tamborlane, W. V. & Shulman, G. I. Local ventromedial hypothalamus glucose perfusion blocks counterregulation during systemic hypoglycemia in awake rats. J. Clin. Invest 99, 361-365 (1997).

Bruning, J. C., et al. Role of brain insulin receptor in control of body weight and reproduction. Science 289, 2122-25 (2000).

Cohen, P. et al. Role for stearoyl-CoA desaturase-1 in leptin-mediated weight loss. Science 297, 240-243 (2002).

Davis, J. D., Wirtshafter, D., Asin, K. E. & Brief, D. Sustained intracerebroventricular infusion of brain fuels reduces body weight and food intake in rats. Science 212, 81-83 (1981).

Frey, W. H. II. Bypassing the blood brain barrier to deliver therapeutic agents to the brain and spinal cord. Drug Delivery Tech. 2, 46-49 (2002).

Fisher, E. A. & Ginsberg, H. N. Complexity in the secretory pathway: the assembly and secretion of apolipoprotein B-containing lipoproteins. J. Biol. Chem. 277, 17377-17380 (2002).

Flier, J. S. Obesity wars: molecular progress confronts an expanding epidemic. Cell 116, 337-350 (2004).

Friedman, J. M. Obesity in the new millennium. Nature 404, 632-634 (2000).

Gill, J. M. et al. Hepatic production of VLDL1 but not VLDL2 is related to insulin resistance in normoglycaemic middle-aged subjects. Atherosclerosis 176, 49-56 (2004).

Ginsberg, H. N. New perspectives on atherogenesis: role of abnormal triglyceride-rich lipoprotein metabolism. Circulation 106, 2137-2142 (2002).

Gutierrez-Juarez, R. et al. Critical Role of Stearoyl-CoA Desaturase-1 (SCD1) in the Onset of Diet-induced Hepatic Insulin Resistance. J. Clin. Invest (In press) (2006).

Hill, J. O. & Peters, J. C. Environmental Contributions to Obesity. Science 280, 1371 (1998).

Hollenbeck, C. B. Dietary fructose effects on lipoprotein metabolism and risk for coronary artery disease. Am. J. Clin. Nutr. 58, 800S-809S (1993).

Horton, J. D., Goldstein, J. L. & Brown, M. S. SREBPs: activators of the complete program of cholesterol and fatty acid synthesis in the liver. J. Clin. Invest 109, 1125-1131 (2002).

Howard, J. K. et al. Enhanced leptin sensitivity and attenuation of diet-induced obesity in mice with haploinsufficiency of Socs3. Nat. Med. 10, 734-738 (2004).

Jackson, J. C., et al. Heterologously expressed inner lipoyl domain of dihydrolipoyl acetyltransferase inhibits ATP-dependent inactivation of pyruvate dehydrogenase complex. Biochem. J 334, 703-711 (1998).

Jiang, G. et al. Prevention of obesity in mice by antisense oligonucleotide inhibitors of stearoyl-CoA desaturase-1. J. Clin. Invest 115, 1030-1038 (2005).

Kalopissis, A. D., Griglio, S., Malewiak, M. I., Rozen, R. & Liepvre, X. L. Very-low-density-lipoprotein secretion by isolated hepatocytes of fat-fed rats. Biochem. J. 198, 373-377 (1981).

Kopelman, P. G. & Hitman, G. A. Diabetes. Exploding type II. Lancet 352, SIV5 (1998).

Kraus, W. E. et al. Effects of the amount and intensity of exercise on plasma lipoproteins. N. Engl. J. Med. 347, 1483-1492 (2002).

Lam, T. K., Schwartz, G. J. & Rossetti, L. Hypothalamic sensing of fatty acids. Nat. Neurosci. 8, 579-584 (2005a).

Lam, T. K., Gutierrez-Juarez, R., Pocai, A. & Rossetti, L. Regulation of blood glucose by hypothalamic pyruvate metabolism. Science 309, 943-947 (2005b).

Lam, T. K. et al. Hypothalamic sensing of circulating fatty acids is required for glucose homeostasis. Nat. Med. 11, 320-327 (2005c).

Levin, B. E., Routh, V. H., Kang, L., Sanders, N. M. & Dunn-Meynell, A. A. Neuronal glucosensing: what do we know after 50 years? Diabetes 53, 2521-2528 (2004).

Lewis, G. F., Uffelman, K. D., Szeto, L. W., Weller, B. & Steiner, G. Interaction between free fatty acids and insulin in the acute control of very low density lipoprotein production in humans. J. Clin. Invest 95, 158-166 (1995).

Lin, J. et al. Hyperlipidemic effects of dietary saturated fats mediated through PGC-lbeta coactivation of SREBP. Cell 120, 261-273 (2005).

Loftus, T. M., Jarkowsky, D. E., Frehynot, G. L., Towsend, C. A., Ronnet, G. V., Lane M. D., Kuhajda, F. P. Reduced Food Intake and Body Weight in Mice Treated with Fatty Acid Synthase Inhibitors. Science 288, 2379 (2000).

Mayers, R. M., et al. AZD7545, a novel inhibitor of pyruvate dehydrogenase kinase 2 (PDHK2), activates pyruvate dehydrogenase in vivo and improves blood glucose control in obese (fa/fa) Zucker rats. Biochem. Soc. Transactions 31, 1165-1167 (2003).

Miyazaki, M., Kim, Y. C., Gray-Keller, M. P., Attie, A. D. & Ntambi, J. M. The biosynthesis of hepatic cholesterol esters and triglycerides is impaired in mice with a disruption of the gene for stearoyl-CoA desaturase 1. J. Biol. Chem. 275, 30132-30138 (2000).

Morgan, K., Obici, S. & Rossetti, L. Hypothalamic responses to long-chain fatty acids are nutritionally regulated. J. Biol. Chem. 279, 31139-31148 (2004).

Muse, E. D. et al. Role of resistin in diet-induced hepatic insulin resistance. J. Clin. Invest 114, 232-239 (2004).

Obici, S. et al. Central melanocortin receptors regulate insulin action. J. Clin. Invest 108, 1079-1085 (2001).

Obici, S., Feng, Z., Morgan, K., Stein, D., Karkanias, G. & Rossetti, L. Central Administration of Oleic Acid Inhibits Glucose Production and Food Intake. Diabetes 51, 271-275 (2002a).

Obici, S., Zhang, B. B., Karkanias, G. & Rossetti, L. Hypothalamic insulin signaling is required for inhibition of glucose production. Nat. Med. 8, 1376-1382 (2002b).

Obici, S., Feng, Z., Arduini, A., Conti, R., and Rossetti, L. Inhibition of hypothalamic carnitine palmitoyltransferase-1 decreases food intake and glucose production. Nat. Med. 9, 756-761 (2003).

Otvos, J. D., Jeyarajah, E. J. & Bennett, D. W. Quantification of plasma lipoproteins by proton nuclear magnetic resonance spectroscopy. Clin. Chem. 37, 377-386 (1991)

Otvos, J. D., Jeyarajah, E. J., Bennett, D. W. & Krauss, R. M. Development of a proton nuclear magnetic resonance spectroscopic method for determining plasma lipoprotein concentrations and subspecies distributions from a single, rapid measurement. Clin. Chem. 38, 1632-1638 (1992).

Pan, M., Liang, J. S., Fisher, E. A. & Ginsberg, H. N. The late addition of core lipids to nascent apolipoprotein B 100, resulting in the assembly and secretion of triglyceride-rich lipoproteins, is independent of both microsomal triglyceride transfer protein activity and new triglyceride synthesis. J. Biol. Chem. 277, 4413-4421 (2002).

Pocai, A. et al. Hypothalamic KATP channels control hepatic glucose production. Nature 434, 1026-1031 (2005).

Pellerin, L. & Magistretti, P. J. Neuroscience. Let there be (NADH) light. Science 305, 50-52 (2004).

Reaven, G. M., Chen, Y. D., Jeppesen, J., Maheux, P. & Krauss, R. M. Insulin resistance and hyperinsulinemia in individuals with small, dense low density lipoprotein particles. J. Clin. Invest 92, 141-146 (1993).

Reilly, M. P. & Rader, D. J. The metabolic syndrome: more than the sum of its parts? Circulation 108, 1546-1551 (2003).

Schwartz, M. W., Woods, S. C., Porte, D. Jr., Seeley, R. J., Baskin, D. G. Central nervous system control of food intake. Nature 404, 661-671 (2000).

Schwartz, M. W. & Porte, D., Jr. Diabetes, obesity, and the brain. Science 307, 375-379 (2005).

Spanswick, D., Smith, M. A., Groppi, V. E., Logan, S. D. & Ashford, M. L. Leptin inhibits hypothalamic neurons by activation of ATP-sensitive potassium channels. Nature 390, 521-525 (1997).

Spanswick, D., Smith, M. A., Mirshamsi, S., Routh, V. H. & Ashford, M. L. Insulin activates ATP-sensitive K+ channels in hypothalamic neurons of lean, but not obese rats. Nat. Neurosci. 3, 757-758 (2000).

Taghibiglou, C. et al. Mechanisms of hepatic very low density lipoprotein overproduction in insulin resistance. Evidence for enhanced lipoprotein assembly, reduced intracellular ApoB degradation, and increased microsomal triglyceride transfer protein in a fructose-fed hamster model. J. Biol. Chem. 275, 8416-8425 (2000).

Verschoor, L., Chen, Y. D., Reaven, E. P. & Reaven, G. M. Glucose and fructose feeding lead to alterations in structure and function of very low density lipoproteins. Horm. Metab Res. 17, 285-288 (1985).

Vikramadithyan, R. K. et al. Human aldose reductase expression accelerates diabetic atherosclerosis in transgenic mice. J. Clin. Invest 115, 2434-2443 (2005).

Wang, J., Liu, R., Hawkins, M., Barzilai, N., Rossetti, L. A nutrient-sensing pathway regulates leptin gene expression in muscle and fat. Nature 393, 684-688 (1998).

Wolfrum, C., Asilmaz, E., Luca, E., Friedman, J. M. & Stoffel, M. Foxa2 regulates lipid metabolism and ketogenesis in the liver during fasting and in diabetes. Nature 432, 1027-1032 (2004).

Woods, S. C. & McKay, L. D. Intraventricular alloxan eliminates feeding elicited by 2-deoxyglucose. Science 202, 1209-1211 (1978).

Woods, S. C., Lotter, E. C., McKay, D. L., & Porte, D. Jr. Chronic intracerebroventricular infusion of insulin reduces food intake and body weight of baboons. Nature. 282, 503-5 (1979).

Wu, X., Zhou, M., Huang, L. S., Wetterau, J. & Ginsberg, H. N. Demonstration of a physical interaction between microsomal triglyceride transfer protein and apolipoprotein B during the assembly of ApoB-containing lipoproteins. J. Biol. Chem. 271, 10277-10281 (1996).

PCT Patent Application No. PCT/US2004/004344.

U.S. Provisional Patent Application No. 60/652,840.

Complex metabolic diseases such as obesity and type 2 diabetes mellitus are the result of multiple interactions between genes and environment (Hill & Peters, 1998; Kopelman & Hitman, 1998). Hypothalamic centers sense the availability of peripheral nutrients partly via redundant nutrient-induced peripheral signals such as leptin and insulin (Woods et al., 1979; Bruning et al., 2000; Friedman, 2000; Air et al., 2002; Schwartz et al., 2000; Ahima et al., 1996; Wang et al., 1998) and via direct metabolic signaling (Loftus et al., 2000; Obici et al., 2002a, 2003). In this regard, nutrient metabolism in selective hypothalamic neurons has been postulated to be a primary biochemical sensor for nutrient availability, which in turn exerts a negative feedback on endogenous (liver) and exogenous (food intake) nutrient fluxes (Obici et al., 2002a, 2003). It would be desirable to determine the nature of the relevant nutrient sensor. The present invention addresses that need.

### SUMMARY OF THE INVENTION

Accordingly, the inventors have discovered that treatments that increase tricarboxylic acid (TCA) cycle (TCA) flux through acetyl-CoA in the hypothalamus of a mammal causes a reduction in glucose production and peripheral blood glucose levels through an inhibition of gluconeogenesis. Serum triglyceride and VLDL levels also decrease with these treatments.

Thus, in some embodiments, the invention is directed to methods of reducing glucose production in a mammal. The methods comprise administering a compound to the mammal, where administering the compound to the mammal causes an increase in tricarboxylic acid (TCA) cycle flux through acetyl-CoA in the hypothalamus of the mammal and a reduction in glucose production in the mammal.

In other embodiments, the invention is directed to methods of reducing food intake in a mammal. The method comprise administering a compound to the mammal, where administering the compound to the mammal causes an increase in tricarboxylic acid (TCA) cycle flux through acetyl-CoA in the hypothalamus of the mammal and a reduction in food intake in the mammal.

The invention is additionally directed to methods of inhibiting gluconeogenesis in the liver of a mammal. The methods comprise administering a compound to the mammal, where administering the compound to the mammal causes an increase in tricarboxylic acid (TCA) cycle flux through acetyl-CoA in the hypothalamus of the mammal and an inhibition of gluconeogenesis in the mammal.

In further embodiments, the invention is directed to methods of reducing peripheral blood glucose levels in a mammal. The methods comprise administering a compound to the mammal, wherein administering the compound to the mammal causes an increase in tricarboxylic acid (TCA) cycle flux through acetyl-CoA in the hypothalamus of the mammal and a reduction in peripheral blood glucose levels in the mammal.

Additionally, the invention is directed to methods of decreasing serum triglyceride levels in a mammal. The methods comprise administering a compound to the mammal, where administering the compound to the mammal causes an increase in tricarboxylic acid (TCA) cycle flux through acetyl-CoA in the hypothalamus of the mammal and a decrease in serum triglyceride levels in the mammal.

The invention is further directed to methods of decreasing very low density lipoprotein (VLDL) levels in a mammal. The methods comprise administering a compound to the mammal, where administering the compound to the mammal causes an increase in tricarboxylic acid (TCA) cycle flux through acetyl-CoA in the hypothalamus of the mammal and a decrease in VLDL levels in the mammal.

In additional embodiments, the invention is directed to methods of increasing glucose production in a mammal. The methods comprise administering a compound to the mammal, where administering the compound to the mammal causes a decrease in tricarboxylic acid (TCA) cycle flux through acetyl-CoA in the hypothalamus of the mammal and an increase in glucose production in the mammal.

The invention is additionally directed to methods of increasing food intake in a mammal. The methods comprise administering a compound to the mammal, where administering the compound to the mammal causes a decrease in tricarboxylic acid (TCA) cycle flux through acetyl-CoA in the hypothalamus of the mammal and an increase in food intake in the mammal.

Additionally, the invention is directed to methods of decreasing very low density lipoprotein (VLDL) levels in a mammal. The methods comprise increasing long-chain fatty acyl-Co-A (LC-CoA) levels in the hypothalamus of the mammal in an amount effective to reduce VLDL levels in the mammal.

In additional embodiments, the invention is directed to methods of decreasing serum triglyceride levels in a mammal. The methods comprise increasing long-chain fatty acyl-Co-A (LC-CoA) levels in the hypothalamus of the mammal in an amount effective to decrease serum triglyceride levels in a mammal.

### BRIEF DESCRIPTION OF THE DRAWINGS

FIG. 1 is a diagram and graphs relating to the relationship of central glucose metabolism and glucose production. Panel (a) is a schematic diagram that illustrates carbohydrate metabolism in glial-neuronal cells. Panel (b) is graphs of experimental results showing that central D-glucose administration lowered circulating glucose levels, and this effect was inhibited by oxamate (OXA)(an inhibitor of lactate dehydrogenase) or the K_{ATP} blocker glibenclamide (GLI)(see U.S. Provisional Patent Application 60/652,840, incorporated by reference). During pancreatic basal insulin clamps, central glucose administration increased exogenous glucose infusion rate required to maintain euglycemia. Panel (c) is graphs of experimental results showing that central glucose reduced liver glucose production, G6Pase flux and mRNA. These effects were inhibited by co-infusion of OXA or GLI. Panel (d) is graphs of experimental results showing that central glucose inhibited gluconeogenesis and glycogenolysis. These effects were prevented by co-infusion of OXA or GLI.

FIG. 2 is graphs relating to the relationship of central lactate levels and glucose production. Panel (a) is graphs of experimental results showing that central L-lactate administration lowered circulating glucose levels, and this effect was inhibited by OXA or GLI. During the pancreatic basal insulin clamps, central lactate administration increased exogenous glucose infusion rate required to maintain euglycemia. Panel (b) is graphs of experimental results showing that central lactate reduced liver glucose production, G6Pase flux and mRNA. Panel (c) is graphs of experimental results showing that central lactate inhibited gluconeogenesis and glycogenolysis, and these effects were prevented by co-infusion of OXA or GLI.

FIG. 3 is a diagram of experimental protocols (Panel (a)) and graphs of results of those experiments utilizing intrahypothalamic (IH) infusions of glucose. Panel (b) shows that IH glucose administration lowered circulating glucose levels, and this effect was prevented by K_{ATP} blocker GLI. Panel (c) shows that IH glucose administration increased exogenous glucose infusion rate to maintain euglycemia during the pancreatic basal insulin clamps, and this effect was prevented by GLI. Panel (d) shows that glucose utilization was unchanged by these treatments. Panels (e) and (f) show that IH glucose administration suppressed liver glucose production.

FIG. 4 is a schematic and graphs of experimental results showing the effect of dichloroacetate (DCA), an inhibitor of pyruvate dehydrogenase kinase, having the effect of activating pyruvate dehydrogenase (PDH). The schematic of Panel (a) illustrates the PDH activation effect of DCA in the context of glucose metabolism, the TCA cycle, and nervous system cells. Panel (b) shows that IH DCA administration lowered circulating glucose levels. Panel (c) shows that, during pancreatic basal insulin clamps, IH DCA increased exogenous glucose infusion rate to maintain euglycemia. Also, IH DCA did not affect glucose utilization, but decreased glucose production.

FIG. 5 is graphs of experimental results showing that, during pancreatic clamps, intravenous infusion of lactate (which elevates plasma lactate ˜3-fold) did not affect glucose infusion kinetics. In contrast, when ICV LDH inhibitor OXA or K_{ATP} blocker GLI was infused, IV lactate led to decreased glucose infusion rate to maintain euglycemia. More importantly, this was due to an increase in glucose production and not decrease in glucose utilization.

FIG. 6 is graphs of experimental results showing that central administration of glucose lowered glucose production in regular chow, but not 3-day high fat diet-fed animals. In contrast, central administration of lactate lowered glucose production in both regular chow and 3-day high fat diet-fed animals.

FIG. 7 is graphs of experimental results showing that central administration of glucose or lactate inhibits food intake overnight. This effect was prevented by co-administration of the LDH inhibitor OXA.

FIG. 8 is graphs of experimental results and a schematic of metabolic pathways. Panel a is a schematic representation of a hypothesis. In that hypothesis, the stimulation of brain glucose metabolism enhances the conversion of lactate to pyruvate via lactate dehydrogenase (LDH) and activates K_{ATP} channels. The latter in turn leads to inhibition of VLDL secretion and lowering of plasma triglycerides. Panel b shows that the central administration of glucose (black bars; n=5) or L-lactate (gray bars; n=5) lowers plasma triglyceride levels compared with mannitol (left clear bars; n=4) or D-lactate (right clear bars: n=5) during basal and pancreatic basal-insulin clamp (clamp) conditions. Plasma free fatty acids (FFA) were unchanged. *P<0.01 vs. the corresponding controls. Panel c shows that the ICV co-infusion of the LDH inhibitor (oxamate; left gray bars; n=4) or K_{ATP} channels blocker (glibenclamide; right gray bars; n=5) abolishes the plasma triglyceride-lowering effects of glucose (black bars; n=5). LDH inhibitor (left clear bars; n=4) or _{KATP} channels blocker (right clear bars; n=4) per se did not modify triglyceride levels. Plasma free fatty acids (FFA) were unchanged. *P<0.01 vs. the corresponding controls. Panel d shows that the ICV co-infusion of the LDH inhibitor (oxamate; left gray bars; n=4) or K_{ATP}, channel blocker (glibenclamide; right gray bars; n=5) abolished the plasma triglyceride-lowering effects of lactate (black bars; n=5). LDH inhibitor (left clear bars; n=4) or K_{ATP} channels blocker (right clear bars; n=4) per se did not alter plasma triglycerides. Plasma free fatty acids (FFA) were unchanged. *P<0.01 vs. the corresponding controls. Panel e shows that the central administration of glucose (black square; n=4) decreases plasma triglyceride levels in a time-dependent fashion compared to mannitol (clear square; n=4) under basal conditions. *P<0.01 vs. the corresponding controls. Values are means±SEM. The values during the basal and clamp represent steady-state levels obtained by averaging the result of at least five plasma samples during the experimental.

FIG. 9 is graphs of experimental results showing the effect of central glucose on VLDL secretion. In panel a, the intravenous administration of the lipoprotein lipase inhibitor tyloxapol was combined with ICV infusions of vehicle, glucose, or glucose and LDH or K_{ATP} channel inhibitors. ICV glucose (black squares and bars; n=6) decreased the rate of appearance of VLDL-triglycerides in plasma compared with vehicle (clear squares and bars; n=5). The ICV co-infusion of LDH inhibitor (left gray squares and bars; n=4) and K_{ATP} channels blocker (right gray squares and bars; n=7) abolished the effect of ICV glucose. *P<0.001 vs. all groups. Panel b shows that ICV glucose (black bars; n=6) increases liver triglyceride content but lowers plasma triglyceride levels compared with vehicle (clear bars; n=5). ICV co-infusion of the LDH inhibitor (left gray bars; n=4) and K_{ATP} channels blocker (right gray bars; n=7) reversed these effects of ICV glucose. *P<0.01 vs. all groups. Panel c shows that ICV glucose (black bars; n=6) lowers the average size of plasma VLDL particles compared with vehicle (clear bars; n=5). This effect was negated by the ICV co-infusion of LDH inhibitor (left gray bars; n=4) or K_{ATP} channels blocker (right gray bars; n=7). *P<0.05 vs. all groups. Panel d shows that the ICV glucose (black squares and bars; n=6) decreases the rate of large triglyceride-rich VLDL particles production compared with vehicle (clear square and bars; n=5). This effect was negated by the ICV co-infusion of LDH inhibitor (left gray square and bars; n=4) or K_{ATP} channels blocker (right gray square and bars; n=7). *P<0.05 vs. all groups. Panel e shows that ICV glucose (black squares and bars; n=4) fails to decrease plasma VLDL-triglycerides in hepatic vagotomized rats (gray squares and bars; n=5). Hepatic branch vagotomy alone (clear squares and bars with dotted line; n=5) had no effects on VLDL-triglycerides vs. vehicle (clear squares and bars; n=4). *P<0.01 vs. all groups.

FIG. 10 is a diagram, photographs of western blots, and graphs of experimental results showing that brain glucose metabolism inhibits hepatic SCD1 activity. Panel a shows a simplified scheme of the biochemical steps of hepatic VLDL synthesis and assembly. Triglyceride synthesis depends on the esterification of plasma FFA as well as on de novo lipogenesis, requiring the activity of acetyl-CoA carboxlyase (ACC) and fatty acid synthase (FAS). Apolipoprotein B48 (ApoB 48) provides the protein core for the nascent VLDL formation. Microsomal triglyceride transfer protein (MTP) plays a pivotal role in VLDL maturation, and oleyl-CoA regulates the final steps of VLDL assembly. SCD1 catalyzes the formation of oleyl-CoA from stearoyl-CoA. Panel b shows western blots of phospho-ACC, ACC, FAS, MTP and ApoB. Stimulation of brain glucose metabolism did not decrease the liver expression of those proteins. Panel c shows that ICV glucose (black bars; n=4) increases hepatic stearoyl-CoA (18:0) and decreases oleyl-CoA (18:1) levels compared with vehicle (clear bars; n=4). These effects were negated by the ICY co-infusion of LDH inhibitor (left gray bars; n=4) and K_{ATP} channels blocker (right gray bars; n=4). *P<0.05 vs. all groups. The hepatic desaturation index (ratio of 18:1/18:0) was decreased by ICV glucose (black bars; n=4) and this effect required the central activity of LDH (left gray bars; n=4) and K_{ATP} channels (right gray bars; n=4). *P<0.05 vs. all groups. Panel d shows that ICY glucose (black bars; n=4) decreases hepatic SCD1 mRNA and activity compared with vehicle (clear bars; n=4). These effects were abolished by the ICV co-infusion of LDH inhibitor (left gray bars; n=4) and K_{ATP} channels blocker (right gray bars; n=4). *P<0.05 vs. all groups. Panel e shows that central administration of glucose for only 60 min (black bars; n=4) decreases liver oleyl-CoA levels, desaturation index and SCD1 activity compared to mannitol (clear bar; n=4). *P<0.01 vs. vehicle.

FIG. 11 is diagrams and graphs of experimental results showing the effect of hepatic SCD1 activity and oleyl-CoA on VLDL secretion. Panel a is a diagram showing that all rats received two intra-peritoneal injections of SCD1 or scrambled (SCR) ASO prior to the lipoprotein studies. This protocol led to marked inhibition of SCD1 activity in the liver. Panel b shows that a decrease in the hepatic activity of SCD1 (black bar; n=5) decreases the rate of appearance of VLDL-triglycerides in plasma compared with SCR (clear bar; n=5). *P<0.001 vs. SCR. In Panel c, all rats received one portal injection of SCD1 or SCR ASO prior to the lipoprotein studies. This protocol led to marked inhibition of SCD1 activity selective in the liver but not in other tissues. Portal SCD1 ASO (black bar; n=4) decreased liver oleyl-CoA levels and hepatic TG-VLDL secretion rate. More importantly, portal oleate infusion normalized liver oleyl-CoA levels and TG-VLDL secretion in portal SCD1 ASO (gray bar; n=4). *P<0.01 vs. all groups. Panel d shows that ICV glucose (black squares and bar; n=4) fails to inhibit TG-VLDL secretion rate and liver oleyl-CoA levels during oleate infusion replacement (gray squares and bar; n=5). It is important to note that ICV glucose was infused for 2 h prior to the secretion studies. *P<0.01 vs. all groups.

FIG. 12 is a diagram and graphs of experimental results showing that brain glucose but not lactate fails to inhibit plasma triglyceride levels and hepatic VLDL secretion in overfed rats. Panel a shows the experimental protocol for both the clamp and the tyloxapol studies. Panel b shows that high fat feeding (HF, black bars; n=4) increases plasma triglyceride levels compared to standard chow rats (SC, clear bars; n=4). ICV glucose (left gray bars; n=4) failed to decrease plasma triglycerides, but ICV lactate (right gray bars with dotted line; n=6) lowered plasma triglycerides in HF rats to levels similar to those of SC rats (clear bar). *P<0.01 vs. SC and HF with ICV Lactate. Panel c the results when intravenous administration of the lipoprotein lipase inhibitor tyloxapol is combined with ICV infusions of vehicle, glucose, or lactate in HF rats. ICV glucose (left gray bars; n=4) failed to decrease the rate of appearance of VLDL-triglycerides in plasma compared with vehicle (black bars; n=5). On the other hand, ICY lactate (right gray bars with dotted line; n=4) decreased the rate of appearance of VLDL-triglycerides in plasma to levels similar to those of SC rats.

FIG. 13 is a diagram and graphs of experimental results showing that hypothalamic sensing of circulating lactate is required to inhibit hepatic TG-VLDL secretion. Panel a shows that circulating lactate (black bar and squares; n=5) lowers liver oleyl-CoA levels, desaturation index and SCD1 activity. *P<0.05 vs. vehicle. Panel b shows a schematic representation of hypothesis. Metabolism of lactate to pyruvate within the mediobasal hypothalamus (MBH) is required for the ability of circulating lactate to inhibit plasma TG levels and hepatic TG-VLDL secretion. Oxamate is the competitive inhibitor of lactate dehydrogenase (LDH). Panel c shows that circulating lactate (black bar; n=6) lowers plasma triglyceride during basal and pancreatic euglycemic-basal insulin clamp conditions compared to saline (clear bar; n=6). MBH LDH inhibitor (gray bar; n=5) abolished the ability of circulating lactate to inhibit plasma TG levels. *P<0.01 vs. all groups. The values during the basal and clamp represent steady-state levels obtained by averaging the result of at least five plasma samples during the experimental. Panel d shows the protocol for the TG-VLDL secretion studies during circulating lactate infusion (elevated plasma lactate level to ˜2 fold) in the presence (gray bar and squares; n=6) or absence (black bar and squares; n=5) of MBH LDH inhibitor. *P<0.001 vs. saline infusion. Panel e shows that circulating lactate (black bar and squares; n=5) fails to inhibit TG-VLDL secretion in the presence of MBH LDH inhibitor (gray bar and squares; n=6). *P<0.05 vs. all groups.

FIG. 14 is graphs of experimental results showing that the rate of appearance of total (small+medium+large) VLDL particles was not different among experimental groups in experiments described in Example 3.

### DETAILED DESCRIPTION OF THE INVENTION

The present invention is based in part on the inventors' identification of a hypothalamic nutrient signal that affects glucose production, peripheral blood glucose levels, food intake, VLDL levels, and triglyceride levels. The inventors have also characterized this signal and discovered ways to manipulate the signal to lower mammalian glucose production, peripheral blood glucose levels, food intake, and VLDL and triglyceride levels, or to raise glucose production, or food intake.

Without being bound by any particular mechanism for these effects, the inventors' data (see Example 1) leads the skilled artisan to conclude that the hypothalamic nutrient signal resides in the rate of tricarboxylic acid (TCA) cycle flux through acetyl-CoA in the hypothalamus. This conclusion is based in part on the ability to lower glucose production and peripheral glucose levels by any of several methods experimentally employed that has the effect of increasing TCA cycle flux through acetyl-CoA. These include (but are not limited to) adding lactate, pyruvate or glucose to the hypothalamus, or utilizing pharmacological agents that affect TCA cycle flux. TCA cycle flux through acetyl-CoA is increased by increasing anaplerotic or decreasing cataplerotic reactions of the TCA cycle.

Thus, in some embodiments, the invention is directed to methods of reducing glucose production in a mammal. The methods comprise administering a compound to the mammal, where administering the compound to the mammal causes an increase in tricarboxylic acid (TCA) cycle flux through acetyl-CoA in the hypothalamus of the mammal and a reduction in glucose production in the mammal.

In these embodiments, glucose production can be determined, e.g., as described in the Examples, or in PCT Patent Application No. PCT/US2004/004344 or U.S. Provisional Application 60/652,840, both incorporated by reference. Alternatively, reduced glucose production can be inferred through the measurement of lowered peripheral blood glucose levels, since glucose production is established herein to be the cause of a reduction in peripheral blood glucose levels in these methods.

Preferably, the compound is administered intranasally. The compositions and methods of the present invention provide for the delivery of compounds to the hypothalamus by the nasal route, while minimizing systemic exposure. In this regard and without being bound to any particular theory, it is believed that targeting the CNS by nasal administration is based on capture and internalization of substances by the olfactory receptor neurons, which substances are then transported inside the neuron to the olfactory bulb of the brain (Frey, 2002). Olfactory receptor neurons from the lateral olfactory tract within the olfactory bulb project to various regions such as the hippocampus, amygdala, thalamus, hypothalamus and other regions of the brain that are not directly involved in olfaction. These substances may also pass through junctions in the olfactory epithelium at the olfactory bulb and enter the subarachnoid space, which surrounds the brain, and the cerebral spinal fluid (CSF), which bathes the brain. Either pathway allows for targeted delivery without interference by the blood brain barrier, as neurons and the CSF, not the circulatory system, are involved in these transport mechanisms. Accordingly, intranasal delivery pathways permit compartmentalized delivery of compositions with substantially reduced systemic exposure and the resulting side effects.

In particular embodiments, the pharmaceutical composition is delivered to the olfactory region and/or the sinus region of the nose. The olfactory region is a small area that is typically about 2-10 cm² in man located in the upper third of the nasal cavity for deposition and absorption by the olfactory epithelium and subsequent transport by olfactory receptor neurons. Located on the roof of the nasal cavity, the olfactory region is desirable for delivery because it is the only known part of the body in which an extension of the CNS comes into contact with the environment (Bois et al., Fundamentals of Otolaryngology, p. 184, W. B. Saunders Co., Phila., 1989).

As further advantages, nasal delivery offers a noninvasive means of administration that is safe and convenient for self-medication, and which reduces the first-pass hepatic effect (i.e., metabolic degradation by the liver), which can result in greater bioavailability and lower dosages of the therapeutic agent. Intranasal administration can also provide for rapid onset of action due to rapid absorption by the nasal mucosa. These characteristics of nasal delivery result from several factors, including: (1) the nasal cavity has a relatively large surface area of about 150 cm² in man, (2) the submucosa of the lateral wall of the nasal cavity is richly supplied with vasculature, and (3) the nasal epithelium provides for a relatively high drug permeation capability due to thin single cellular layer absorption.

In preferred embodiments of these methods, the mammal has a condition that would likely be at least partially alleviated from reduced glucose production. As used herein, partial alleviation of a condition is achieved if at least a small proportion of a group of mammals having the condition and subjected to the invention methods experience at least a partial reduction in the symptoms or progression of the condition as a result of the methods. Nonlimiting examples of such conditions include obesity, type 2 diabetes, type 1 diabetes, leptin resistance, metabolic syndrome, insulin resistance, gonadotropin deficiency, amenorrhea, lactic acidosis (including congenital lactic acidosis), and polycystic ovary syndrome.

One way to cause an increase in TCA cycle flux through acetyl-CoA in the hypothalamus is to add pyruvate or a metabolic precursor of pyruvate to the hypothalamus. See Example 1. Examples of such compounds include lactate, glucose or another monosaccharide, a disaccharide such as sucrose, or an oligosaccharide. In some preferred embodiments, the compound is lactate. In other preferred embodiments, the compound is glucose.

Another way to cause an increase in TCA cycle flux through acetyl-CoA in the hypothalamus is to add a compound to the hypothalamus, where the compound increases the activity of an acetyl-CoA-increasing molecule in the hypothalamus of the mammal. As used herein, an acetyl-CoA-increasing molecule is a molecule affecting carbohydrate/TCA cycle metabolism that has the effect of promoting production of acetyl-CoA, thus causing an increase in TCA cycle flux through acetyl-CoA. Included are enzymes or carrier proteins, now known or later discovered, that drive carbohydrate/TCA cycle metabolism toward production of acetyl-CoA. Non-limiting examples of enzymes included within these embodiments are lactate dehydrogenase (LDH), pyruvate dehydrogenase (PDH), pyruvate dehydrogenase phosphatase, and glucose-6-phosphatase.

As used herein, “increasing the activity” encompasses methods that increase the action of a preexisting molecule as it relates to carbohydrate/TCA cycle metabolism, or increasing the amount of such molecules, or combinations thereof. The amount of a molecule can be increased by reducing the rate of degradation or removal of the molecule and/or increasing the biosynthesis of the molecule and/or adding the molecule. Conversely, “decreasing the activity” of a molecule means either reducing the action (e.g., enzyme activity) of a preexisting molecule as it relates to carbohydrate/TCA cycle metabolism, or reducing the amount of such molecules, or combinations thereof. It should be understood that the amount of the molecules can be reduced by increasing the rate of degradation or removal of the molecule and/or reducing the biosynthesis of the molecule.

The compound activating the acetyl-CoA-increasing molecule in these embodiments can be a small organic molecule (e.g., less than 2000 mw) that binds and activates the acetyl-CoA-increasing molecule. An example is a pyruvate dehydrogenase activator such as alpha-lipoic acids, e.g., dexlipotam. Alternatively, the compound can be a nucleic acid, such as an aptamer, that binds and activates the acetyl-CoA-increasing molecule, or a vector that comprises a sequence that encodes the acetyl-CoA-increasing molecule. Nucleic acid mimetics, as are known in the art, are also included as within the scope of these embodiments. In other embodiments, the compounds can be a protein, e.g., the acetyl-CoA-increasing molecule itself. These proteins can include peptidomimetics replacing any number of the constituent amino acids, by methods known in the art.

In some preferred embodiments, the acetyl-CoA-increasing molecule is a pyruvate dehydrogenase. In other preferred embodiments, the acetyl-CoA-increasing molecule is a lactate dehydrogenase.

In other embodiments of these methods, the compound decreases the activity of an acetyl-CoA-decreasing molecule in the hypothalamus of the mammal.

As used herein, an acetyl-CoA-decreasing molecule is a molecule affecting carbohydrate/TCA cycle metabolism that has the effect of inhibiting production of acetyl-CoA, thus causing a decrease in TCA cycle flux through acetyl-CoA. Included are enzymes or carrier proteins, now known or later discovered, that inhibit carbohydrate/TCA cycle metabolism toward production of acetyl-CoA. A non-limiting example of an enzyme included within these embodiments is pyruvate dehydrogenase kinase (PDHK).

Also included as acetyl-CoA-decreasing molecules are short interfering RNAs (siRNAs) or other molecules (e.g., cytokines and inhibitors) that directly or indirectly inhibit production of enzymes that promote production or buildup of LC-CoA. As is well known, siRNAs, cytokines, inhibitors and other molecules can have a strong effect in regulating the production or activity of an enzyme or carrier protein.

In some preferred embodiments, the acetyl-CoA-decreasing molecule is PDHK.

The compound that decreases the acetyl-CoA-decreasing molecule can be a small organic molecule, a nucleic acid or mimetic (including but not limited to aptamers, ribozymes, or antisense molecules), or oligopeptides or proteins.

Where the acetyl-CoA-decreasing molecule is PDHK, the compound is preferably a small organic molecule inhibitor of PDHK, such as dichloroacetic acid, chlorofluoroacetic acid, difluoroacetic acid, AZD7545 (Mayers et al., 2003), an anilide derivative of (R)-3,3,3-trifluoro-2-hydroxy-2-methylpropionic acid (Bebernitz et al., 2000), or a secondary amine of (R)-3,3,3-trifluoro-2-hydroxy-2-methylpropionic acid. Another PDHK inhibitor is an inner lipoyl domain of dihydrolipoyl acetyltransferase (Jackson et al., 1998).

In these embodiments, the compound can be administered directly to the brain of the mammal, e.g., by direct injection (see Example) or through a pump. Alternatively, the compound is administered peripherally in a manner that permits the activator to cross the blood-brain barrier of the mammal sufficiently to increase TCA cycle flux through acetyl-CoA in the hypothalamus, for example by intranasal administration. With any mode of administration, the compound is preferably formulated in a pharmaceutically acceptable excipient.

By “pharmaceutically acceptable” it is meant a material that (i) is compatible with the other ingredients of the composition without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers include, without limitation, any of the standard pharmaceutical carriers such as phosphate buffered saline solutions, water, emulsions such as oil/water emulsions, microemulsions, and the like.

The above-described compounds can be formulated without undue experimentation for administration to a mammal, including humans, as appropriate for the particular application. Additionally, proper dosages of the compositions can be determined without undue experimentation using standard dose-response protocols.

Accordingly, the compositions designed for oral, nasal, lingual, sublingual, buccal and intrabuccal administration can be made without undue experimentation by means well known in the art, for example with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, cornstarch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

The compounds can easily be administered parenterally such as for example, by intravenous, intramuscular, intrathecal or subcutaneous injection. Parenteral administration can be accomplished by incorporating the compounds into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the compound, in a pharmaceutical composition, into the rectum or large intestine. This can be accomplished using suppositories or enemas. Suppository formulations can easily be made by methods known in the art. For example, suppository formulations can be prepared by heating glycerin to about 120° C., dissolving the composition in the glycerin, mixing the heated glycerin after which purified water may be added, and pouring the hot mixture into a suppository mold.

Transdermal administration includes percutaneous absorption of the composition through the skin. Transdermal formulations include patches (such as the well-known nicotine patch), ointments, creams, gels, salves and the like.

Where the compound is administered peripherally such that it must cross the blood-brain barrier, the compound is preferably formulated in a pharmaceutical composition that enhances the ability of the compound to cross the blood-brain barrier of the mammal. Such formulations are known in the art and include lipophilic compounds to promote absorption. Uptake of non-lipophilic compounds can be enhanced by combination with a lipophilic substance. Lipophilic substances that can enhance delivery of the compound across the nasal mucus include but are not limited to fatty acids (e.g., palmitic acid), gangliosides (e.g., GM-1), phospholipids (e.g., phosphatidylserine), and emulsifiers (e.g., polysorbate 80), bile salts such as sodium deoxycholate, and detergent-like substances including, for example, polysorbate 80 such as Tween™, octoxynol such as Triton™ X-100, and sodium tauro-24,25-dihydrofusidate (STDHF). See Lee et al., Biopharm., April 1988 issue:3037.

In particular embodiments of the invention, the compound is combined with micelles comprised of lipophilic substances. Such micelles can modify the permeability of the nasal membrane to enhance absorption of the compound. Suitable lipophilic micelles include without limitation gangliosides (e.g., GM-1 ganglioside), and phospholipids (e.g., phosphatidylserine). Bile salts and their derivatives and detergent-like substances can also be included in the micelle formulation. The compound can be combined with one or several types of micelles, and can further be contained within the micelles or associated with their surface.

Alternatively, the compound can be combined with liposomes (lipid vesicles) to enhance absorption. The compound can be contained or dissolved within the liposome and/or associated with its surface. Suitable liposomes include phospholipids (e.g., phosphatidylserine) and/or gangliosides (e.g., GM-1). For methods to make phospholipid vesicles, see for example, U.S. Pat. No. 4,921,706 to Roberts et al., and U.S. Pat. No. 4,895,452 to Yiournas et al. Bile salts and their derivatives and detergent-like substances can also be included in the liposome formulation.

The compositions of the invention can be formulated for intranasal administration in a pharmaceutical carrier in accordance with known techniques. See, e.g., Remington, The Science And Practice of Pharmacy (20^{th} edition, 2000). Suitable nontoxic pharmaceutically acceptable nasal carriers will be apparent to those skilled in the art of nasal pharmaceutical formulations (see, e.g., Remington's Pharmaceutical Sciences (Maack Publishing Co., Easton latest edition). Further, it will be understood by those skilled in the art that the choice of suitable carriers, absorption enhancers, humectants, adhesives, etc., will typically depend on the nature of the active compound and the particular nasal formulation, for example, a nasal solution (e.g., for use as drops, spray or aerosol), a nasal suspension, a nasal ointment, a nasal gel, or another nasal formulation.

The carrier can be a solid or a liquid, or both, and is optionally formulated with the composition as a unit-dose formulation. Such dosage forms can be powders, solutions, suspensions, emulsions and/or gels. With respect to solutions or suspensions, dosage forms can be comprised of micelles of lipophilic substances, liposomes (phospholipid vesicles/membranes), and/or a fatty acid (e.g., palmitic acid). In particular embodiments, the pharmaceutical composition is a solution or suspension that is capable of dissolving in the fluid secreted by mucous membranes of the olfactory epithelium, which can advantageously enhance absorption.

In particular embodiments, the compound is one that is at least partially, or even substantially (e.g., at least 80%, 90%, 95% or more), soluble in the fluids that are secreted by the nasal mucosa (e.g., the mucosal membranes that surround the cilia of the olfactory receptor cells of the olfactory epithelium) so as to facilitate absorption. Alternatively or additionally, the compound can be formulated with a carrier and/or other substances that foster dissolution of the agent within nasal secretions, including without limitation fatty acids (e.g., palmitic acid), gangliosides (e.g., GM-1), phospholipids (e.g., phosphatidylserine, and emulsifiers (e.g., polysorbate 80).

Optionally, drug solubilizers can be included in the pharmaceutical composition to improve the solubility of the compound and/or to reduce the likelihood of disruption of nasal membranes which can be caused by application of other substances, for example, lipophilic odorants. Suitable solubilizers include but are not limited to amorphous mixtures of cyclodextrin derivatives such as hydroxypropylcylodextrins (see, for example, Pitha et al., (1988) Life Sciences 43:493-502.

In representative embodiments, the compound is lipophilic to promote absorption. Uptake of non-lipophilic compounds can be enhanced by combination with a lipophilic substance. Lipophilic substances that can enhance delivery of the compound across the nasal mucus include but are not limited to fatty acids (e.g., palmitic acid), gangliosides (e.g., GM-1), phospholipids (e.g., phosphatidylserine), and emulsifiers (e.g., polysorbate 80), bile salts such as sodium deoxycholate, and detergent-like substances including, for example, polysorbate 80 such as Tween™, octoxynol such as Triton™ X-100, and sodium tauro-24,25-dihydrofusidate (STDHF). See Lee et al., Biopharm., April 1988 issue:3037.

In particular embodiments of the invention, the active compound is combined with micelles comprised of lipophilic substances. Such micelles can modify the permeability of the nasal membrane to enhance absorption of the compound. Suitable lipophilic micelles include without limitation gangliosides (e.g., GM-1 ganglioside), and phospholipids (e.g., phosphatidylserine). Bile salts and their derivatives and detergent-like substances can also be included in the micelle formulation. The active compound can be combined with one or several types of micelles, and can further be contained within the micelles or associated with their surface.

Alternatively, the active compound can be combined with liposomes (lipid vesicles) to enhance absorption. The active compound can be contained or dissolved within the liposome and/or associated with its surface. Suitable liposomes include phospholipids (e.g., phosphatidylserine) and/or gangliosides (e.g., GM-1). For methods to make phospholipid vesicles, see for example, U.S. Pat. No. 4,921,706 to Roberts et al., and U.S. Pat. No. 4,895,452 to Yiournas et al. Bile salts and their derivatives and detergent-like substances can also be included in the liposome formulation.

In representative embodiments, the pH of the pharmaceutical composition ranges from about 2, 3, 3.5 or 5 to about 7, 8 or 10. Exemplary pH ranges include without limitation from about 2 to 8, from about 3.5 to 7, and from about 5 to 7. Those skilled in the art will appreciate that because the volume of the pharmaceutical composition administered is generally small, nasal secretions may alter the pH of the administered dose, since the range of pH in the nasal cavity can be as wide as 5 to 8. Such alterations can affect the concentration of un-ionized drug available for absorption. Accordingly, in representative embodiments, the pharmaceutical composition further comprises a buffer to maintain or regulate pH in situ. Typical buffers include but are not limited to acetate, citrate, prolamine, carbonate and phosphate buffers.

In embodiments of the invention, the pH of the pharmaceutical composition is selected so that the internal environment of the nasal cavity after administration is on the acidic to neutral side, which (1) can provide the active compound in an un-ionized form for absorption, (2) prevents growth of pathogenic bacteria in the nasal passage that is more likely to occur in an alkaline environment, and (3) reduces the likelihood of irritation of the nasal mucosa.

For liquid and powder sprays or aerosols, the pharmaceutical composition can be formulated to have any suitable and desired particle size. In illustrative embodiments, the majority and/or the mean size of the particles or droplets range in size from greater than about 1, 2.5, 5, 10 or 15 microns and/or less than about 25, 30, 40, 50, 60 or 75 microns. Representative examples of suitable ranges for the majority and/or mean particle or droplet size include, without limitation, from about 5 to 50 microns, from about 20 to 40 microns, and from about 10 to 30 microns, which facilitate the deposition of an effective amount of the active compound in the nasal cavity (e.g., on the olfactory epithelium and/or in the sinus region). In general, particles or droplets smaller than about 5 microns will be deposited in the trachea or even the lung, whereas particles or droplets that are about 50 microns or larger generally do not reach the nasal cavity and are deposited in the anterior nose.

According to particular methods of intranasal delivery, it is desirable to prolong the residence time of the pharmaceutical composition in the nasal cavity (e.g., on the olfactory epithelium and/or in the sinus region), for example, to enhance absorption. Thus, the pharmaceutical composition can optionally be formulated with a bioadhesive polymer, a gum (e.g., xanthan gum), chitosan (e.g., highly purified cationic polysaccharide), pectin (or any carbohydrate that thickens like a gel or emulsifies when applied to nasal mucosa), a microsphere (e.g., starch, albumin, dextran, cyclodextrin), gelatin, a liposome, carbamer, polyvinyl alcohol, alginate, acacia, chitosans and/or cellulose (e.g., methyl or propyl; hydroxyl or carboxy; carboxymethyl or hydroxylpropyl), which are agents that enhance residence time in the nasal cavity. As a further approach, increasing the viscosity of the dosage formulation can also provide a means of prolonging contact of the agent with nasal epithelium. The pharmaceutical composition can optimally be formulated as a nasal emulsion, ointment or gel, which offer advantages for local application because of their viscosity.

Moist and highly vascularized membranes can facilitate rapid absorption; consequently, the pharmaceutical composition can optionally comprise a humectant, particularly in the case of a gel-based composition so as to assure adequate intranasal moisture content. Examples of suitable humectants include but are not limited to glycerin or glycerol, mineral oil, vegetable oil, membrane conditioners, soothing agents, and/or sugar alcohols (e.g., xylitol, sorbitol; and/or mannitol). The concentration of the humectant in the pharmaceutical composition will vary depending upon the agent selected and the formulation.

The pharmaceutical composition can also optionally include an absorption enhancer, such as an agent that inhibits enzyme activity, reduces mucous viscosity or elasticity, decreases mucociliary clearance effects, opens tight junctions, and/or solubilizes the active compound. Chemical enhancers are known in the art and include chelating agents (e.g., EDTA), fatty acids, bile acid salts, surfactants, and/or preservatives. Enhancers for penetration can be particularly useful when formulating compounds that exhibit poor membrane permeability, lack of lipophilicity, and/or are degraded by aminopeptidases. The concentration of the absorption enhancer in the pharmaceutical composition will vary depending upon the agent selected and the formulation.

The pharmaceutical composition can optionally contain an odorant (e.g., as described in EP 0 504 263 B1) to provide a sensation of odor, to aid in inhalation of the composition so as to promote delivery to the olfactory epithelium and/or to trigger transport by the olfactory neurons.

The pharmaceutical composition can be delivered in any suitable volume of administration. In representative embodiments of the invention, the administration volume for intranasal delivery ranges from about 25 microliters to as much as 3 ml or more, preferably 25 microliters to 200 microliters, and more preferably from about 50 to 150 microliters. Typically, the administration volume is selected to be small enough to allow for the dissolution of an effective amount of the active compound but sufficiently large to prevent therapeutically significant amounts of compounds from escaping from the anterior chamber of the nose and/or draining into the throat, post nasally.

Any suitable method of intranasal delivery can be employed for delivery of the compound. To illustrate, the pharmaceutical composition can be administered intranasally as (1) nose drops, (2) powder or liquid sprays or aerosols, (3) liquids or semisolids by syringe, (4) liquids or semisolids by swab, pledget or other similar means of application, (5) a gel, cream or ointment, (6) an infusion, or (7) by injection, or by any means now known or later developed in the art. In particular embodiments, the method of delivery is by spray or aerosol.

In representative embodiments, the pharmaceutical formulation is directed upward during administration, to enhance delivery to the upper third (e.g., the olfactory epithelium) and the side walls (e.g., nasal epithelium) of the nasal cavity.

The methods of intranasal delivery can be carried out once or multiple times, and can further be carried out daily, every other day, etc., with a single administration or multiple administrations per day of administration, (e.g., 2, 3, 4 or more times per day of administration). In other embodiments, the methods of the invention can be carried out on an as-needed by self-medication.

Administration of the compound may also take place using a nasal tampon or nasal sponge.

The invention also encompasses pharmaceutical compositions formulated for pulmonary administration comprising one or more compounds that increases tricarboxylic acid (TCA) cycle flux through acetyl-CoA in the hypothalamus of a mammal in a pharmaceutically acceptable carrier. Compounds that can be used in the pharmaceutical compositions of the invention are discussed in the preceding section. The pharmaceutical composition can modulate the amount and/or activity of an acetyl-CoA-decreasing molecule and/or a LC-CoA-increasing molecule (each as described above). The one or more compounds can individually be prodrugs that are converted to the active compound in vivo. In particular embodiments, the invention provides a pharmaceutical composition formulated for pulmonary administration comprising a compound that increases the activity of an acetyl-CoA-increasing molecule or decreases the activity of an acetyl-CoA-decreasing molecule in the hypothalamus of the mammal. Such compounds that elevate intracellular LC-CoA levels are known in the art and are discussed in more detail hereinabove.

“Pulmonary administration” or “administration to the lungs,” and like terms, are used interchangeably herein. These terms refer to delivery of a composition to the lung(s) of a subject, e.g., the bronchi, bronchioli and/or alveoli. Generally, pharmaceutical compositions administered to the respiratory tract by oral or nasal inhalation travel through the upper airways (oropharynx and larynx), the lower airways which include the trachea followed by bifurcations into the bronchi and bronchioli and through the terminal bronchioli which in turn divide into respiratory bronchioli leading then to the ultimate respiratory zone, the alveoli or the deep lung. Absorption through the alveoli results from rapid dissolution of the formulation in the ultra-thin (0.1 μm) fluid layer of the alveolar lining of the lung. According to certain aspects of the invention, pulmonary administration is to the deep lung or alveoli. In particular embodiments of the invention, at least about 5%, 10%, 20%, 30%, 40%, 50% or more of the mass of particles deposits in the deep lung or alveoli. Deposition in the deep lung, for example the alveoli, can be influenced by a variety of factors including the delivery method, the characteristics of the delivery device (e.g., the size of the particles produced, the delivery velocity, and the like), and the characteristics of the delivered composition. Compositions and methods for achieving enhanced delivery to the deep lung or alveoli are discussed below.

In particular embodiments, the pharmaceutical composition is administered to the subject in an effective amount, optionally, a therapeutically effective amount (each as described herein). Dosages of pharmaceutically active compositions can be determined by methods known in the art, see, e.g., Remington's Pharmaceutical Sciences (Maack Publishing Co., Easton, Pa; 18^{th} edition, 1990).

A therapeutically effective amount will vary with the age and general condition of the subject, the efficiency of the delivery method (i.e., the percent of the dose that is deposited in the target area), the severity of the condition being treated, the particular compound or composition being administered, the duration of the treatment, the nature of any concurrent treatment, the carrier used, and like factors within the knowledge and expertise of those skilled in the art. As appropriate, a therapeutically effective amount in any individual case can be determined by one of ordinary skill in the art by reference to the pertinent texts and literature and/or by using routine experimentation (see, e.g., Remington, The Science and Practice of Pharmacy (20^{th} ed. 2000)).

As a general proposition, a dosage from about 0.01 to about 0.1, 1, 5, 10, 20, 50, 75 or 100 mg active agent/kg body weight will have therapeutic efficacy, with all weights being calculated based upon the weight of the active ingredient, including salts.

Aerosol dosage, formulations and delivery systems may be selected as described, for example, in Gonda, “Aerosols for delivery of therapeutic and diagnostic agents to the respiratory tract,” in Critical Reviews in Therapeutic Drug Carrier Systems, 6: 273-313, 1990; and in Moren, “Aerosol dosage forms and formulations,” in: Aerosols in Medicine. Principles, Diagnosis and Therapy, Moren, et al., Eds, Esevier, Amsterdam, 1985.

The pharmaceutical composition can be delivered in any suitable volume or mass (weight) of administration. In representative embodiments of the invention, the administration volume of liquid particles (e.g., liquid aerosol particles) in a single administration suitable for pulmonary delivery ranges from several microliters to several milliliters (e.g., from about 3 microliters to about 3, 4 or 5 milliliters). In other particular embodiments, the mass of solid particles (e.g., solid aerosol particles) in a single administration suitable for pulmonary delivery ranges from several micrograms to several milligrams (e.g., about 3 micrograms to about 3, 4 or 5 milligrams).

Pulmonary administration of any suitable formulation known or later discovered in the art is contemplated. For example, the composition can be formulated for nasal or oral administration to the lungs. In certain embodiments, the composition is formulated for oral inhalation. In other exemplary embodiments, the composition is administered as an aerosol solution, a suspension, or a dry powder of respirable particles containing the active agent, which the subject inhales. The respirable particles can be liquid or solid; optionally, the respirable particles are a dry powder or liquid aerosol. Further, in certain embodiments the respirable particles described above can be administered by oral inhalation.

For the purposes of the present invention, the term “aerosol” includes any gas-borne suspended phase, which is capable of being inhaled into the bronchioles or nasal passages. Specifically, an aerosol includes a gas-borne suspension of droplets, as can be produced in a metered dose inhaler, nebulizer, mist sprayer, or the like. The term “aerosol” also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn, et al. (1992) J. Pharmacol. Toxicol. Methods 27:143-159.

In particular embodiments, the pulmonary formulation comprises a dispersible dry powder. “Dispersibility” or “dispersible” or equivalent terms means a dry powder having a moisture content of less than about 10% by weight (% w) water, usually below about 5% w, or less than about 3% w; a particle size of about 1-5 μm mass median diameter (MMD), 1-4 μm MMD or 1-3 μm MMD; a delivered dose of about >5%, >10%, >15%, >20%, >30%, >40% or >50%; and an aerosol particle size distribution of about 1-5 μm mass median aerodynamic diameter (MMAD), 1.5-4.5 μm MMAD, or 1.5-3 μm MMAD. Methods and compositions for improving dispersibility are disclosed in U.S. Pat. Nos. 6,136,346; 6,358,530 and 6,582,729; the disclosures of which are hereby incorporated by reference.

The term “powder” as used herein means a composition that consists of finely dispersed solid particles that are free flowing and capable of being readily dispersed in an inhalation device and subsequently inhaled by a subject so that the particles reach the lungs, and optionally permit penetration into the alveoli. In particular embodiments, the average particle size is less than about 10, 7.5, or 5 μm in diameter with a relatively uniform spheroidal shape distribution. Generally, the particle size distribution is between about 0.1 μm and about 5 μm, particularly about 0.3 μm to about 5 μm or about 1 μm to about 3 μm.

The term “dry” means that the composition has a moisture content such that the particles are readily dispersible in an inhalation device to form an aerosol. This moisture content is generally below about 10% by weight (% w) water, usually below about 5% w or below about 3% w.

In the dry state, the powder may be in crystalline or amorphous form.

A therapeutically effective amount of active pharmaceutical will vary in the composition depending on the biological activity of the compound(s) employed and the amount needed in a unit dosage form. Because the composition is dispersible, it is generally advantageous that it is manufactured in a unit dosage form in a manner that allows for ready manipulation by the formulator and by the consumer. A unit dosage will typically be between about 0.5 mg and 15 mg, more particularly between about 2 mg and 10 mg, of total material in the dry powder composition.

Aerosols of liquid particles containing the active agent can be produced by any suitable aerosolization means, such as with a pressure-driven aerosol nebulizer, an electrostatic nebulizer, an ultrasonic nebulizer, a pressured/volatile gas-filled metered dose inhaler (MDI), a piston-driven system with a grid or laser-drilled holes, or devices that rely upon the subject's inspiratory flow, as are known to those of skill in the art. See, e.g., U.S. Pat. No. 4,501,729.

Aerosols of solid particles containing the active agent can likewise be produced with any solid particulate medicament aerosol generator, by techniques known in the pharmaceutical art. See, for example, U.S. Pat. No. 6,169,068, U.S. Pat. No. 6,334,999 and U.S. Pat. No. 6,797,258; the disclosure of which is incorporated herein by reference.

As further examples, solid particles can be delivered from an inhalation device such as a dry powder inhaler (DPI) or a MDI.

Other suitable inhalers are described in U.S. Pat. No. 4,069,819, U.S. Pat. No. 4,995,385, and U.S. Pat. No. 5,997,848. Further examples include, but are not limited to, the Spinhaler®. (Fisons, Loughborough, U.K.), Rotahaler®. (Glaxo-Wellcome, Research Triangle Technology Park, N.C.), FlowCaps®. (Hovione, Loures, Portugal), Inhalator® (Boehringer-Ingelheim, Germany), and the Aerolizer® (Novartis, Switzerland), the diskhaler (Glaxo-Wellcome, RTP, N.C.) and others, such as known to those skilled in the art.

Dry powder dispersion devices for medicaments are described in a number of patent documents. U.S. Pat. No. 3,921,637 describes a manual pump with needles for piercing through a single capsule of powdered medicine. The use of multiple receptacle disks or strips of medication is described in European Patent Application No. EP 0 467 172 (where a reciprocatable punch is used to open a blister pack); International Patent Publication Nos. WO 91/02558 and WO 93/09832; U.S. Pat. Nos. 4,627,432; 4,811,731; 5,035,237; 5,048,514; 4,446,862; 5,048,514; and 4,446,862. Other patents that show puncturing of single medication capsules include U.S. Pat. Nos. 4,338,931; 3,991,761; 4,249,526; 4,069,819; 4,995,385; 4,889,114; and 4,884,565; and European Patent Application No. EP 469 814. International Patent Publication No. WO 90/07351 describes a hand-held pump device with a loose powder reservoir.

A dry powder sonic velocity disperser is described in Witham and Gates, Dry Dispersion with Sonic Velocity Nozzles, presented at the workshop on Dissemination Techniques for Smoke and Obscurants, Chemical Systems Laboratory, Aberdeen Proving Ground, Maryland, Mar. 14-16, 1983.

U.S. Pat. Nos. 4,926,852 and 4,790,305 describe a type of “spacer” for use with a metered dose inhaler. The spacer defines a large cylindrical volume which receives an axially directed burst of drug from a propellant-driven drug supply. U.S. Pat. No. 5,027,806 is an improvement over the '852 and '305 patents, having a conical holding chamber that receives an axial burst of drug. U.S. Pat. No. 4,624,251 describes a nebulizer connected to a mixing chamber to permit a continuous recycling of gas through the nebulizer. European Patent Application No. 0 347 779 describes an expandable spacer for a metered dose inhaler having a one-way valve on the mouthpiece. International Patent Publication No. WO 90/07351 describes a dry powder oral inhaler having a pressurized gas source (a piston pump) which draws a measured amount of powder into a venturi arrangement.

Stribling et al. (1992) J. Biopharm. Sci. 3:255-263, describes the aerosol delivery of plasmids carrying a chloramphenicol acetyltransferase (CAT) reporter gene to mice. The plasmids were incorporated in DOTMA (N-[1-(2-, 3-dioleyloxy)propyl]-N,N,N-trimethylammonium chloride) or cholesterol liposomes, and aqueous suspensions of the liposomes were nebulized into a small animal aerosol delivery chamber. Mice breathing the aerosol were found to at least transiently express CAT activity in their lung cells. Rosenfeld et al. (1991) Science: 252:431-434, describes the in vivo delivery of an alpha-1 antitrypsin gene to rats, with secretion of the gene product being observable for at least one week. The gene was diluted in saline and instilled directly into the rat trachea.

Patton and Platz (1992) Adv. Drug Deliver. Rev. 8:179-196, describe methods for delivering proteins and polypeptides by inhalation to the deep lung.

The aerosolization of protein therapeutic agents, including alpha-1 antitrypsin, is disclosed in European Patent Application No. EP 0 289 336. In general, nebulizers can be advantageous for delivery of liquid compositions comprising polypeptides, as nebulizers are gentler on the pharmaceutical composition than, for example, a MDI device.

There are several considerations that bear upon the design and operation of the delivery device. To illustrate, unlike dry powder administration, for liquids it is the device that determines particle size, usually as a function of the diameter of the delivery port, mesh or grid. Liquid particles (i.e., droplets) having a desired size as described herein can be achieved by selection of a suitable delivery device based on considerations well-known in the art.

Delivery velocity is another factor to consider for pulmonary delivery. Even particles of an optimum size will rebound from the soft palate, and therefore not travel down the trachea, if they are delivered at too high a velocity. On the other hand, particles that are too slowly will not enter the respiratory tract at all (e.g., in the case of oral inhalation, they will land on the tongue).

Delivery devices and methods can also be selected to time delivery of the pharmaceutical composition with the breathing cycle. Some devices incorporate firmware that measures the timing of the patient's breathing cycle and optimizes pulsed drug delivery to achieve efficient delivery. More commonly, devices (included pulsed nebulizers, MDIs and dry powder devices) rely on a learned coordination by the patient of drug delivery with inspiration. MDIs are available that provide a mixing cylinder placed between the delivery device and the mouthpiece to improve dispersion of the aerosol and reduce the need to time delivery with the breathing cycle.

As yet another approach, some devices incorporate a heating device to warm the pharmaceutical composition to body temperature during delivery, which results in more efficient pulmonary delivery.

The compositions of the invention can be formulated for pulmonary administration in a pharmaceutical carrier in accordance with known techniques. See, e.g., Remington, The Science And Practice of Pharmacy (20^{th} edition, 2000). Suitable nontoxic pharmaceutically acceptable carriers for pulmonary administration will be apparent to those skilled in the art of pulmonary pharmaceutical formulations (see, e.g., Remington's Pharmaceutical Sciences (Maack Publishing Co., Easton latest edition).

The composition may optionally be combined with pharmaceutical carriers or excipients that are suitable for pulmonary administration. Such carriers may serve simply as bulking agents when it is desired to reduce the pharmaceutical concentration in the powder which is being delivered to a patient, but may also serve to enhance the stability of the compositions and to improve the dispersibility of the powder within a powder dispersion device in order to provide more efficient and reproducible delivery of the powder and to improve handling characteristics such as flowability and consistency to facilitate manufacturing and powder filling.

Such carrier materials may be combined with the drug prior to spray drying, e.g., by adding the carrier material to the purified bulk solution. In that way, the carrier particles will be formed simultaneously with the drug particles to produce a homogeneous powder. Alternatively, the carriers may be separately prepared in a dry powder form and combined with the dry powder drug by blending. The powder carriers will usually be crystalline (to avoid water absorption), but might in some cases be amorphous or mixtures of crystalline and amorphous. The size of the carrier particles may be selected to improve the flowability of the drug powder, typically being in the range from about 25 μm to 100 μm. One suitable carrier material is crystalline lactose having a size in the above-stated range.

The active compound(s) can be present in the formulation in any suitable amount, for example, a range from about 0.05, 0.1, 0.5 or 1% to about 50, 60, 70, 80, 90, 95, 97 or 99% (w/w or w/v).

The compound can have any suitable molecular weight. According to certain embodiments of the invention, the compound has a molecular weight of less than about 10 kiloDalton (kD), 7.5 kD, 5 kD, 2 kD, 1 kD, 500 Daltons or less.

The pharmaceutical composition can further have any suitable osmolarity, for example, in the range of about 100 to 600 mOsM, about 150 to 450 mOsM, or about 175 to 310 mOsM.

The formulation can further comprise one or more component(s) that promote(s) the fast release of the active compound(s) into the blood stream. In one embodiment, a therapeutic plasma concentration is achieved in less than about 10 minutes, 5 minutes, 2 minutes or even sooner after administration.

In particular embodiments, the formulation includes one or more phospholipids, such as, for example, a phosphatidylcholine, phosphatidylethanolamine, phosphatidylglycerol, phosphatidylserine, phosphatidylinositol or a combination thereof. For example, the phospholipids can be endogenous to the lung. Combinations of phospholipids can also be employed. Specific examples of phospholipids are shown in Table 1.

TABLE 1

| | |
|---|---|
| Dilaurylolyphosphatidylcholine (C12; 0) | DLPC |
| Dimyristoylphosphatidylcholine (C14; 0) | DMPC |
| Dipalmitoylphosphatidylcholine (C16:0) | DPPC |
| Distearoylphosphatidylcholine (18:0) | DSPC |
| Dioleoylphosphatidylcholine (C18:1) | DOPC |
| Dilaurylolylphosphatidylglycerol | DLPG |
| Dimyristoylphosphatidylglycerol | DMPG |
| Dipalmitoylphosphatidylglycerol | DPPG |
| Distearoylphosphatidylglycerol | DSPG |
| Dioleoylphosphatidylglycerol | DOPG |
| Dimyristoyl phosphatidic acid | DMPA |
| Dipalmitoyl phosphatidic acid | DPPA |
| Dimyristoyl phosphatidylethanolamine | DMPE |
| Dipalmitoyl phosphatidylethanolamine | DPPE |
| Dimyristoyl phosphatidylserine | DMPS |
| Dipalmitoyl phosphatidylserine | DPPS |
| Dipalmitoyl sphingomyelin | DPSP |
| Distearoyl sphingomyelin | DSSP |

The phospholipid can be present in the formulation in any suitable amount, e.g., an amount ranging from about 0, 1, 5 or 10% to about 50, 60, 70, 80 or 90% (w/w or w/v).

The phospholipids or combinations thereof can be selected to impart rapid or controlled-release properties to the formulation. Particles having controlled-release properties and methods of modulating release of a biologically active agent are described in U.S. patent application Ser. No. 09/644,736 and U.S. patent Publication No. 20010036481; the disclosures of which are incorporated herein by reference. Rapid release can be obtained, for example, by including in the formulation phospholipids characterized by low transition temperatures.

Rapid release can also be achieved by administering formulations comprising nanoparticles (e.g., because of large surface area) and formulations in the form of solutions.

Nanoparticles, microspheres, cyclodextrins and liposomes can be used as vehicles for controlled-release delivery.

In another embodiment, rapid and controlled-release of the active compound(s) are coupled in a single course of therapy.

The formulation can further include a surfactant. As used herein, the term “surfactant” refers to any agent that preferentially absorbs to an interface between two immiscible phases, such as the interface between water and an organic polymer solution, a water/air interface or organic solvent/air interface. Surfactants generally possess a hydrophilic moiety and a lipophilic moiety, such that, upon absorbing to microparticles, they tend to present moieties to the external environment that do not attract similarly-coated particles, thus reducing particle agglomeration. Surfactants may also promote absorption of a therapeutic or diagnostic agent and increase bioavailability of the agent.

In addition to lung surfactants, such as, for example, the phospholipids discussed above, suitable surfactants include but are not limited to hexadecanol; fatty alcohols such as polyethylene glycol (PEG) and acetyl alcohol; polyoxyethylene-9-lauryl ether; a surface active fatty acid, such as palmitic acid or oleic acid; glycocholate; surfactin; a poloxomer; a sorbitan fatty acid ester such as sorbitan trioleate (Span 85); and tyloxapol.

The surfactant can be present in the formulation in any suitable amount, for example, an amount ranging from about 0, 1, 5 or 10% to about 50, 60, 70, 80 or 90% (w/w or w/v).

Methods of preparing and administering particles including surfactants, in particular phospholipids, are disclosed in U.S. Pat. No 5,855,913 and U.S. Pat. No. 5,985,309; the disclosures of both are incorporated herein by reference in their entireties.

Additives can be included for conformational stability during spray drying and for improving dispersibility of powders. One such group of additives includes amino acid(s), in particular, hydrophobic amino acid(s). Suitable amino acids include naturally occurring and non-naturally occurring amino acids. Specific examples of amino acids which can be employed include, but are not limited to: alanine, glycine, proline, cysteine, methionine, valine, leucine, tyrosine, isoleucine, phenylalanine, and tryptophan. Non-naturally occurring amino acids include, for example, β-amino acids. Both D, L and racemic configurations of amino acids can be employed. Suitable amino acids can also include amino acid analogs. As used herein, an amino acid analog includes the D or L configuration of an amino acid having the following formula: —NH—CHR—CO— wherein R is an aliphatic group, a substituted aliphatic group, a benzyl group, a substituted benzyl group, an aromatic group or a substituted aromatic group and wherein R does not correspond to the side chain of a naturally-occurring amino acid. As used herein, aliphatic groups include straight chained, branched or cyclic C1-C8 hydrocarbons which are completely saturated, which contain one or two heteroatoms such as nitrogen, oxygen or sulfur and/or which contain one or more units of unsaturation. Aromatic groups include carbocyclic aromatic groups such as phenyl and naphthyl and heterocyclic aromatic groups such as imidazolyl, indolyl, thienyl, furanyl, pyridyl, pyranyl, oxazolyl, benzothienyl, benzofuranyl, quinolinyl, isoquinolinyl and acridintyl.

Suitable substituents on an aliphatic, aromatic or benzyl group include —OH, halogen (—Br, —Cl, —I and —F) —O (aliphatic, substituted aliphatic, benzyl, substituted benzyl, aryl or substituted aryl group), —CN, —NO₂, —COOH, —NH₂, —NH(aliphatic group, substituted aliphatic, benzyl, substituted benzyl, aryl or substituted aryl group), —N(aliphatic group, substituted aliphatic, benzyl, substituted benzyl, aryl or substituted aryl group)₂, —COO(aliphatic group, substituted aliphatic, benzyl, substituted benzyl, aryl or substituted aryl group), —CONH₂, —CONH(aliphatic, substituted aliphatic group, benzyl, substituted benzyl, aryl or substituted aryl group), —SH, —S(aliphatic, substituted aliphatic, benzyl, substituted benzyl, aromatic or substituted aromatic group) and —NH—C(═NH)—NH₂. A substituted benzylic or aromatic group can also have an aliphatic or substituted aliphatic group as a substituent. A substituted aliphatic group can also have a benzyl, substituted benzyl, aryl or substituted aryl group as a substituent. A substituted aliphatic, substituted aromatic or substituted benzyl group can have one or more substituents. Modifying an amino acid substituent can increase, for example, the lipophilicity or hydrophobicity of natural amino acids that are hydrophilic.

A number of suitable amino acids, amino acid analogs and salts thereof can be obtained commercially. Others can be synthesized by methods known in the art. Synthetic techniques are described, for example, in Green and Wuts, “Protecting Groups in Organic Synthesis”, John Wiley and Sons, Chapters 5 and 7, 1991.

Hydrophobicity is generally defined with respect to the partition of an amino acid between a nonpolar solvent and water. Hydrophobic amino acids are those amino acids that show a preference for the nonpolar solvent. Relative hydrophobicity of amino acids can be expressed on a hydrophobicity scale on which glycine has the value 0.5. On such a scale, amino acids that have a preference for water have values below 0.5 and those that have a preference for nonpolar solvents have a value above 0.5. As used herein, the term “hydrophobic amino acid” refers to an amino acid that on the hydrophobicity scale has a value greater or equal to 0.5, in other words, has a tendency to partition in the nonpolar solvent that is at least equal to that of glycine.

In representative embodiments, combinations of hydrophobic amino acids are employed. Furthermore, in other embodiments, combinations of hydrophobic and hydrophilic (preferentially partitioning in water) amino acids, where the overall combination is hydrophobic, are employed.

The amino acid can be present in the pulmonary formulations in any suitable amount, for example, an amount of at least about 10% (w/w or w/v). In particular embodiments, the amino acid is present in the formulation in an amount ranging from about 20% to about 80% (w/w or w/v). The salt of a hydrophobic amino acid can be present in the formulation in any suitable amount, for example, an amount of at least about 10% (w/w or w/v). In illustrative embodiments, the amino acid salt is present in the formulation in an amount ranging from about 20% to about 80% (w/w or w/v). Methods of forming and delivering particles that include an amino acid are described in U.S. Pat. No. 6,586,008 and U.S. patent application Ser. No. 09/644,320; the teachings of which are incorporated herein by reference.

In another embodiment of the invention, the formulation includes a carboxylate moiety and/or a multivalent metal salt. Such compositions are described, for example, in U.S. Pat. No. 6,749,835; the disclosure of which is incorporated herein by reference. In one particular embodiment, the formulation includes sodium citrate and/or calcium chloride.

The pulmonary formulation can further comprise carriers including but not limited to stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-β-cyclodextrin; and polysaccharides, such as raffinose, maltodextrins, dextrans, and the like; alditols, such as mannitol, xylitol, and the like. Suitable polypeptides include aspartame. Amino acids include alanine and glycine.

The pharmaceutical composition can have any suitable pH. In representative embodiments, the pH of the pharmaceutical composition ranges from about 4, 4.5, 5 or 5.5 to about 6, 6.5, 7, 7.5 or 8. Exemplary pH ranges include without limitation from about pH 4.5 to about pH 8 and from about pH 5.5 to about pH 7.5. Those skilled in the art will appreciate that because the volume of the pharmaceutical composition administered is generally small (e.g., less than 5 milliliters), secretions from the respiratory tract may alter the pH of the administered dose. Such alterations can affect the concentration of un-ionized drug available for absorption. Accordingly, in representative embodiments, the pharmaceutical composition further comprises a buffer to maintain or regulate pH in situ. Typical buffers include but are not limited to organic salts, e.g., prepared from organic acids and bases, such as acetate, citrate, prolamine, carbonate, ascorbate and phosphate buffers.

Other materials, including materials that promote fast release kinetics of the active compound can also be employed. For example, biocompatible, and optionally biodegradable polymers can be employed. Illustrative formulations including such polymeric materials are described in U.S. Pat. No. 5,874,064; the disclosure of which is incorporated herein by reference in its entirety.

The formulation can further include a material such as, for example, dextran, polysaccharides, lactose, trehalose, cyclodextrins, proteins, peptides, polypeptides, fatty acids, inorganic compounds and/or phosphates.

To extend shelf life, preservatives can optionally be added to the pharmaceutical composition. Suitable preservatives include but are not limited to benzyl alcohol, parabens, thimerosal, chlorobutanol and benzalkonium chloride, and combinations of the foregoing. The concentration of the preservative will vary depending upon the preservative used, the compound being formulated, the formulation, and the like. In representative embodiments, the preservative is present in an amount of 2% by weight or less. In certain embodiments, the pharmaceutical composition is sufficiently stable as to not require the addition of preservatives. The absence of preservatives can be advantageous since preservatives may raise safety and toxicity issues, especially to the lung.

In particular embodiments, the formulation is substantially free of any penetration enhancers. The use of penetration enhancers in formulations for the lungs is often undesirable because the epithelial cell layer in the lung can be adversely affected by such surface active compounds.

As another option, the composition can comprise a flavoring agent, e.g., to enhance the taste and/or acceptability of the composition to the subject.

In the case of active compounds comprising polypeptides, it is generally desirable that the compound be shielded from leukocyte proteases in the lung and/or have a structure that is resistant to proteolytic degradation. To illustrate, the compound can be protected from proteolytic cleavage by encapsulation, for example, in lysosomes. As another option, the compound can be formulated with a protease inhibitor, such as benzamidine or a derivative thereof (see, e.g., Pauls et al., (2004) Front. Med. Chem. 1:129-152). As still another approach, polypeptides can be synthesized with modified peptide bonds and/or with blocked or otherwise modified amino and/or carboxyl termini that are resistant to proteolytic cleavage.

In representative embodiments, the active compound is lipophilic to promote absorption. In general, nonpolar compounds more readily cross the mucosal lining and the epithelial cell layer in the lungs. In other embodiments, uptake of non-lipophilic compounds is enhanced by combination with a lipophilic substance. Lipophilic substances that can enhance absorption of the compound include but are not limited to fatty acids (e.g., palmitic acid), gangliosides (e.g., GM-1), phospholipids (e.g., phosphatidylserine), and emulsifiers (e.g., polysorbate 80), bile salts such as sodium deoxycholate, and detergent-like substances including, for example, polysorbate 80 such as Tween™, octoxynol such as Triton™ X-100, and sodium tauro-24,25-dihydrofusidate (STDHF). See Lee et al., Biopharm., April 1988 issue: 3037.

In particular embodiments of the invention, the active compound is combined with micelles comprised of lipophilic substances, e.g., to achieve a uniform emulsion. Such micelles can modify the permeability of the alveoli membrane to enhance absorption of the compound. Suitable lipophilic micelles include without limitation gangliosides (e.g., GM-1 ganglioside), and phospholipids (e.g., phosphatidylserine). Bile salts and their derivatives and detergent-like substances can also be included in the micelle formulation. The active compound can be combined with one or several types of micelles, and can further be contained within the micelles or associated with their surface.

Alternatively, the active compound can be combined with liposomes (lipid vesicles) to enhance absorption. The active compound can be contained or dissolved within the liposome and/or associated with its surface. Suitable liposomes include phospholipids (e.g., phosphatidylserine) and/or gangliosides (e.g., GM-1). For methods of making phospholipid vesicles, see for example, U.S. Pat. No. 4,921,706 to Roberts et al., and U.S. Pat. No. 4,895,452 to Yiournas et al. Bile salts and their derivatives and detergent-like substances can also be included in the liposome formulation.

The pharmaceutical composition can be selected to enhance delivery to the desired target regions, e.g., the deep lung or alveoli. In particular embodiments, the liquid or dry powder particles, optionally liquid or dry powder aerosol particles, have a tap density less than about 0.4, 0.2 or even 0.1 g/cm³. Particles that have a tap density of less than about 0.4 g/cm³ are referred to herein as “aerodynamically light particles”. Tap density can be measured by using instruments known to those skilled in the art such as but not limited to the Dual Platform Microprocessor Controlled Tap Density Tester (Vankel, N.C.) or a GeoPyc™ instrument (Micrometrics Instrument Corp., Norcross, Ga.). Tap density is a standard measure of the envelope mass density. Tap density can be determined using the method of USP Bulk Density and Tapped Density, United States Pharmacopeia convention, Rockville, Md., 10^{th} Supplement, 4950-4951, 1999. Features that can contribute to low tap density include irregular surface texture and porous structure.

The envelope mass density of an isotropic particle is defined as the mass of the particle divided by the minimum sphere envelope volume within which it can be enclosed. In one embodiment of the invention, the particles have an envelope mass density of less than about 0.4 g/cm³.

In particular embodiments, aerodynamically light particles have a size, e.g., a volume median geometric diameter (VMGD), of at least about 5 μm. In one embodiment, the VMGD is from about 5 μm to about 30 μm. In another embodiment of the invention, the particles have a VMGD ranging from about 10 μm to about 30 μm. In other embodiments, the particles have a median diameter, mass median diameter (MMD), a mass median envelope diameter (MMED) or a mass median geometric diameter (MMGD) of at least 5 μm, for example from about 5 μm to about 30 μm.

The diameter of spray-dried particles, for example, the VMGD, can be measured using an electrical zone sensing instrument such as a Multisizer IIe, (Coulter Electronic, Luton, Beds, England), or a laser diffraction instrument (for example Helos, manufactured by Sympatec, Princeton, N.J.). Other instruments for measuring particle diameter are well known in the art. The diameter of particles in a sample will range depending upon factors such as particle composition and methods of synthesis. The distribution of size of particles in a sample can be selected to permit optimal deposition to targeted sites within the lungs.

In representative embodiments, aerodynamically light liquid or dry powder particles have a “mass median aerodynamic diameter” (MMAD), also referred to herein as “aerodynamic diameter”, between about 1 μm and about 5 μm. In another embodiment of the invention, the MMAD is between about 1 μm and about 3 μm. In a further embodiment, the MMAD is between about 3 μm and about 5 μm.

Experimentally, aerodynamic diameter can be determined by employing a gravitational settling method, whereby the time for an ensemble of particles to settle a certain distance is used to infer directly the aerodynamic diameter of the particles. An indirect method for measuring the mass median aerodynamic diameter (MMAD) is the multi-stage liquid impinger (MSLI). The aerodynamic diameter, dₐₑᵣ, can be calculated from the equation:

dₐₑᵣ=d_{g θ}ρₜₐₚ

where d_{g} is the geometric diameter, for example the MMGD, and ρ is the powder density.

Particles that have a tap density less than about 0.4 g/cm³, a median geometric diameter of at least about 5 μm, and/or an MMAD of between about 1 μm and about 3 or 5 μm, are more likely of escaping inertial and gravitational deposition in the oropharyngeal region, and are targeted to the airways, particularly the deep lung. In particular embodiments, the use of larger, more porous particles can be advantageous since they are generally able to aerosolize more efficiently than smaller, denser aerosol particles. In certain embodiments of the invention, such larger more porous particles are used as a vehicle for the delivery of dry powders.

In other representative embodiments, larger particles which are less porous, but which are effectively microspheres containing a suspension of dry particles or droplets, may also possess a density less than about 0.4 g/cm³, or preferably less than about 0.15 g/cm³. Such particles are most efficiently delivered to the deep lung if they possess a geometric diameter from about 4 μm to greater than about 8 μm.

In embodiments of the invention, the particles have an MMAD of about 1 to 5 μm, more particularly about 1 to 3 μm. The particles can be liquid or dry powder.

In embodiments in which the particles to be delivered are composed of an aerosol generated from a liquid (e.g., from an aqueous solution), the particles generally have a density greater than 1 g/cm³ and less than about 1.2 g/cm³. In these embodiments, the particles typically have a geometric diameter ranging from about 1 μm to about 5 μm or from about 1 μm to about 3 μm for delivery to the deep lung or alveoli.

In other embodiments, the particles generally have a median diameter of at least about 5 μm, and more particularly about 15-20 μm, and are generally more likely to avoid phagocytic engulfment by alveolar macrophages and clearance from the lungs, due to size exclusion of the particles from the phagocytes cytosolic space. According to this embodiment, the particles generally have a low density, e.g., dry powder particles or a suspension of microspheres.

The particles can be fabricated with the appropriate material, surface roughness, diameter and tap density for localized delivery to selected regions of the lungs such as the deep lung or upper or central airways. For example, higher density or larger particles may be used for upper airway delivery, or a mixture of varying sized particles in a sample, provided with the same or different therapeutic agent may be administered to target different regions of the lung in one administration. Particles having an MMAD ranging from about 3 to about 5 μm are generally suitable for delivery to the central and upper airways. Particles having an MMAD ranging from about 1 to about 3 μm or about 5 μm are generally suitable for delivery to the deep lung. Inertial impaction and gravitational settling of aerosols are predominant deposition mechanisms in the airways and acini of the lungs during normal breathing conditions. Edwards, D. A., J Aerosol Sci., 26: 293-317 (1995). The importance of both deposition mechanisms increases in proportion to the mass of aerosols and not to particle (or envelope) volume. Since the site of aerosol deposition in the lungs is influenced by the mass of the aerosol (at least for particles of mean aerodynamic diameter greater than approximately 1 μm), diminishing the tap density by increasing particle surface irregularities and particle porosity permits the delivery of larger particle envelope volumes into the lungs, all other physical parameters being equal.

The low tap density particles have a small aerodynamic diameter in comparison to the actual envelope sphere diameter. The aerodynamic diameter, dₐₑᵣ is related to the envelope sphere diameter, d (Gonda, I., “Physico-chemical principles in aerosol delivery,” in Topics in Pharmaceutical Sciences 1991 (eds. D. J: A. Crommelin and K. K. Midha), pp. 95-117, Stuttgart: Medpharm Scientific Publishers, 1992)), by the formula:

Bₐₑᵣ≈dπp.

where the envelope mass ρ is in units of g/cm³. Maximal deposition of monodispersed aerosol particles in the alveolar region of the human lung (˜60%) occurs for an aerodynamic diameter of approximately dₐₑᵣ=3 μm. Heyder, J. et al., J Aerosol Sci., 17: 811-825 (1986). Due to their small envelope mass density, the actual diameter d of aerodynamically light particles comprising a monodisperse inhaled powder that will exhibit maximum deep-lung deposition is:

d=3/op μm (where ρ<1 g/cm³);

where d is greater than 3 μm. For example, aerodynamically light particles that display an envelope mass density, ρ=0.1 g/cm³, will exhibit a maximum deposition for particles having envelope diameters as large as 9.5 μm. The increased particle size diminishes interparticle adhesion forces. Visser, J., Powder Technology, 58: 1-10. Thus, large particle size generally increases efficiency of aerosolization to the deep lung for particles of low envelope mass density, in addition to contributing to lower phagocytic losses.

The aerodynamic diameter can be calculated to provide for maximum deposition within the lungs. Previously this was achieved by the use of very small particles of less than about five microns in diameter, preferably between about one and about three microns, which particles may then be subject to phagocytosis. For the delivery of formulations composed of microspheres or particles formulated for the delivery of a dry powder, particles that have a larger diameter, but that are sufficiently light (hence the characterization “aerodynamically light”), can result in an equivalent delivery to .the lungs, with a lower susceptibility to phagocystosis. Improved delivery can be obtained by using particles with a rough or uneven surface, which also have a lower susceptibility for phagocystosis. In another embodiment of the invention, the particles have an envelope mass density, also referred to herein as “mass density” of less than about 0.4 g/cm³. In particular embodiments, particles have a mean diameter of between about 5 μm and about 30 μm. Mass density and the relationship between mass density, mean diameter and aerodynamic diameter are discussed in U.S. application Ser. No. 08/655,570, which is incorporated herein by reference. In a representative embodiment, the particles have a mass density less than about 0.4 g/cm³, a mean geometric diameter of between about 5 μm and about 30 μm and MMAD between about 1 μm and about 5 μm.

Suitable particles can be fabricated or separated, for example by filtration or centrifugation, to provide a particle sample with a preselected size distribution. For example, greater than about 30%, 50%, 70%, 80%, 90% or 95% of the particles in a sample can have a diameter within a selected range of at least about 5 μm. The selected range within which a certain percentage of the particles fall may be for example, between about 5 and about 30 μm, or between about 5 and about 15 μm. In one embodiment, at least a portion of the particles have a diameter between about 9 and about 11 μm. Optionally, the particle sample also can be fabricated wherein at least about 75%, 85%, 90%, or optionally about 95% or about 99%, have a diameter within the selected range. The presence of the higher proportion of the aerodynamically light, larger diameter particles in the particle sample can enhance the delivery of therapeutic or diagnostic agents incorporated therein to the deep lung. Large diameter particles generally mean particles having a median geometric diameter of at least about 5 μm.

Properties of the particles facilitate delivery to subjects with highly compromised lungs where other particles prove ineffective for those lacking the capacity to strongly inhale, such as young patients, old subjects, infirm subjects, or subjects with asthma or other breathing difficulties. Further, subjects suffering from a combination of ailments may simply lack the ability to sufficiently inhale. Thus, using the methods and particles described above, even a weak inhalation is sufficient to deliver the desired dose.

Alternatively, in other embodiments, smaller high-density particles that have sufficient momentum to achieve deep lung or alveoli delivery can be used.

Particles can be prepared by any method known in the art. In representative embodiments, suitable particles are fabricated by spray drying. The spray drying can be done under conditions that result in a substantially amorphous powder of homogeneous constitution having a particle size that is respirable, a low moisture content and flow characteristics that allow for ready aerosolization.

In one embodiment, the method includes forming a mixture including one or more compounds of the invention and a surfactant, such as, for example, the surfactants described above. The mixture employed in spray drying can include an organic or aqueous-organic solvent.

Suitable organic solvents that can be employed include but are not limited to alcohols for example, ethanol, methanol, propanol, isopropanol, butanols, and others. Other organic solvents include but are not limited to perfluorocarbons, dichloromethane, chloroform, ether, ethyl acetate, methyl tert-butyl ether and others. Co-solvents include an aqueous solvent and an organic solvent, such as, but not limited to, the organic solvents as described above. Aqueous solvents include water and buffered solutions. In one embodiment, an ethanol water solvent is preferred with the ethanol:water ratio ranging from about 50:50 to about 90:10 ethanol:water.

The spray drying mixture can have a neutral, acidic or alkaline pH (e.g., from about pH 3 to about pH 10). Optionally, a pH buffer can be added to the solvent or co-solvent or to the formed mixture.

Suitable spray-drying techniques are described, for example, by K. Masters in “Spray Drying Handbook”, John Wiley & Sons, New York, 1984. Generally, during spray-drying, heat from a hot gas such as heated air or nitrogen is used to evaporate the solvent from droplets formed by atomizing a continuous liquid feed. Other spray-drying techniques are well known to those skilled in the art. In a preferred embodiment, a rotary atomizer is employed. An example of suitable spray driers using rotary atomization includes the Mobile Minor spray drier, manufactured by Niro, Denmark. The hot gas can be, for example, air, nitrogen or argon.

The particles can be fabricated with a rough surface texture to reduce particle agglomeration and improve flowability of the powder. The spray-dried particles can have improved aerosolization properties. The spray-dried particle can be fabricated with features which enhance aerosolization via dry powder inhaler devices, and lead to lower deposition in the mouth, throat and inhaler device.

Alternatively, dry powder compositions may be prepared by other processes such as lyophilization and jet milling as disclosed in International Patent Publication No. WO 91/16038, the disclosure of which is hereby incorporated by reference.

In other embodiments of the invention, the formulation is administered as a liquid, an emulsion, or a dispersion. Liquid-born agents can be delivered to the lungs by any method known in the art, e.g., by recirculation in and out of the lungs (e.g., by liquid lavage or liquid ventilation) or maintained in a static system (i.e., non-recirculated) for extended periods of time. For example, in representative embodiments, a liquid can be instilled via a lavage tube. As another option, a liquid aerosol can be instilled via a respirator.

U.S. Pat. No. 6,242,472 describes the delivery of therapeutic agents in a liquid carrier such as saline, silicone, vegetable oil or perfluorochemicals (e.g., perfluorocarbon), e.g., in the form of an emulsion or a dispersion, for delivery to the pulmonary air passages; the disclosure of this patent is incorporated by reference herein in its entirety.

The active compound can be present in the liquid in any suitable form, e.g., bulk form, a suspension, a dispersion, a liquid form, an emulsion, and/or an encapsulized form. Moreover, the selected compound(s) can be incorporated into the liquid medium by any suitable technique. Examples of suitable incorporation techniques include, but are not limited to, injection, blending, or dissolving.

Liquids can be selectively directed to specific regions of the subject's lungs by a number of conventional means, such as a bronchoscope or a catheter.

The methods of delivery to the lungs can be carried out once or multiple times, and can further be carried out daily, every other day, etc., with a single administration or multiple administrations per day of administration, (e.g., 2, 3, 4 or more times per day of administration). In representative embodiments, the methods of the invention can be carried out on an as-needed basis by self-medication.

In particular embodiments, the methods of the invention comprise administering to the pulmonary system a therapeutic dose in a small number of breath-activated steps (e.g., less than 5, 4, or 3), and even in one or two breath-activated step(s).

Particular methods include administering particles from a receptacle having, holding, containing, storing or enclosing a mass of particles, to a subject's lungs. In one example, at least 50% of the mass of the particles stored in the inhaler receptacle is delivered to a subject's lungs in a single, breath-activated step. In another embodiment, at least 10 milligrams of the active compound(s) is delivered by administering, in a single breath, to a subject's lungs particles enclosed in the receptacle. Amounts as high as 15, 20, 25, 30, 35, 40 and 50 milligrams or more can be delivered.

In one embodiment, delivery to the pulmonary system of particles in a single, breath-actuated step is enhanced by employing particles that are dispersed at relatively low energies, such as, for example, at energies typically supplied by a subject's inhalation. Such energies are referred to herein as “low.” As used herein, “low energy administration” refers to administration wherein the energy applied to disperse and/or inhale the particles is in the range typically supplied by a subject during inhaling.

The compounds of these embodiments can be pro-drugs that are converted to the active compound in vivo. Further, the activators can be modified to increase their lipophilicity and/or absorption across the nasal mucosa, e.g., by conjugation with lipophilic moieties such as fatty acids.

Some preferred pharmaceutical compositions, for example those to be administered nasally, are in an aqueous solution. Examples of preferred aqueous solutions are aqueous gels, aqueous suspensions, aqueous microsphere suspensions, aqueous microsphere dispersions, aqueous liposomal dispersions, aqueous micelles of liposomes, aqueous microemulsions, and any combinations of the foregoing.

Other preferred pharmaceutical compositions, including those to be nasally administered, are in a nonaqueous solution. Preferred nonaqueous solutions are nonaqueous gels, nonaqueous suspensions, nonaqueous microsphere suspensions, nonaqueous microsphere dispersions, nonaqueous liposomal dispersions, nonaqueous emulsions, nonaqueous microemulsions, and any combination of the foregoing.

Additional preferred pharmaceutical compositions, e.g., those to be nasally administered, are in a powder formulation. Preferred such powder formulations are simple powder mixtures, micronized powders, powder microspheres, coated powder microspheres, and any combination of the foregoing.

The pharmaceutical compositions of these methods are not narrowly limited to any pH range. Optimal pH ranges for any composition and intended purpose can be established by the skilled artisan by established methods. Where the pharmaceutical composition is to be administered nasally, the preferred pH is in the range from pH 3.5 to pH 7.

Similarly, pharmaceutical compositions of these methods are not narrowly limited to any osmolarity range, which can be determined for any composition and intended purpose without undue experimentation. For nasal administration, the osmolarity of the pharmaceutical composition is preferably in the range from 150 to 550 mOsM.

Additionally, where these methods utilize nasal administration, the pharmaceutical composition is preferably in the form of liquid droplets or solid particles, more preferably where the majority and/or mean size of the liquid droplets or solid particles range in size from 5 microns to 50 microns.

Methods utilizing nasal administration preferably also comprise at least one absorption enhancer. These absorption enhancers most preferably comprise a chelating agent or a fatty acid.

The pharmaceutical compositions of the present invention can optionally be administered in conjunction with other therapeutic agents, for example, other therapeutic agents useful in the treatment of hyperglycemia, diabetes, metabolic syndrome and/or obesity. For example, the compounds of the invention can be administered in conjunction with insulin therapy and/or hypoglycemic agents (e.g., metformin). The additional therapeutic agent(s) can be administered concurrently with the compounds of the invention, in the same or different formulations. As used herein, the word “concurrently” means sufficiently close in time to produce a combined effect (that is, concurrently can be simultaneously, or it can be two or more events occurring within a short time period before or after each other).

These methods can be used with any species of mammal, including humans and mammalian species or strains that are experimental models of human disease.

These methods are expected to be useful in conjunction with any other disease treatment or prophylaxis, including but not limited to administration of a second compound, where the second compound is useful for the prevention or treatment of obesity, type 2 diabetes, type 1 diabetes, leptin resistance, insulin resistance, metabolic syndrome, gonadotropin deficiency, amenorrhea, lactic acidosis, and/or polycystic ovary syndrome, or a complication thereof. Nonlimiting examples of such compounds are insulin, antihyperglycemic agents such as troglitazone or metformin, α-glucosidase inhibitors such as acarbose, rennin-angiotensin system blockers such as ramipril, antihypertensive drugs such as ACE inhibitors, angiotensin receptor blockers, β-blockers, diuretics, calcium channel blockers, anti-platelet agents such as clopidogrel, poly (ADP-ribose) polymerase (PARP) inhibitors such as PJ34, 3 aminobenzamide, 4 amino 1,8 naphthalimide, 6(5H) phenanthridinone, benzamide, INO 1001, and NU1025 (see PCT Patent Application No. PCT/US04/16562, filed May 27, 2004), and lipid management agents such as statins, ezetinbe, niacin, fibric acid derivatives, and bile acid sequestrants such as fenofibrate.

The inventors have also discovered that treatments that reduce TCA cycle flux through acetyl-CoA in the hypothalamus of a mammal also cause a reduction in food intake in the mammal. See Example 2.

Thus, the invention is also directed to methods of reducing food intake in a mammal. The methods comprise administering a compound to the mammal, where administering the compound to the mammal causes in increase in tricarboxylic acid (TCA) cycle flux through acetyl-CoA in the hypothalamus of the mammal and a reduction in food intake in the mammal.

In preferred embodiments of these methods, the mammal has a condition that would likely be at least partially alleviated from reduced food intake. Nonlimiting examples of such conditions include obesity, type 2 diabetes, type 1 diabetes, leptin resistance, metabolic syndrome, insulin resistance, gonadotropin deficiency, amenorrhea, and polycystic ovary syndrome.

As with the embodiments described above, the compound can be pyruvate or a metabolic precursor of pyruvate, lactate, a monosaccharide, a disaccharide, or an oligosaccharide. The compound can also increase the activity of an acetyl-CoA-increasing molecule in the hypothalamus of the mammal, where the acetyl-CoA-increasing molecule is preferably a pyruvate dehydrogenase, a lactate dehydrogenase or a pyruvate dehydrogenase phosphatase. In other embodiments, the compound decreases the activity of an acetyl-CoA-decreasing molecule in the hypothalamus of the mammal, where a preferred acetyl-CoA-decreasing molecule is pyruvate dehydrogenase kinase (PDHK). Useful compounds in these embodiments can be a small organic molecule, a nucleic acid, or an oligopeptide or protein, as with the embodiments described above. Examples of PDHK inhibitors are dichloroacetic acid, chlorofluoroacetic acid, difluoroacetic acid, AZD7545, an anilide derivative of (R)-3,3,3-trifluoro-2-hydroxy-2-methylpropionic acid, a secondary amine of (R)-3,3,3-trifluoro-2-hydroxy-2-methylpropionic acid, or an inner lipoyl domain of dihydrolipoyl acetyltransferase.

In other preferred embodiments, the compound is formulated in a pharmaceutical composition that enhances the ability of the compound to cross the blood-brain barrier of the mammal.

Some preferred pharmaceutical compositions, for example those to be administered nasally, are in an aqueous solution. Examples of preferred aqueous solutions are aqueous gels, aqueous suspensions, aqueous microsphere suspensions, aqueous microsphere dispersions, aqueous liposomal dispersions, aqueous micelles of liposomes, aqueous microemulsions, and any combinations of the foregoing.

Other preferred pharmaceutical compositions, including those to be nasally administered, are in a nonaqueous solution. Preferred nonaqueous solutions are nonaqueous gels, nonaqueous suspensions, nonaqueous microsphere suspensions, nonaqueous microsphere dispersions, nonaqueous liposomal dispersions, nonaqueous emulsions, nonaqueous microemulsions, and any combination of the foregoing.

Additional preferred pharmaceutical compositions, e.g., those to be nasally administered, are in a powder formulation. Preferred such powder formulations are simple powder mixtures, micronized powders, powder microspheres, coated powder microspheres, and any combination of the foregoing.

The pharmaceutical compositions of these methods are not narrowly limited to any pH range. Optimal pH ranges for any composition and intended purpose can be established by the skilled artisan by established methods. Where the pharmaceutical composition is to be administered nasally, the preferred pH is in the range from pH 3.5 to pH 7.

Similarly, pharmaceutical compositions of these methods are not narrowly limited to any osmolarity range, which can be determined for any composition and intended purpose without undue experimentation. For nasal administration, the osmolarity of the pharmaceutical composition is preferably in the range from 150 to 550 mOsM.

Additionally, where these methods utilize nasal administration, the pharmaceutical composition is preferably in the form of liquid droplets or solid particles, more preferably where the majority and/or mean size of the liquid droplets or solid particles range in size from 5 microns to 50 microns.

Methods utilizing nasal administration preferably also comprise at least one absorption enhancer. These absorption enhancers most preferably comprise a chelating agent or a fatty acid.

These methods can be used with any species of mammal, including humans and mammalian species or strains that are experimental models of human disease.

These methods are also expected to be useful in conjunction with any other disease treatment or prophylaxis, including but not limited to administration of a second compound, where the second compound is useful for the prevention or treatment of obesity, type 2 diabetes, type 1 diabetes, leptin resistance, insulin resistance, metabolic syndrome, gonadotropin deficiency, amenorrhea, lactic acidosis, and/or polycystic ovary syndrome, or a complication thereof. Nonlimiting examples of such compounds are insulin, antihyperglycemic agents such as troglitazone or metformin, α-glucosidase inhibitors such as acarbose, rennin-angiotensin system blockers such as ramipril, antihypertensive drugs such as ACE inhibitors, angiotensin receptor blockers, β-blockers, diuretics, calcium channel blockers, anti-platelet agents such as clopidogrel, poly (ADP-ribose) polymerase (PARP) inhibitors such as PJ34, 3 aminobenzamide, 4 amino 1,8 naphthalimide, 6(5H) phenanthridinone, benzamide, INO 1001, and NU1025 (see PCT Patent Application No. PCT/US04/16562, filed May 27, 2004), and lipid management agents such as statins, ezetinbe, niacin, fibric acid derivatives, and bile acid sequestrants such as fenofibrate.

The inventors have also determined that the reduced glucose production and peripheral blood glucose levels caused by the increasing TCA cycle flux through acetyl-CoA is due primarily to a decrease in liver gluconeogenesis. See Example 1. Thus, in additional embodiments, the invention is directed to methods of inhibiting gluconeogenesis in the liver of a mammal. The methods comprise administering a compound to the mammal, where administering the compound to the mammal causes an increase in tricarboxylic acid (TCA) cycle flux through acetyl-CoA in the hypothalamus of the mammal and an inhibition of gluconeogenesis in the mammal. In preferred embodiments of these methods, the mammal has a condition that would likely be at least partially alleviated from reduced liver gluconeogenesis. Nonlimiting examples of such conditions include obesity, type 2 diabetes, type 1 diabetes, leptin resistance, metabolic syndrome, insulin resistance, gonadotropin deficiency, amenorrhea, and polycystic ovary syndrome.

As with the embodiments described above, the compound can be pyruvate or a metabolic precursor of pyruvate, lactate, a monosaccharide, a disaccharide, or an oligosaccharide. The compound can also increase the activity of an acetyl-CoA-increasing molecule in the hypothalamus of the mammal, where the acetyl-CoA-increasing molecule is preferably a pyruvate dehydrogenase, a lactate dehydrogenase or a pyruvate dehydrogenase phosphatase. In other embodiments, the compound decreases the activity of an acetyl-CoA-decreasing molecule in the hypothalamus of the mammal, where a preferred acetyl-CoA-decreasing molecule is pyruvate dehydrogenase kinase (PDHK). Useful compounds in these embodiments can be a small organic molecule, a nucleic acid, or an oligopeptide or protein, as with the embodiments described above. Examples of PDHK inhibitors are dichloroacetic acid, chlorofluoroacetic acid, difluoroacetic acid, AZD7545, an anilide derivative of (R)-3,3,3-trifluoro-2-hydroxy-2-methylpropionic acid, a secondary amine of (R)-3,3,3-trifluoro-2-hydroxy-2-methylpropionic acid, or an inner lipoyl domain of dihydrolipoyl acetyltransferase.

In other preferred embodiments, the compound is formulated in a pharmaceutical composition that enhances the ability of the compound to cross the blood-brain barrier of the mammal.

Some preferred pharmaceutical compositions, for example those to be administered nasally, are in an aqueous solution. Examples of preferred aqueous solutions are aqueous gels, aqueous suspensions, aqueous microsphere suspensions, aqueous microsphere dispersions, aqueous liposomal dispersions, aqueous micelles of liposomes, aqueous microemulsions, and any combinations of the foregoing.

Other preferred pharmaceutical compositions, including those to be nasally administered, are in a nonaqueous solution. Preferred nonaqueous solutions are nonaqueous gels, nonaqueous suspensions, nonaqueous microsphere suspensions, nonaqueous microsphere dispersions, nonaqueous liposomal dispersions, nonaqueous emulsions, nonaqueous microemulsions, and any combination of the foregoing.

Additional preferred pharmaceutical compositions, e.g., those to be nasally administered, are in a powder formulation. Preferred such powder formulations are simple powder mixtures, micronized powders, powder microspheres, coated powder microspheres, and any combination of the foregoing.

The pharmaceutical compositions of these methods are not narrowly limited to any pH range. Optimal pH ranges for any composition and intended purpose can be established by the skilled artisan by established methods. Where the pharmaceutical composition is to be administered nasally, the preferred pH is in the range from pH 3.5 to pH 7.

Similarly, pharmaceutical compositions of these methods are not narrowly limited to any osmolarity range, which can be determined for any composition and intended purpose without undue experimentation. For nasal administration, the osmolarity of the pharmaceutical composition is preferably in the range from 150 to 550 mOsM.

Additionally, where these methods utilize nasal administration, the pharmaceutical composition is preferably in the form of liquid droplets or solid particles, more preferably where the majority and/or mean size of the liquid droplets or solid particles range in size from 5 microns to 50 microns.

Methods utilizing nasal administration preferably also comprise at least one absorption enhancer. These absorption enhancers most preferably comprise a chelating agent or a fatty acid.

These methods can be used with any species of mammal, including humans and mammalian species or strains that are experimental models of human disease.

In further embodiments, the invention is directed to methods of reducing peripheral blood glucose levels in a mammal. The methods comprise administering a compound to the mammal, where administering the compound to the mammal causes an increase in tricarboxylic acid (TCA) cycle flux through acetyl-CoA in the hypothalamus of the mammal and a reduction in peripheral blood glucose levels in the mammal.

In preferred embodiments of these methods, the mammal has a condition that would likely be at least partially alleviated from reduced peripheral blood glucose levels. Nonlimiting examples of such conditions include obesity, type 2 diabetes, type 1 diabetes, leptin resistance, metabolic syndrome, insulin resistance, gonadotropin deficiency, amenorrhea, and polycystic ovary syndrome.

As with the embodiments described above, the compound can be pyruvate or a metabolic precursor of pyruvate, lactate, a monosaccharide, a disaccharide, or an oligosaccharide. The compound can also increase the activity of an acetyl-CoA-increasing molecule in the hypothalamus of the mammal, where the acetyl-CoA-increasing molecule is preferably a pyruvate dehydrogenase, a lactate dehydrogenase or a pyruvate dehydrogenase phosphatase. In other embodiments, the compound decreases the activity of an acetyl-CoA-decreasing molecule in the hypothalamus of the mammal, where a preferred acetyl-CoA-decreasing molecule is pyruvate dehydrogenase kinase (PDHK). Useful compounds in these embodiments can be a small organic molecule, a nucleic acid, or an oligopeptide or protein, as with the embodiments described above. Examples of PDHK inhibitors are dichloroacetic acid, chlorofluoroacetic acid, difluoroacetic acid, AZD7545, an anilide derivative of (R)-3,3,3-trifluoro-2-hydroxy-2-methylpropionic acid, a secondary amine of (R)-3,3,3-trifluoro-2-hydroxy-2-methylpropionic acid, or an inner lipoyl domain of dihydrolipoyl acetyltransferase.

In other preferred embodiments, the compound is formulated in a pharmaceutical composition that enhances the ability of the compound to cross the blood-brain barrier of the mammal.

Some preferred pharmaceutical compositions, for example those to be administered nasally, are in an aqueous solution. Examples of preferred aqueous solutions are aqueous gels, aqueous suspensions, aqueous microsphere suspensions, aqueous microsphere dispersions, aqueous liposomal dispersions, aqueous micelles of liposomes, aqueous microemulsions, and any combinations of the foregoing.

Other preferred pharmaceutical compositions, including those to be nasally administered, are in a nonaqueous solution. Preferred nonaqueous solutions are nonaqueous gels, nonaqueous suspensions, nonaqueous microsphere suspensions, nonaqueous microsphere dispersions, nonaqueous liposomal dispersions, nonaqueous emulsions, nonaqueous microemulsions, and any combination of the foregoing.

Additional preferred pharmaceutical compositions, e.g., those to be nasally administered, are in a powder formulation. Preferred such powder formulations are simple powder mixtures, micronized powders, powder microspheres, coated powder microspheres, and any combination of the foregoing.

The pharmaceutical compositions of these methods are not narrowly limited to any pH range. Optimal pH ranges for any composition and intended purpose can be established by the skilled artisan by established methods. Where the pharmaceutical composition is to be administered nasally, the preferred pH is in the range from pH 3.5 to pH 7.

Similarly, pharmaceutical compositions of these methods are not narrowly limited to any osmolarity range, which can be determined for any composition and intended purpose without undue experimentation. For nasal administration, the osmolarity of the pharmaceutical composition is preferably in the range from 150 to 550 mOsM.

Additionally, where these methods utilize nasal administration, the pharmaceutical composition is preferably in the form of liquid droplets or solid particles, more preferably where the majority and/or mean size of the liquid droplets or solid particles range in size from 5 microns to 50 microns.

Methods utilizing nasal administration preferably also comprise at least one absorption enhancer. These absorption enhancers most preferably comprise a chelating agent or a fatty acid.

These methods can be used with any species of mammal, including humans and mammalian species or strains that are experimental models of human disease.

These methods are also expected to be useful in conjunction with any other disease treatment or prophylaxis, including but not limited to administration of a second compound, where the second compound is useful for the prevention or treatment of obesity, type 2 diabetes, type 1 diabetes, leptin resistance, insulin resistance, metabolic syndrome, gonadotropin deficiency, amenorrhea, lactic acidosis, and/or polycystic ovary syndrome, or a complication thereof. Nonlimiting examples of such compounds are insulin, antihyperglycemic agents such as troglitazone or metformin, α-glucosidase inhibitors such as acarbose, rennin-angiotensin system blockers such as ramipril, antihypertensive drugs such as ACE inhibitors, angiotensin receptor blockers, β-blockers, diuretics, calcium channel blockers, anti-platelet agents such as clopidogrel, poly (ADP-ribose) polymerase (PARP) inhibitors such as PJ34, 3 aminobenzamide, 4 amino 1,8 naphthalimide, 6(5H) phenanthridinone, benzamide, INO 1001, and NU1025 (see PCT Patent Application No. PCT/US04/16562, filed May 27, 2004), and lipid management agents such as statins, ezetinbe, niacin, fibric acid derivatives, and bile acid sequestrants such as fenofibrate.

The inventors have also discovered that increasing TCA cycle flux through acetyl-CoA in the hypothalamus of a mammal causes a decrease in serum triglycerides and very low density lipoprotein (VLDL). Since reduction of serum triglycerides and VLDL is beneficial, particularly for cardiovascular health, the invention methods can be used as a treatment for high triglyceride and/or VLDL levels.

The invention is therefore also directed to methods of decreasing serum triglyceride levels in a mammal. The methods comprise administering a compound to the mammal, where administering the compound to the mammal causes an increase in tricarboxylic acid (TCA) cycle flux through acetyl-CoA in the hypothalamus of the mammal and a decrease in serum triglyceride levels in the mammal.

In preferred embodiments of these methods, the mammal has a condition that would likely be at least partially alleviated from reduced serum triglyceride levels. Nonlimiting examples of such conditions include obesity, type 2 diabetes, type 1 diabetes, leptin resistance, insulin resistance, metabolic syndrome, gonadotropin deficiency, amenorrhea, polycystic ovary syndrome, familial lipoprotein lipase deficiency, coronary heart disease, atherosclerosis, hypopituitarism, hypercholesterolemia, hyperlipidemia, and hypertriglyceridemia.

As with the embodiments described above, the compound can be pyruvate or a metabolic precursor of pyruvate, lactate, a monosaccharide, a disaccharide, or an oligosaccharide. The compound can also increase the activity of an acetyl-CoA-increasing molecule in the hypothalamus of the mammal, where the acetyl-CoA-increasing molecule is preferably a pyruvate dehydrogenase, a lactate dehydrogenase or a pyruvate dehydrogenase phosphatase. In other embodiments, the compound decreases the activity of an acetyl-CoA-decreasing molecule in the hypothalamus of the mammal, where a preferred acetyl-CoA-decreasing molecule is pyruvate dehydrogenase kinase (PDHK). Useful compounds in these embodiments can be a small organic molecule, a nucleic acid, or an oligopeptide or protein, as with the embodiments described above. Examples of PDHK inhibitors are dichloroacetic acid, chlorofluoroacetic acid, difluoroacetic acid, AZD7545, an anilide derivative of (R)-3,3,3-trifluoro-2-hydroxy-2-methylpropionic acid, a secondary amine of (R)-3,3,3-trifluoro-2-hydroxy-2-methylpropionic acid, or an inner lipoyl domain of dihydrolipoyl acetyltransferase.

In other preferred embodiments, the compound is formulated in a pharmaceutical composition that enhances the ability of the compound to cross the blood-brain barrier of the mammal.

Some preferred pharmaceutical compositions, for example those to be administered nasally, are in an aqueous solution. Examples of preferred aqueous solutions are aqueous gels, aqueous suspensions, aqueous microsphere suspensions, aqueous microsphere dispersions, aqueous liposomal dispersions, aqueous micelles of liposomes, aqueous microemulsions, and any combinations of the foregoing.

Other preferred pharmaceutical compositions, including those to be nasally administered, are in a nonaqueous solution. Preferred nonaqueous solutions are nonaqueous gels, nonaqueous suspensions, nonaqueous microsphere suspensions, nonaqueous microsphere dispersions, nonaqueous liposomal dispersions, nonaqueous emulsions, nonaqueous microemulsions, and any combination of the foregoing.

Additional preferred pharmaceutical compositions, e.g., those to be nasally administered, are in a powder formulation. Preferred such powder formulations are simple powder mixtures, micronized powders, powder microspheres, coated powder microspheres, and any combination of the foregoing.

The pharmaceutical compositions of these methods are not narrowly limited to any pH range. Optimal pH ranges for any composition and intended purpose can be established by the skilled artisan by established methods. Where the pharmaceutical composition is to be administered nasally, the preferred pH is in the range from pH 3.5 to pH 7.

Similarly, pharmaceutical compositions of these methods are not narrowly limited to any osmolarity range, which can be determined for any composition and intended purpose without undue experimentation. For nasal administration, the osmolarity of the pharmaceutical composition is preferably in the range from 150 to 550 mOsM.

Additionally, where these methods utilize nasal administration, the pharmaceutical composition is preferably in the form of liquid droplets or solid particles, more preferably where the majority and/or mean size of the liquid droplets or solid particles range in size from 5 microns to 50 microns.

Methods utilizing nasal administration preferably also comprise at least one absorption enhancer. These absorption enhancers most preferably comprise a chelating agent or a fatty acid.

These methods can be used with any species of mammal, including humans and mammalian species or strains that are experimental models of human disease.

These methods are also expected to be useful in conjunction with any other disease treatment or prophylaxis, including but not limited to administration of a second compound, where the second compound is useful for the prevention or treatment of obesity, type 2 diabetes, type 1 diabetes, leptin resistance, insulin resistance, metabolic syndrome, gonadotropin deficiency, amenorrhea, lactic acidosis, and/or polycystic ovary syndrome, or a complication thereof. Nonlimiting examples of such compounds are insulin, antihyperglycemic agents such as troglitazone or metformin, α-glucosidase inhibitors such as acarbose, rennin-angiotensin system blockers such as ramipril, antihypertensive drugs such as ACE inhibitors, angiotensin receptor blockers, β-blockers, diuretics, calcium channel blockers, anti-platelet agents such as clopidogrel, poly (ADP-ribose) polymerase (PARP) inhibitors such as PJ34, 3 aminobenzamide, 4 amino 1,8 naphthalimide, 6(5H) phenanthridinone, benzamide, INO 1001, and NU1025 (see PCT Patent Application No. PCT/US04/16562, filed May 27, 2004), and lipid management agents such as statins, ezetinbe, niacin, fibric acid derivatives, and bile acid sequestrants such as fenofibrate.

The invention is additionally directed to methods of decreasing very low density lipoprotein (VLDL) levels in a mammal. The methods comprise administering a compound to the mammal, where administering the compound to the mammal causes an increase in tricarboxylic acid (TCA) cycle flux through acetyl-CoA in the hypothalamus of the mammal and a decrease in VLDL levels in the mammal.

In preferred embodiments of these methods, the mammal has a condition that would likely be at least partially alleviated from reduced VLDL levels. Nonlimiting examples of such conditions include obesity, type 2 diabetes, type 1 diabetes, leptin resistance, insulin resistance, metabolic syndrome, gonadotropin deficiency, amenorrhea, polycystic ovary syndrome, familial lipoprotein lipase deficiency, coronary heart disease, atherosclerosis, hypopituitarism, hypercholesterolemia, hyperlipidemia, and hypertriglyceridemia.

As with the embodiments described above, the compound can be pyruvate or a metabolic precursor of pyruvate, lactate, a monosaccharide, a disaccharide, or an oligosaccharide. The compound can also increase the activity of an acetyl-CoA-increasing molecule in the hypothalamus of the mammal, where the acetyl-CoA-increasing molecule is preferably a pyruvate dehydrogenase, a lactate dehydrogenase or a pyruvate dehydrogenase phosphatase. In other embodiments, the compound decreases the activity of an acetyl-CoA-decreasing molecule in the hypothalamus of the mammal, where a preferred acetyl-CoA-decreasing molecule is pyruvate dehydrogenase kinase (PDHK). Useful compounds in these embodiments can be a small organic molecule, a nucleic acid, or an oligopeptide or protein, as with the embodiments described above. Examples of PDHK inhibitors are dichloroacetic acid, chlorofluoroacetic acid, difluoroacetic acid, AZD7545, an anilide derivative of (R)-3,3,3-trifluoro-2-hydroxy-2-methylpropionic acid, a secondary amine of (R)-3,3,3-trifluoro-2-hydroxy-2-methylpropionic acid, or an inner lipoyl domain of dihydrolipoyl acetyltransferase.

In other preferred embodiments, the compound is formulated in a pharmaceutical composition that enhances the ability of the compound to cross the blood-brain barrier of the mammal.

Some preferred pharmaceutical compositions, for example those to be administered nasally, are in an aqueous solution. Examples of preferred aqueous solutions are aqueous gels, aqueous suspensions, aqueous microsphere suspensions, aqueous microsphere dispersions, aqueous liposomal dispersions, aqueous micelles of liposomes, aqueous micro emulsions, and any combinations of the foregoing.

Other preferred pharmaceutical compositions, including those to be nasally administered, are in a nonaqueous solution. Preferred nonaqueous solutions are nonaqueous gels, nonaqueous suspensions, nonaqueous microsphere suspensions, nonaqueous microsphere dispersions, nonaqueous liposomal dispersions, nonaqueous emulsions, nonaqueous microemulsions, and any combination of the foregoing.

Additional preferred pharmaceutical compositions, e.g., those to be nasally administered, are in a powder formulation. Preferred such powder formulations are simple powder mixtures, micronized powders, powder microspheres, coated powder microspheres, and any combination of the foregoing.

The pharmaceutical compositions of these methods are not narrowly limited to any pH range. Optimal pH ranges for any composition and intended purpose can be established by the skilled artisan by established methods. Where the pharmaceutical composition is to be administered nasally, the preferred pH is in the range from pH 3.5 to pH 7.

Similarly, pharmaceutical compositions of these methods are not narrowly limited to any osmolarity range, which can be determined for any composition and intended purpose without undue experimentation. For nasal administration, the osmolarity of the pharmaceutical composition is preferably in the range from 150 to 550 mOsM.

Additionally, where these methods utilize nasal administration, the pharmaceutical composition is preferably in the form of liquid droplets or solid particles, more preferably where the majority and/or mean size of the liquid droplets or solid particles range in size from 5 microns to 50 microns.

Methods utilizing nasal administration preferably also comprise at least one absorption enhancer. These absorption enhancers most preferably comprise a chelating agent or a fatty acid.

These methods can be used with any species of mammal, including humans and mammalian species or strains that are experimental models of human disease.

The inventors have also discovered that glucose production can be increased in a mammal by causing a decrease in TCA cycle flux through acetyl-CoA Thus, the invention is additionally directed to methods of increasing glucose production in a mammal. The methods comprise administering a compound to the mammal, where administering the compound to the mammal causes a decrease in tricarboxylic acid (TCA) cycle flux through acetyl-CoA in the hypothalamus of the mammal and an increase in glucose production in the mammal.

These methods are useful in any situation where it is desired that the mammal increase its food intake or glucose production. Examples of such situations are when the mammal is undergoing a treatment that causes insufficient food intake or glucose production, for example cancer chemotherapy, or when the mammal has an infection, such as a viral infection (e.g., HIV-1 infection) that causes insufficient food intake or glucose production. The methods are also effective in mammals that are hypoglycemic.

Analogous to the embodiments described above, one way of causing a decrease in TCA cycle flux through acetyl-CoA in the hypothalamus is to add a compound to the hypothalamus, where the compound increases the activity of an acetyl-CoA-decreasing molecule in the hypothalamus of the mammal. As discussed above, an example of an acetyl-CoA-decreasing molecule is PDHK.

Another way of causing a decrease in TCA cycle flux through acetyl-CoA in the hypothalamus is to add a compound to the hypothalamus, where the compound decreases the activity of an acetyl-CoA-increasing molecule in the hypothalamus of the mammal. Also as discussed above, examples of acetyl-CoA-increasing molecules are PDH, LDH, and pyruvate dehydrogenase phosphatase.

Preferably the compounds in these embodiments are small organic molecules, for example small organic molecule inhibitor of PDH, such as 3-bromopyruvate, thiamine thiazolone, or thiamine thiazolone pyrophosphate, a small organic molecule inhibitor of LDH, such as oxamate. The compound can also be an antibody or aptamer that specifically inhibits the acetyl-CoA increasing molecule.

In other preferred embodiments, the compound is formulated in a pharmaceutical composition that enhances the ability of the compound to cross the blood-brain barrier of the mammal. Also as with embodiments disclosed above, the compound can be administered directly to the brain of the mammal.

Some preferred pharmaceutical compositions, for example those to be administered nasally, are in an aqueous solution. Examples of preferred aqueous solutions are aqueous gels, aqueous suspensions, aqueous microsphere suspensions, aqueous microsphere dispersions, aqueous liposomal dispersions, aqueous micelles of liposomes, aqueous micro emulsions, and any combinations of the foregoing.

Other preferred pharmaceutical compositions, including those to be nasally administered, are in a nonaqueous solution. Preferred nonaqueous solutions are nonaqueous gels, nonaqueous suspensions, nonaqueous microsphere suspensions, nonaqueous microsphere dispersions, nonaqueous liposomal dispersions, nonaqueous emulsions, nonaqueous microemulsions, and any combination of the foregoing.

Additional preferred pharmaceutical compositions, e.g., those to be nasally administered, are in a powder formulation. Preferred such powder formulations are simple powder mixtures, micronized powders, powder microspheres, coated powder microspheres, and any combination of the foregoing.

The pharmaceutical compositions of these methods are not narrowly limited to any pH range. Optimal pH ranges for any composition and intended purpose can be established by the skilled artisan by established methods. Where the pharmaceutical composition is to be administered nasally, the preferred pH is in the range from pH 3.5 to pH 7.

Similarly, pharmaceutical compositions of these methods are not narrowly limited to any osmolarity range, which can be determined for any composition and intended purpose without undue experimentation. For nasal administration, the osmolarity of the pharmaceutical composition is preferably in the range from 150 to 550 mOsM.

Additionally, where these methods utilize nasal administration, the pharmaceutical composition is preferably in the form of liquid droplets or solid particles, more preferably where the majority and/or mean size of the liquid droplets or solid particles range in size from 5 microns to 50 microns.

Methods utilizing nasal administration preferably also comprise at least one absorption enhancer. These absorption enhancers most preferably comprise a chelating agent or a fatty acid.

These methods can be used with any species of mammal, including humans and mammalian species or strains that are experimental models of human disease.

The invention is also directed to methods of increasing food intake in a mammal. The methods comprise administering a compound to the mammal, where administering the compound to the mammal causes a decrease in tricarboxylic acid (TCA) cycle flux through acetyl-CoA in the hypothalamus of the mammal and an increase in food intake in the mammal.

These methods are useful in any situation where it is desired that the mammal increase its food intake or glucose production. Examples of such situations are when the mammal is undergoing a treatment that causes insufficient food intake or glucose production, for example cancer chemotherapy, or when the mammal has an infection, such as a viral infection (e.g., HIV-1 infection) that causes insufficient food intake or glucose production. The methods are also effective in mammals that are hypoglycemic.

As discussed above, one way of causing a decrease in TCA cycle flux through acetyl-CoA in the hypothalamus is to add a compound to the hypothalamus, where the compound increases the activity of an acetyl-CoA-decreasing molecule in the hypothalamus of the mammal. Also as discussed above, an example of an acetyl-CoA-decreasing molecule is PDHK.

Another way of causing a decrease in TCA cycle flux through acetyl-CoA in the hypothalamus is to add a compound to the hypothalamus, where the compound decreases the activity of an acetyl-CoA-increasing molecule in the hypothalamus of the mammal. Also as discussed above, examples of acetyl-CoA-increasing molecules are PDH, LDH, and pyruvate dehydrogenase phosphatase.

Preferably the compounds in these embodiments are small organic molecules, for example small organic molecule inhibitor of PDH, such as 3-bromopyruvate, thiamine thiazolone, or thiamine thiazolone pyrophosphate, or a small organic molecule inhibitor of LDH, such as oxamate. The compound can also be an antibody or aptamer that specifically inhibits the acetyl-CoA increasing molecule.

In other preferred embodiments, the compound is formulated in a pharmaceutical composition that enhances the ability of the compound to cross the blood-brain barrier of the mammal. Also as with embodiments disclosed above, the compound can be administered directly to the brain of the mammal.

Some preferred pharmaceutical compositions, for example those to be administered nasally, are in an aqueous solution. Examples of preferred aqueous solutions are aqueous gels, aqueous suspensions, aqueous microsphere suspensions, aqueous microsphere dispersions, aqueous liposomal dispersions, aqueous micelles of liposomes, aqueous microemulsions, and any combinations of the foregoing.

Other preferred pharmaceutical compositions, including those to be nasally administered, are in a nonaqueous solution. Preferred nonaqueous solutions are nonaqueous gels, nonaqueous suspensions, nonaqueous microsphere suspensions, nonaqueous microsphere dispersions, nonaqueous liposomal dispersions, nonaqueous emulsions, nonaqueous microemulsions, and any combination of the foregoing.

Additional preferred pharmaceutical compositions, e.g., those to be nasally administered, are in a powder formulation. Preferred such powder formulations are simple powder mixtures, micronized powders, powder microspheres, coated powder microspheres, and any combination of the foregoing.

The pharmaceutical compositions of these methods are not narrowly limited to any pH range. Optimal pH ranges for any composition and intended purpose can be established by the skilled artisan by established methods. Where the pharmaceutical composition is to be administered nasally, the preferred pH is in the range from pH 3.5 to pH 7.

Similarly, pharmaceutical compositions of these methods are not narrowly limited to any osmolarity range, which can be determined for any composition and intended purpose without undue experimentation. For nasal administration, the osmolarity of the pharmaceutical composition is preferably in the range from 150 to 550 mOsM.

Additionally, where these methods utilize nasal administration, the pharmaceutical composition is preferably in the form of liquid droplets or solid particles, more preferably where the majority and/or mean size of the liquid droplets or solid particles range in size from 5 microns to 50 microns.

Methods utilizing nasal administration preferably also comprise at least one absorption enhancer. These absorption enhancers most preferably comprise a chelating agent or a fatty acid.

These methods can be used with any species of mammal, including humans and mammalian species or strains that are experimental models of human disease.

These methods are also expected to be useful in conjunction with any other disease treatment or prophylaxis, including but not limited to administration of a second compound, where the second compound is useful for the prevention or treatment of obesity, type 2 diabetes, type 1 diabetes, leptin resistance, insulin resistance, metabolic syndrome, gonadotropin deficiency, amenorrhea, lactic acidosis, and/or polycystic ovary syndrome, or a complication thereof. Nonlimiting examples of such compounds are insulin, antihyperglycemic agents such as troglitazone or metformin, α-glucosidase inhibitors such as acarbose, rennin-angiotensin system blockers such as ramipril, antihypertensive drugs such as ACE inhibitors, angiotensin receptor blockers, β-blockers, diuretics, calcium channel blockers, anti-platelet agents such as clopidogrel, poly (ADP-ribose) polymerase (PARP) inhibitors such as PJ34, 3 aminobenzamide, 4 amino 1,8 naphthalimide, 6(5H) phenanthridinone, benzamide, INO 1001, and NU1025 (see PCT Patent Application No. PCT/US04/16562, filed May 27, 2004), and lipid management agents such as statins, ezetinbe, niacin, fibric acid derivatives, and bile acid sequestrants such as fenofibrate.

The inventors have also realized that, given the discovery that increasing TCA cycle flux through acetyl-CoA in the hypothalamus causes a decrease in triglyceride and VLDL levels, other hypothalamic manipulations that cause reduced glucose production and peripheral blood glucose levels (described in PCT Patent Application No. PCT/US2004/004344) would also cause a decrease in triglyceride and VLDL levels.

Thus, the invention is also directed to other methods of decreasing very low density lipoprotein (VLDL) levels in a mammal. The methods comprise increasing long-chain fatty acyl-Co-A (LC-CoA) levels in the hypothalamus of the mammal (see PCT Patent Application No. PCT/US2004/004344) in an amount effective to reduce VLDL levels in the mammal.

As in embodiments described above, the mammal preferably has a condition that would benefit from decreases in VLDL levels. Nonlimiting examples include obesity, type 2 diabetes, type 1 diabetes, leptin resistance, insulin resistance, metabolic syndrome, gonadotropin deficiency, amenorrhea, polycystic ovary syndrome, familial lipoprotein lipase deficiency, coronary heart disease, atherosclerosis, hypopituitarism, hypercholesterolemia, hyperlipidemia, and hypertriglyceridemia. As discussed in PCT Patent Application PCT/US2004/004344, the LC-CoA levels can be increased by decreasing the activity of an LC-CoA-decreasing molecule in the hypothalamus. Examples of LC-CoA-decreasing molecules are carnitine palmitoyl transferase 1 (CPT1), malonyl-CoA decarboxylase, carnitine acylcarnitine translocase, acyl-CoA dehydrogenase, 2-enoyl-CoA hydratase, 3-hydroxyacyl-CoA dehydrogenase, 3-oxyacyl-CoA thiolase, and acyl-CoA hydrolase. Preferably, the LC-CoA-decreasing molecule is CPT1, most preferably carnitine palmitoyl transferase-1, liver isoform (CPT1L).

The activity of the LC-CoA-decreasing molecule can be decreased by administering a pharmaceutical composition to the brain of the mammal, where the pharmaceutical composition comprises a small molecule capable of decreasing the activity of the LC-CoA-decreasing molecule. The pharmaceutical composition can be administered to the brain of the mammal by direct administration of the composition to the brain. Alternatively, the small molecule is capable of crossing the blood-brain barrier when present in the pharmaceutical composition.

In other embodiments, the activity of the LC-CoA-decreasing molecule is decreased by administering an antibody or antibody fragment comprising an antibody binding site to the brain of the mammal, where the antibody or antibody fragment is capable of binding to the LC-CoA-decreasing molecule to inhibit the activity of the molecule. Alternatively, the activity of the LC-CoA-decreasing molecule can be decreased by administering an inhibitory nucleic acid or mimetic to the brain of the mammal. Nonlimiting examples of inhibitory nucleic acids or mimetics are ribozymes, antisense compounds, aptamers and iRNAs. In preferred embodiments, the inhibitory nucleic acid or mimetic is a ribozyme, e.g., where the LC-CoA-decreasing molecule is CPT1L and the ribozyme comprises the sequence 5′-ACAGCACGCCGCUCUGAUGAGUCCGUGAGGACGAAACCACGUUCUUCGUC-3′.

In other embodiments, the LC-CoA levels are increased by increasing the activity of an LC-CoA-increasing molecule in the hypothalamus. Examples of LC-CoA-increasing molecules are acetyl-CoA carboxylase, fatty acid transporter molecule, acyl-CoA synthetase, carnitine palmitoyl transferase II, and acyl-CoA thioesterase.

In these embodiments, the activity of the LC-CoA-increasing molecule can be increased by administering a pharmaceutical composition to the brain of the mammal, where the pharmaceutical composition comprises a small molecule capable of stimulating production or activity of the LC-CoA-increasing molecule. Here, the pharmaceutical composition can be administered to the brain of the mammal by direct administration to the brain. Alternatively, the the pharmaceutical composition is capable of crossing the blood-brain barrier.

The activity of the LC-CoA-increasing molecule can also be increased by administering the molecule or a vector (e.g., a viral vector) encoding the molecule to the hypothalamus, e.g., in a pharmaceutically acceptable carrier. The LC-CoA can also be increased by directly administering LC-CoA to the brain.

Some preferred pharmaceutical compositions, for example those to be administered nasally, are in an aqueous solution. Examples of preferred aqueous solutions are aqueous gels, aqueous suspensions, aqueous microsphere suspensions, aqueous microsphere dispersions, aqueous liposomal dispersions, aqueous micelles of liposomes, aqueous microemulsions, and any combinations of the foregoing.

Other preferred pharmaceutical compositions, including those to be nasally administered, are in a nonaqueous solution. Preferred nonaqueous solutions are nonaqueous gels, nonaqueous suspensions, nonaqueous microsphere suspensions, nonaqueous microsphere dispersions, nonaqueous liposomal dispersions, nonaqueous emulsions, nonaqueous microemulsions, and any combination of the foregoing.

Additional preferred pharmaceutical compositions, e.g., those to be nasally administered, are in a powder formulation. Preferred such powder formulations are simple powder mixtures, micronized powders, powder microspheres, coated powder microspheres, and any combination of the foregoing.

The pharmaceutical compositions of these methods are not narrowly limited to any pH range. Optimal pH ranges for any composition and intended purpose can be established by the skilled artisan by established methods. Where the pharmaceutical composition is to be administered nasally, the preferred pH is in the range from pH 3.5 to pH 7.

Similarly, pharmaceutical compositions of these methods are not narrowly limited to any osmolarity range, which can be determined for any composition and intended purpose without undue experimentation. For nasal administration, the osmolarity of the pharmaceutical composition is preferably in the range from 150 to 550 mOsM.

Additionally, where these methods utilize nasal administration, the pharmaceutical composition is preferably in the form of liquid droplets or solid particles, more preferably where the majority and/or mean size of the liquid droplets or solid particles range in size from 5 microns to 50 microns.

Methods utilizing nasal administration preferably also comprise at least one absorption enhancer. These absorption enhancers most preferably comprise a chelating agent or a fatty acid.

These methods are also expected to be useful in conjunction with any other disease treatment or prophylaxis, including but not limited to administration of a second compound, where the second compound is useful for the prevention or treatment of obesity, type 2 diabetes, type 1 diabetes, leptin resistance, insulin resistance, metabolic syndrome, gonadotropin deficiency, amenorrhea, lactic acidosis, and/or polycystic ovary syndrome, or a complication thereof. Nonlimiting examples of such compounds are insulin, antihyperglycemic agents such as troglitazone or metformin, α-glucosidase inhibitors such as acarbose, rennin-angiotensin system blockers such as ramipril, antihypertensive drugs such as ACE inhibitors, angiotensin receptor blockers, β-blockers, diuretics, calcium channel blockers, anti-platelet agents such as clopidogrel, poly (ADP-ribose) polymerase (PARP) inhibitors such as PJ34, 3 aminobenzamide, 4 amino 1,8 naphthalimide, 6(5H) phenanthridinone, benzamide, INO 1001, and NU1025 (see PCT Patent Application No. PCT/US04/16562, filed May 27, 2004), and lipid management agents such as statins, ezetinbe, niacin, fibric acid derivatives, and bile acid sequestrants such as fenofibrate.

In additional embodiments, the invention is directed to methods of decreasing serum triglyceride levels in a mammal. The methods comprise increasing long-chain fatty acyl-Co-A (LC-CoA) levels in the hypothalamus of the mammal in an amount effective to decrease serum triglyceride levels in a mammal.

As in embodiments described above, the mammal preferably has a condition that would benefit from decreases in triglyceride levels. Nonlimiting examples include obesity, type 2 diabetes, type 1 diabetes, leptin resistance, insulin resistance, metabolic syndrome, gonadotropin deficiency, amenorrhea, polycystic ovary syndrome, familial lipoprotein lipase deficiency, coronary heart disease, atherosclerosis, hypopituitarism, hypercholesterolemia, hyperlipidemia, and hypertriglyceridemia. As discussed in PCT Patent Application PCT/US2004/004344, the LC-CoA levels can be increased by decreasing the activity of an LC-CoA-decreasing molecule in the hypothalamus. Examples of LC-CoA-decreasing molecules are camitine palmitoyl transferase 1 (CPT1), malonyl-CoA decarboxylase, camitine acylcarnitine translocase, acyl-CoA dehydrogenase, 2-enoyl-CoA hydratase, 3-hydroxyacyl-CoA dehydrogenase, 3-oxyacyl-CoA thiolase, and acyl-CoA hydrolase. Preferably, the LC-CoA-decreasing molecule is CPT1, most preferably carnitine palmitoyl transferase-1, liver isoform (CPT1L).

The activity of the LC-CoA-decreasing molecule can be decreased by administering a pharmaceutical composition to the brain of the mammal, where the pharmaceutical composition comprises a small molecule capable of decreasing the activity of the LC-CoA-decreasing molecule. The pharmaceutical composition can be administered to the brain of the mammal by direct administration of the composition to the brain. Alternatively, the small molecule is capable of crossing the blood-brain barrier when present in the pharmaceutical composition.

In other embodiments, the activity of the LC-CoA-decreasing molecule is decreased by administering an antibody or antibody fragment comprising an antibody binding site to the brain of the mammal, where the antibody or antibody fragment is capable of binding to the LC-CoA-decreasing molecule to inhibit the activity of the molecule. Alternatively, the activity of the LC-CoA-decreasing molecule can be decreased by administering an inhibitory nucleic acid or mimetic to the brain of the mammal. Nonlimiting examples of inhibitory nucleic acids or mimetics are ribozymes, antisense compounds, aptamers and iRNAs. In preferred embodiments, the inhibitory nucleic acid or mimetic is a ribozyme, e.g., where the LC-CoA-decreasing molecule is CPT1L and the ribozyme comprises the sequence 5′-ACAGCACGCCGCUCUGAUGAGUCCGUGAGGACGAAACCACGUUCUUCGUC-3′.

In other embodiments, the LC-CoA levels are increased by increasing the activity of an LC-CoA-increasing molecule in the hypothalamus. Examples of LC-CoA-increasing molecules are acetyl-CoA carboxylase, fatty acid transporter molecule, acyl-CoA synthetase, carnitine palmitoyl transferase II, and acyl-CoA thioesterase.

In these embodiments, the activity of the LC-CoA-increasing molecule can be increased by administering a pharmaceutical composition to the brain of the mammal, where the pharmaceutical composition comprises a small molecule capable of stimulating production or activity of the LC-CoA-increasing molecule. Here, the pharmaceutical composition can be administered to the brain of the mammal by direct administration to the brain. Alternatively, the pharmaceutical composition is capable of crossing the blood-brain barrier.

The activity of the LC-CoA-increasing molecule can also be increased by administering the molecule or a vector (e.g., a viral vector) encoding the molecule to the hypothalamus, e.g., in a pharmaceutically acceptable carrier. The LC-CoA can also be increased by directly administering LC-CoA to the brain.

Some preferred pharmaceutical compositions, for example those to be administered nasally, are in an aqueous solution. Examples of preferred aqueous solutions are aqueous gels, aqueous suspensions, aqueous microsphere suspensions, aqueous microsphere dispersions, aqueous liposomal dispersions, aqueous micelles of liposomes, aqueous microemulsions, and any combinations of the foregoing.

Other preferred pharmaceutical compositions, including those to be nasally administered, are in a nonaqueous solution. Preferred nonaqueous solutions are nonaqueous gels, nonaqueous suspensions, nonaqueous microsphere suspensions, nonaqueous microsphere dispersions, nonaqueous liposomal dispersions, nonaqueous emulsions, nonaqueous microemulsions, and any combination of the foregoing.

Additional preferred pharmaceutical compositions, e.g., those to be nasally administered, are in a powder formulation. Preferred such powder formulations are simple powder mixtures, micronized powders, powder microspheres, coated powder microspheres, and any combination of the foregoing.

The pharmaceutical compositions of these methods are not narrowly limited to any pH range. Optimal pH ranges for any composition and intended purpose can be established by the skilled artisan by established methods. Where the pharmaceutical composition is to be administered nasally, the preferred pH is in the range from pH 3.5 to pH 7.

Similarly, pharmaceutical compositions of these methods are not narrowly limited to any osmolarity range, which can be determined for any composition and intended purpose without undue experimentation. For nasal administration, the osmolarity of the pharmaceutical composition is preferably in the range from 150 to 550 mOsM.

Additionally, where these methods utilize nasal administration, the pharmaceutical composition is preferably in the form of liquid droplets or solid particles, more preferably where the majority and/or mean size of the liquid droplets or solid particles range in size from 5 microns to 50 microns.

Methods utilizing nasal administration preferably also comprise at least one absorption enhancer. These absorption enhancers most preferably comprise a chelating agent or a fatty acid.

These methods are also expected to be useful in conjunction with any other disease treatment or prophylaxis, including but not limited to administration of a second compound, where the second compound is useful for the prevention or treatment of obesity, type 2 diabetes, type 1 diabetes, leptin resistance, insulin resistance, metabolic syndrome, gonadotropin deficiency, amenorrhea, lactic acidosis, and/or polycystic ovary syndrome, or a complication thereof. Nonlimiting examples of such compounds are insulin, antihyperglycemic agents such as troglitazone or metformin, α-glucosidase inhibitors such as acarbose, rennin-angiotensin system blockers such as ramipril, antihypertensive drugs such as ACE inhibitors, angiotensin receptor blockers, β-blockers, diuretics, calcium channel blockers, anti-platelet agents such as clopidogrel, poly (ADP-ribose) polymerase (PARP) inhibitors such as PJ34, 3 aminobenzamide, 4 amino 1,8 naphthalimide, 6(5H) phenanthridinone, benzamide, INO 1001, and NU1025 (see PCT Patent Application No. PCT/US04/16562, filed May 27, 2004), and lipid management agents such as statins, ezetinbe, niacin, fibric acid derivatives, and bile acid sequestrants such as fenofibrate.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### EXAMPLE 1

### Effect of Manipulation of Hypothalamic Glucose Metabolism and TCA Cycle on Glucose Production and Plasma Glucose Levels

We hypothesize that the hypothalamic sensing of glucose-derived pyruvate is a novel regulator of endogenous glucose production (GP). We have explored this hypothesis in conscious rats and we are investigating the mechanism responsible for the glucose-dependent signal by examining the role of hypothalamic lactate metabolism in regulating hepatic glucose fluxes. The neuronal enzyme, lactic dehydrogenase (LDH), regulates the entry of lactate in the tricarboxylic acids cycle (TCA) via its conversion to pyruvate. We propose that an increase in neuronal pyruvate levels is a hypothalamic signal of nutrient availability. This increase could be generated by either increased availability of glucose or lactate or via modulation of intracellular pyruvate metabolism. To test this hypothesis we asked whether a primary increase in hypothalamic glucose or lactate is sufficient to inhibit GP and whether this effect requires metabolism of lactate to pyruvate and activation of ATP-dependent potassium channels (K_{ATP}).

In order to increase the availability of lactate in the hypothalamus, L-lactate (substrate for LDH) or D-lactate (as a control) were administered in the third cerebral ventricle (ICV). ICV L-lactate but not D-lactate was sufficient to markedly diminish circulating glucose levels (FIG. 2). We next aimed to establish whether the conversion of lactate to pyruvate was required for the potent effects of ICV L-lactate on plasma glucose. Indeed, inhibition of LDH with ICV oxamate completely eliminated the effect of lactate on GP (FIG. 2). Based on these basic observations we next investigate the following issues.

Glucose Production in the Liver is Regulated by a Glucose-Lactate Pathway in the Brain.

Long chain fatty acids in the brain decrease glucose production (GP) by the liver via activation of hypothalamus ATP-sensitive (K_{ATP}) channels. Here we tested whether (A) direct administration of glucose in the third cerebral ventricle can alter glucose production by the liver, and (B) whether this effect requires its metabolism in the TCA cycle.

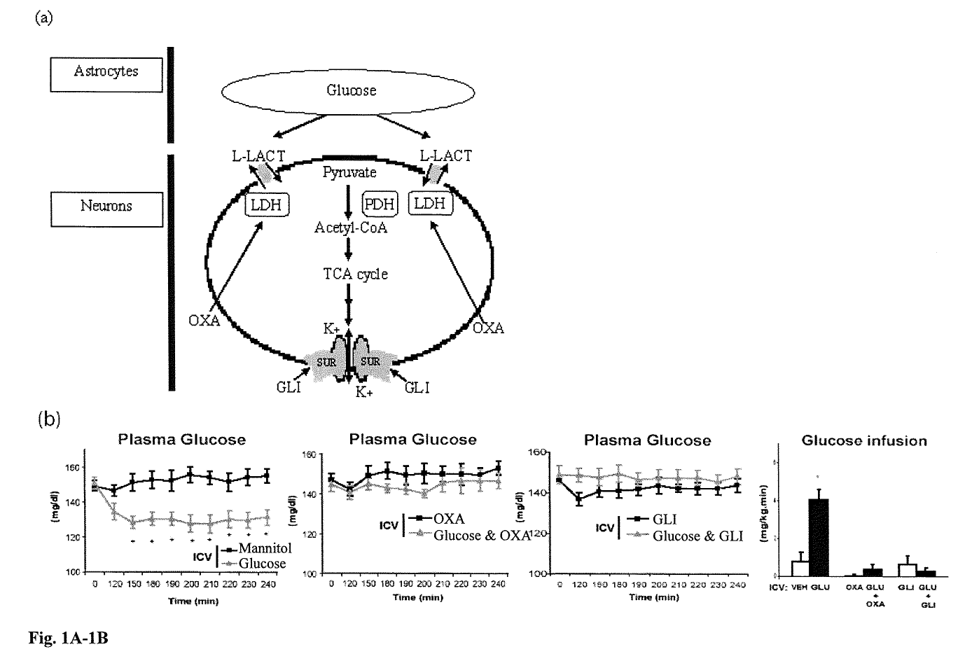

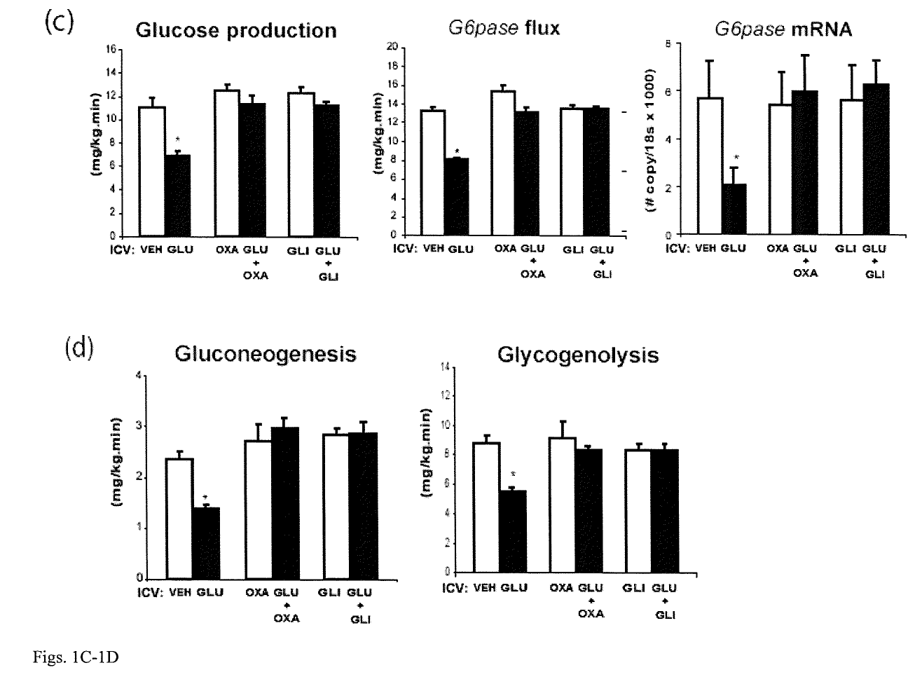

Four hour of ICV glucose (2 mM, 5 μl/h) infusion decreased plasma glucose concentrations from 150±4 mg/dl (control) to 133±5 (P<0.05) (FIG. 1). During the pancreatic clamps when plasma glucose and insulin were maintained at the basal concentrations, ICY glucose decreased GP from 10.4±0.1 mg/kg/min (control) to 7.4±0.3 (P<0.01)(FIG. 1). This indicates that ICV glucose decreases plasma glucose concentrations by decreasing GP in the liver. Inhibition of lactate dehydrogenase by ICY oxamate (50 mM) abolished the effects of ICY glucose on both plasma glucose and GP (11.9±1.0 mg/kg/min), suggesting that lactate metabolism to pyruvate is required for the ICY glucose effects (FIG. 1).

Next, we reasoned that since the metabolism of glucose though glycolysis generates lactate, the direct administration of lactate ICY (5 mM, 5 μl/h) should recapitulate the potent effects of ICV glucose (FIG. 2). Four hour of ICY lactate decreased plasma glucose concentrations from 150±4 mg/dl (control) to 129±3 (P<0.001) and GP from 11.7±0.6 mg/kg/min (control) to 6.5±0.6 (P<0.001), recapitulating the effects of ICY glucose. Similar to ICV glucose, inhibition of lactate dehydrogenase by ICV oxamate prevented the effects of ICY lactate on plasma glucose (144±1 mg/dl) and GP (11.3±0.6 mg/dl). Finally, inhibition of ATP-sensitive K-channels by ICV sulfonylurea administration completely abolished the effects of ICV glucose and lactate on plasma glucose and GP (FIG. 2).

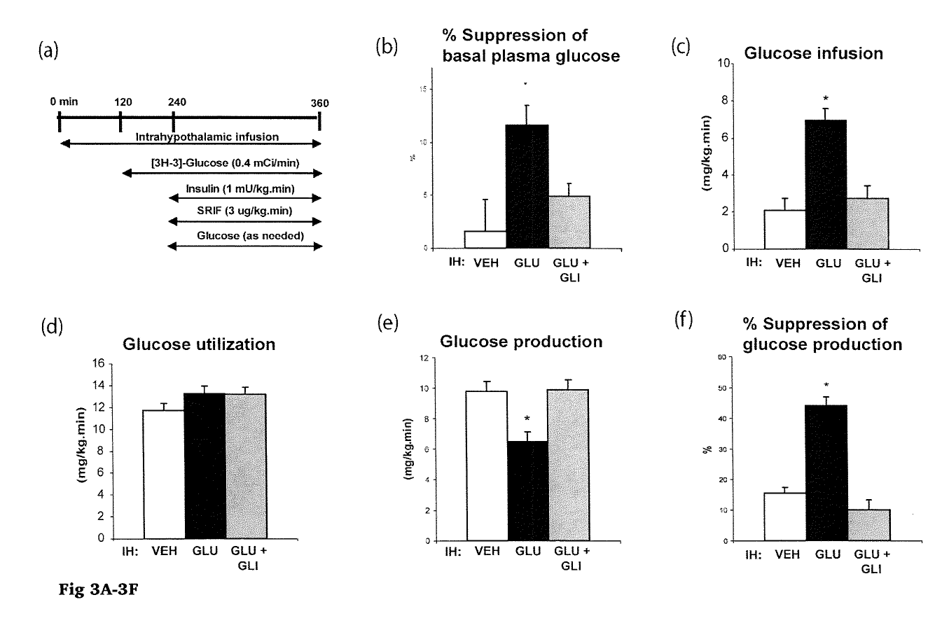

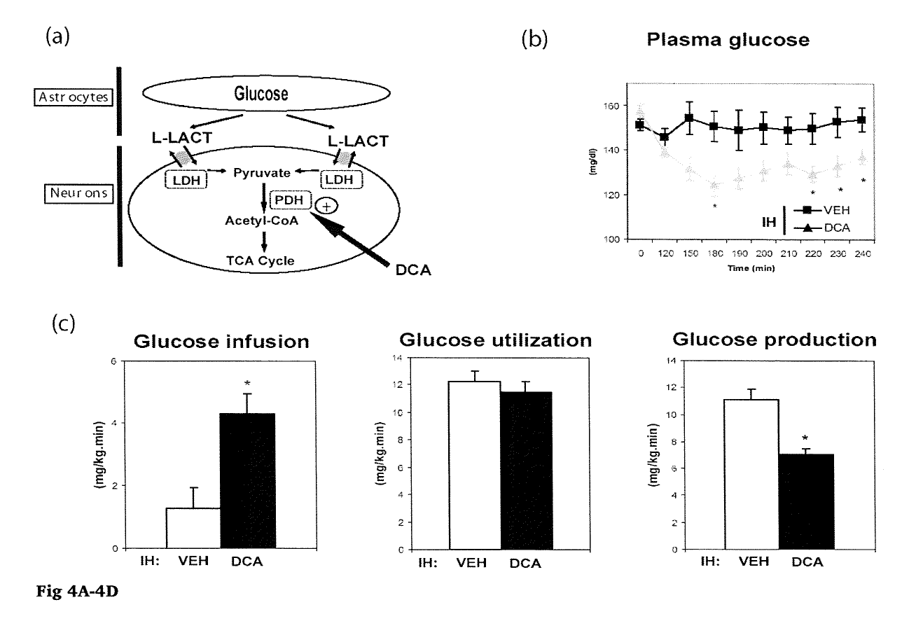

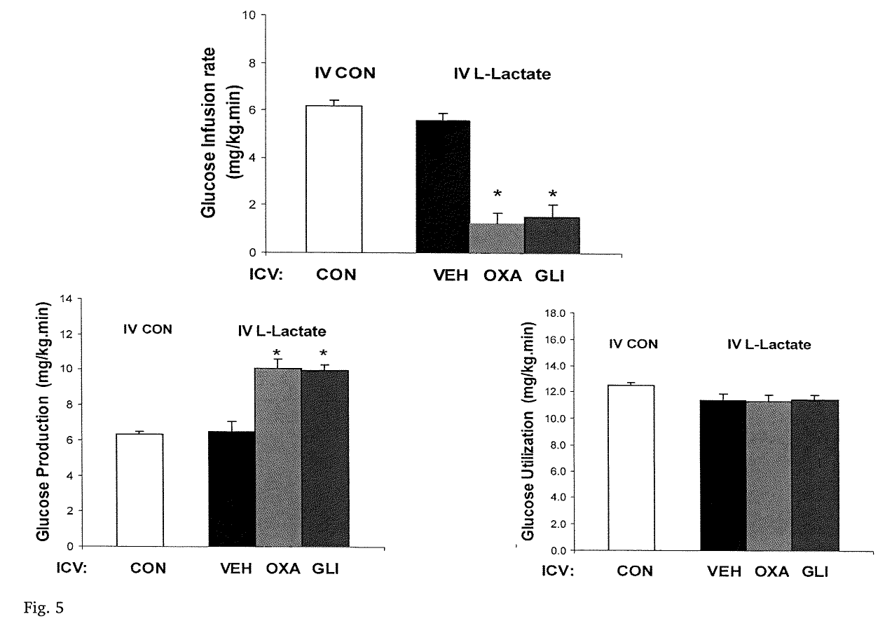

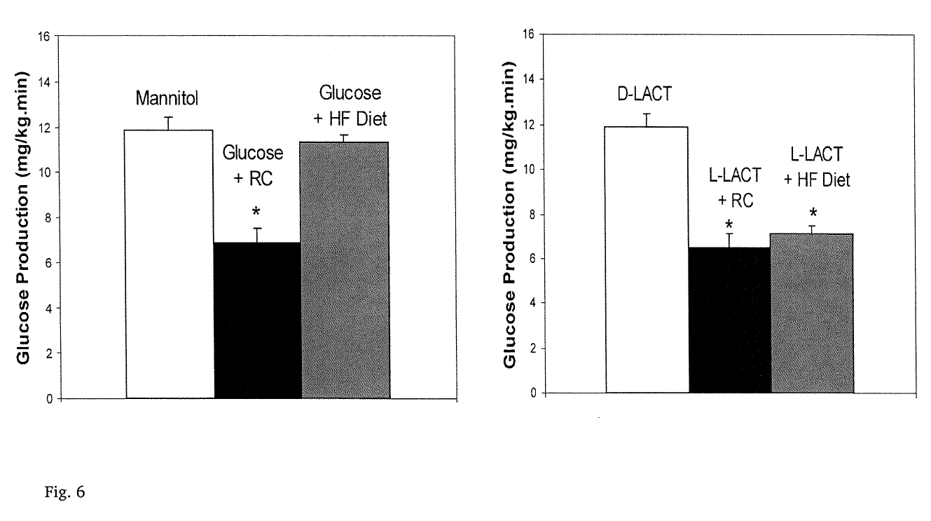

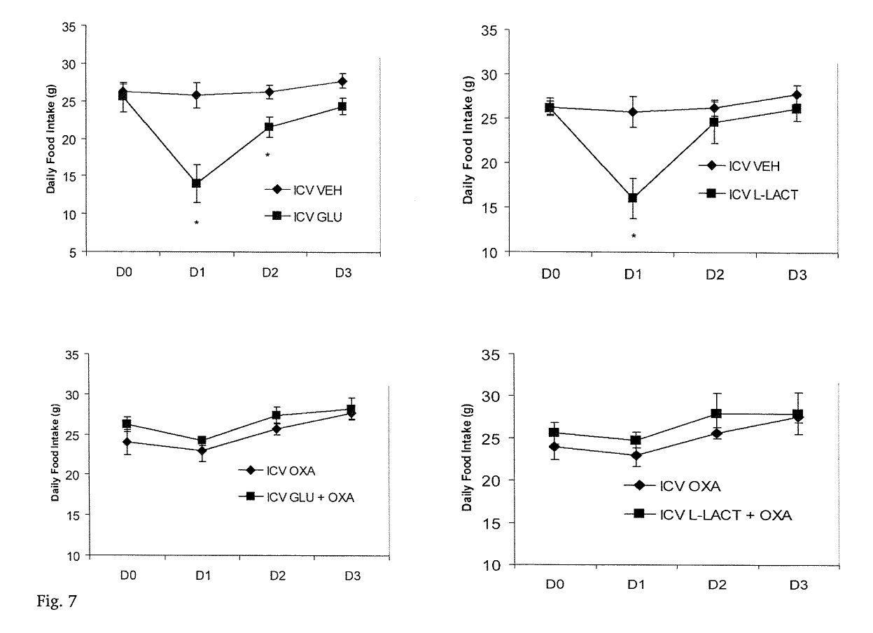

In summary, GP in the liver is normally restrained by glucose in the brain. More importantly, lactate metabolism and activation of K_{ATP} in the brain are required for ICV glucose to decrease plasma glucose concentration and GP. Thus, we postulate that glucose sensing in the brain plays a pivotal role in regulating endogenous GP by the liver and in the maintenance of glucose homeostasis. See FIGS. 3-7 for further supporting data.

In conclusion, this invention disclosure provides strong evidence in support of the notion that increasing the hypothalamic levels of pyruvate will decrease the rate of glucose production and the plasma glucose concentrations. Several alternative strategies to modulate hypothalamic pyruvate levels can be proposed since they represent viable biochemical approaches to the regulation of glucose metabolism via this novel central mechanism.

### EXAMPLE 2

### Effect of Manipulation of Hypothalamic Glucose Metabolism and TCA Cycle on Food Intake

We have examined the effects of ICV glucose and lactate in high fat-fed rats. The latter is an excellent model for diet-induced insulin resistance. The effects of ICV glucose on liver glucose production are severely impaired in this model. Conversely, the effects of ICV lactate are entirely preserved. This set of data suggests that any strategy designed to restore lactate/pyruvate levels within the hypothalamus can overcome the resistance to the central effects of glucose.

Central administration of lactate rapidly inhibits feeding behavior with a ˜40% decrease in food intake the day following its ICV injection. This effect is prevented by the concomitant injection of the LDH inhibitor oxamate.

We have localized the effect of glucose on liver glucose production to the mediobasal hypothalamus. This was accomplished using bilateral intraparenchymal (rather than ICY) delivery of both glucose and the K_{ATP} blocker GLI.

We have also demonstrated that this central ‘lactate/pyruvate’ sensor can be activated by a physiological increase in the circulating levels of lactate. Plasma lactate levels were increased via intracarotid infusion while its hypothalamic metabolism to pyruvate was impeded via ICV or intra-hypothalamic infusions of the LDH inhibitor oxamate. When oxamate was infused centrally the lactate infusion resulted in a significant increase in glucose production.

### EXAMPLE 3

### Brain Glucose Metabolism Controls the Hepatic Secretion of Triglyceride-Rich Lipoproteins

Common Abbreviations: Very Low-Density Lipoprotein, VLDL; ATP-sensitive potassium channel, KATP channel; intracerebroventricular, ICY; Stearoyl-CoA Desaturase-1, SCD-1; Lactic Dehydrogenase, LDH; Microsomal Triglyceride Transfer Protein, MTP; Apolipoprotein B, ApoB.

### Example Summary

Increased production of very low-density lipoprotein (VLDL) is a critical feature of the metabolic syndrome. Reported here is that a primary increase in brain glucose lowers circulating triglycerides through the inhibition of VLDL secretion by the liver. The effect of glucose requires its conversion to lactate leading to activation of ATP-sensitive potassium channels and to decreased hepatic activity of stearoyl-CoA desaturase-1 (SCD-1). SCD1 catalyzes the synthesis of oleyl-CoA from stearoyl-CoA. Curtailing the liver activity of SCD1 is sufficient to lower the hepatic levels of oleyl-CoA and to recapitulate the effects of central glucose on VLDL secretion. Importantly, portal infusion of oleic acid restores hepatic oleyl-CoA to control levels and negates the effects of both central glucose and SCD1 deficiency on VLDL secretion. These central effects of glucose (but not those of lactate) are rapidly lost in diet-induced obesity. These findings indicate that a defect in brain glucose sensing could play a critical role in the etiology of the metabolic syndrome.

### Introduction

A cluster of metabolic risk factors confers susceptibility to type 2 diabetes and cardiovascular disease (Reaven et al., 1993; Ginsberg, 2002; Horton et al., 2002; Reilly and Rader, 2003; Vikramadithyan et al., 2005; Lin et al., 2005; Wolfrum et al., 2004; Flier, 2004). The increased secretion of very low-density lipoprotein (VLDL) by the liver is a key component of this metabolic syndrome (Reaven et al., 1993; Ginsberg, 2002). The biosynthesis and secretion of triglyceride-rich lipoproteins by the liver is a multi-step process that is finely regulated by metabolic and endocrine factors (Horton et al., 2002; Lin et al., 2005; Wolfrum et al., 2004; Lewis et al., 1995; Pan et al., 2002). Substrate availability and insulin play a particularly important role in determining the rate of secretion of VLDL-triglyceride (Lewis et al., 1995). Their influence on this process is accounted for by the regulation of hepatic triglyceride and cholesterol synthesis as well as of the formation and maturation of secreted lipoproteins (Horton et al., 2002; Lin et al., 2005; Wolfrum et al., 2004; Pan et al., 2002; Fisher and Ginsberg, 2002; Wu et al., 1996). During the assembly of nascent VLDL particles with triglyceride droplets Apo B-containing pre-VLDL particles are assembled within the lumen of the rough endoplasmic reticulum through a process partly catalyzed by the microsomal triglyceride transfer protein (MTP) (Fisher and Ginsberg, 2002; Wu et al., 1996). The addition of bulk lipids to the nascent VLDL particle involves the fusion of lipid droplets with pre-VLDL particles and can be stimulated by the availability of the monounsaturated fatty acid oleic acid (Pan et al., 2002). Diets rich in fat and carbohydrates have a profound effect on circulating levels of triglyceride and on VLDL secretion (Lin et al., 2005; Hollenbeck, 1993; Verschoor et al., 1985; Taghibiglou et al., 2000).

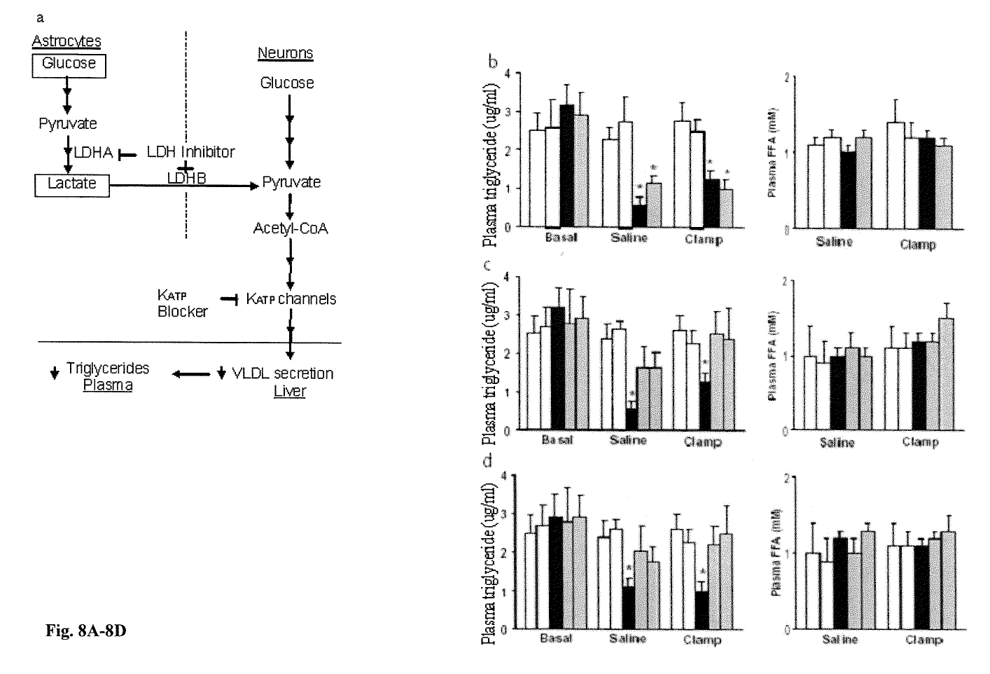

During the last several years experimental evidence has been provided for the notion that the availability of nutrients regulates the activity of hypothalamic neuronal pathways designed to couple energy needs with nutrient intake and endogenous nutrient output (Flier, 2004; Friedman, 2000; Schwartz and Porte, 2005; Lam et al., 2005). These central pathways are prone to faltering in the presence of prolonged over-feeding (Lain et al., 2005a; Howard et al., 2004; Morgan et al., 2004). Brain glucose sensing has been implicated in the regulation of food intake (Davis et al., 1981; Woods and McKay, 1978), hypoglycemia counter-regulation (Borg et al., 1997; Levin et al., 2004), and liver glucose homeostasis (Lam et al., 2005). Here we test the hypothesis that the central metabolism of glucose to pyruvate also controls the rate of VLDL secretion by the liver (FIG. 8a).

### Results

To examine the central effects of glucose on lipid homeostasis, glucose or mannitol (2 mM) was infused into the third cerebral ventricle of conscious rats. Intracerebro-ventricular (ICV) glucose administration raised hypothalamic glucose levels by ˜70% (Lam et al., 2005b) and did not modify plasma fatty acids levels (FIG. 8b). However, it markedly decreased the circulating levels of triglycerides (FIG. 8b). Since changes in circulating insulin levels can modulate triglyceride metabolism (Lewis et al., 1995), we also examined the effect of central glucose on plasma triglyceride levels in the presence of fixed and basal insulin concentrations (pancreatic-basal insulin clamp; Table 2). ICV glucose similarly suppressed plasma triglyceride levels during pancreatic clamp studies (FIG. 8b). Glucose can generate up to two molecules of lactate in astrocytes. Extracellular lactate can then be utilized by neurons for the formation of pyruvate (Pellerin and Magistretti, 2004) (FIG. 8a, ‘astrocyte-neuron lactate shuttle’). Since the effects of central glucose on liver glucose metabolism requires its metabolism to lactate (Lam et al., 2005b), we next investigated whether ICV lactate could recapitulate the effects of central glucose on plasma triglycerides. ICV infusion of L-lactate (5 mM) markedly decreased plasma triglycerides levels (FIG. 8b), but did not affect plasma free fatty acids levels (FIG. 8b). This effect was also observed during pancreatic basal insulin clamp studies (FIG. 8b, Table 3). Thus, a moderate increase in the central availability of glucose or lactate is sufficient to lower plasma triglycerides independent of changes in circulating insulin and free fatty acids levels.

TABLE 2
Characteristics of the groups during the central glucose administration.

| | ICV | ICV | ICV Glucose + | ICV Glucose + |
|---|---|---|---|---|
| | VEH | Glucose | OXA | GLI |
| Basal: | | | | |
| Body weight (g) | 308 ± 4 | 302 ± 7 | 310 ± 5 | 304 ± 8 |
| Lactate (mmol/l) | 0.40 ± 0.10 | 0.39 ± 0.09 | 0.32 ± 0.10 | 0.34 ± 0.09 |
| Insulin (μU/ml) | 28 ± 4 | 14 ± 5* | 29 ± 5 | 24 ± 5 |
| Clamp: | | | | |
| Glucose (mmol/l) | 8.2 ± 0.9 | 8.9 ± 0.5 | 7.9 ± 0.6 | 8.2 ± 0.4 |
| Lactate (mmol/l) | 0.39 ± 0.07 | 0.42 ± 0.07 | 0.41 ± 0.08 | 0.36 ± 0.10 |
| Insulin (μU/ml) | 31 ± 4 | 32 ± 5 | 34 ± 6 | 30 ± 4 |
| Adiponectin (ηg/ml) | 1.3 ± 0.3 | 1.5 ± 0.1 | 1.6 ± 0.2 | 1.4 ± 0.3 |

Data are means ± SEM. The values during the basal and clamp represent steady-state levels obtained by averaging the result of at least five plasma samples during the experimental period. VEH, vehicle (=mannitol/OXA/GLI). OXA, Oxamate. GLI, K_{ATP} channel blocker glibenclamide.
*P < 0.05, ICV VEH vs. ICV Glucose, n = 14, 5, 4, 5 for ICV VEH, ICV Glucose. ICV Glucose + OXA, ICV Glucose + GLI, respectively.

TABLE 3
Characteristics of the groups during central lactate administration.

| | ICV | ICV | ICV L-LACT + | ICV L-LACT + |
|---|---|---|---|---|
| | VEH | L-LACT | OXA | GLI |
| Saline: | | | | |
| Body weight (g) | 309 ± 3 | 309 ± 8 | 306 ± 3 | 313 ± 5 |
| Lactate (mmol/l) | 0.38 ± 0.10 | 0.36 ± 0.08 | 0.31 ± 0.03 | 0.34 ± 0.05 |
| Insulin (μU/ml) | 25 ± 5 | 15 ± 3* | 23 ± 3 | 26 ± 3 |
| Clamp: | | | | |
| Glucose (mmol/l) | 8.5 ± 0.8 | 8.1 ± 0.3 | 8.5 ± 0.6 | 8.1 ± 0.3 |
| Lactate (mmol/l) | 0.45 ± 0.05 | 0.39 ± 0.08 | 0.39 ± 0.02 | 0.40 ± 0.10 |
| Insulin (μU/ml) | 30 ± 5 | 32 ± 7 | 35 ± 5 | 32 ± 6 |
| Adiponectin (ηg/ml) | 1.6 ± 0.2 | 1.1 ± 0.3 | 1.7 ± 0.4 | 1.6 ± 0.4 |

Data are means ± SEM. The values during the basal and clamp represent steady-state levels obtained by averaging the result of at least five plasma samples during the experimental VEH, vehicle (=D-lactate/OXA/GLI). L-LACT, L-Lactate. OXA, Oxamate. GLI, K_{ATP} channel blocker glibenclamide.
*P < 0.05, ICV VEH vs. ICV L-LACT. n = 14, 5, 5, 5 for ICV VEH, ICV L-LACT, ICV L-LACT + OXA, ICV L-LACT + GLI, respectively.

Neurons metabolize lactate to pyruvate through a reaction catalyzed by the enzyme lactic dehydrogenase (mainly LDH-B in neurons). If the hypolipidemic effects of central glucose require either its conversion to lactate or the neuronal utilization of lactate, then the inhibition of LDH should negate the central effects of both glucose and lactate on plasma triglycerides (FIG. 8a). The co-infusion of a competitive inhibitor of LDH abolished the effects of ICV glucose (FIG. 8c) and lactate (FIG. 8d) on plasma triglycerides indicating that the metabolism of lactate to pyruvate is a required biochemical step for the regulation of triglyceride metabolism by central glucose and lactate.

The activation of ATP-sensitive potassium (K_{ATP}) channels within the central nervous system (CNS) is critical for the regulation of blood glucose levels (Pocai et al., 2005) and for some of the metabolic effects of glucose (Pellerin and Magistretti, 2004) and other nutrient-dependent signals (Pocai et al., 2005; Obici et al., 2002b; Lam et al., 2005c; Spanswick et al., 1997; Spanswick et al., 2000). Thus, the role of central K_{ATP} channels in the regulation of circulating triglycerides levels by CNS glucose/lactate sensing waw explored. The K_{ATP} blocker glibenclamide (100 μM) was co-infused ICV with glucose or lactate (FIG. 8a). The central administration of this K_{ATP} blocker did not per se alter the levels of plasma triglycerides but negated the effects of both ICV glucose and lactate on plasma triglycerides (FIGS. 8c, d). To examine how rapidly an increase in brain glucose availability results in lowering of plasma triglyceride levels, glucose or mannitol was next infused ICV in a separate cohort of rats. Plasma triglyceride levels were significantly decreased by ˜⅓ within 2 h and the maximal hypolipidemic effect (˜75% decrease) was observed within 3 h (FIG. 8e). Together these findings indicate that the increased availability of glucose stimulates the metabolism of lactate to pyruvate and activates a K_{ATP} channels-dependent pathway within the hypothalamus that is sufficient to rapidly lower the levels of circulating triglycerides.

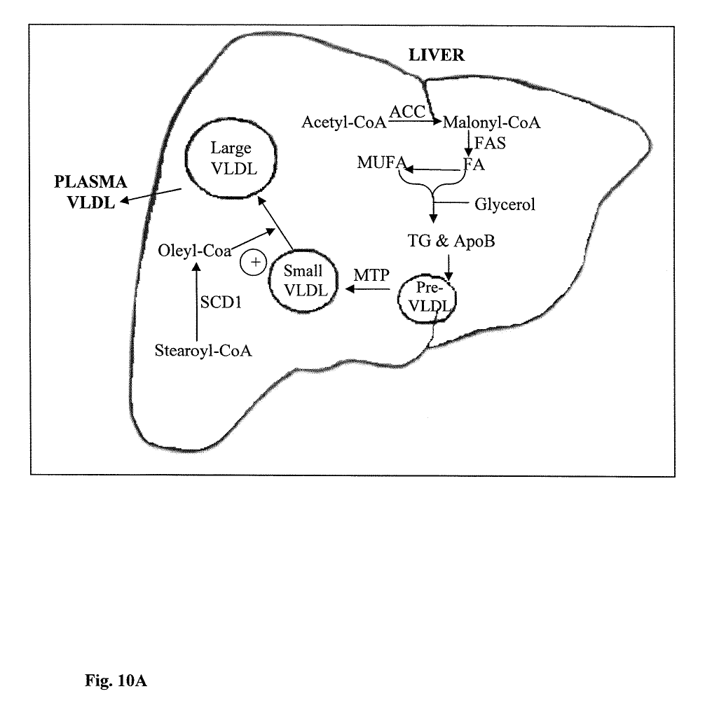

Under post-absorptive conditions, the circulating levels of triglycerides are regulated by the rates of secretion and clearance of triglyceride-rich lipoproteins. To examine whether the hypolipidemic effect of ICV glucose is due to the decreased secretion of triglyceride-rich lipoproteins, we inhibited lipoprotein lipase with tyloxapol (600 mg/kg) (FIG. 9a) and monitored the rate of appearance of triglycerides in plasma as a function of time. ICV glucose suppressed the rate of secretion of VLDL-triglycerides within 90 minutes (FIG. 9a). This effect was abolished by the central co-infusion of the LDH inhibitor or K_{ATP} channels blocker (FIG. 9a). The rate of production and secretion of VLDL particles can reflect changes in the hepatic triglyceride pool (Lin et al., 2005; Wolfrum et al., 2004). Of interest, despite the decrease in plasma triglyceride levels the hepatic content of triglycerides was increased by ICV glucose administration and was restored to normal values by the co-infusion of inhibitors of LDH or K_{ATP} channels (FIG. 9b). These findings indicate that the central administration of glucose lowered plasma triglycerides levels by curtailing the hepatic secretion of triglyceride-rich VLDL rather than by decreasing the hepatic synthesis of triglycerides.

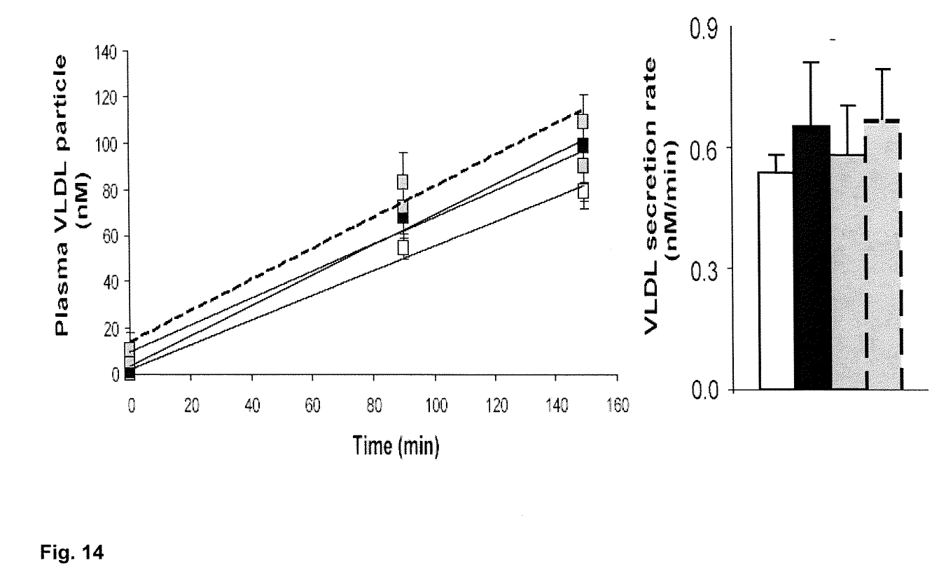

The assembly of nascent VLDL particles with triglyceride droplets is a complex process (Fisher and Ginsberg, 2002) (FIG. 9a). Apo B-containing pre-VLDL particles are assembled within the lumen of the rough endoplasmic reticulum through a process partly catalyzed by the microsomal triglyceride transfer protein (MTP)(Wu et al., 1996). MTP also plays a critical role in the formation of lipid droplets in the smooth endoplasmic reticulum (Fisher and Ginsberg, 2002). The addition of bulk lipids to the nascent VLDL particle involves the fusion of lipid droplets with pre-VLDL particles. This latter MTP-independent step largely determines the extent of lipid transfer to the VLDL particle (and its size) and is stimulated by oleic acid (Pan et al., 2002). To gain insight into potential mechanisms by which central glucose regulates the secretion of triglycerides-enriched lipoproteins we next examined its impact on plasma VLDL particle number and size by nuclear magnetic resonance spectroscopy. Central glucose resulted in a significant decrease in the average size of the secreted VLDL particles (FIG. 9c). Consistent with this finding, ICV glucose dramatically and selectively blunted the secretion of large triglyceride-rich VLDL particles (FIG. 9d; diameter is >60 nm). Conversely, the rate of secretion of small (27-35 nm) and medium (35-60 nm) VLDL particles tended to be higher in response to central glucose and therefore these particles did not contribute to the decreased secretion of VLDL triglycerides (FIG. 14). The central co-infusion of the LDH inhibitor or of K_{ATP} channels blocker (FIGS. 9c, d) negated all the effects of central glucose on circulating VLDLs. To begin investigating the components of this brain-liver circuit we next asked whether the hepatic branch of the vagus nerve is required for the central effects of glucose on VLDL secretion. The inhibitory effect of ICV glucose on VLDL secretion was abolished in rats with resection of the hepatic branch of the vagus but it was preserved in sham-operated rats (FIG. 9e).

To investigate how moderate changes in the central availability of glucose or lactate could impact on the assembly and secretion of VLDL particles, we examined the effects of ICV glucose on the hepatic gene expression of the lipogenic enzymes acetyl-CoA carboxylase and fatty acid synthase. In keeping with its discordant effects on circulating and hepatic triglycerides (FIG. 9b), ICV glucose failed to alter the hepatic content of these lipogenic enzymes (FIG. 9b).

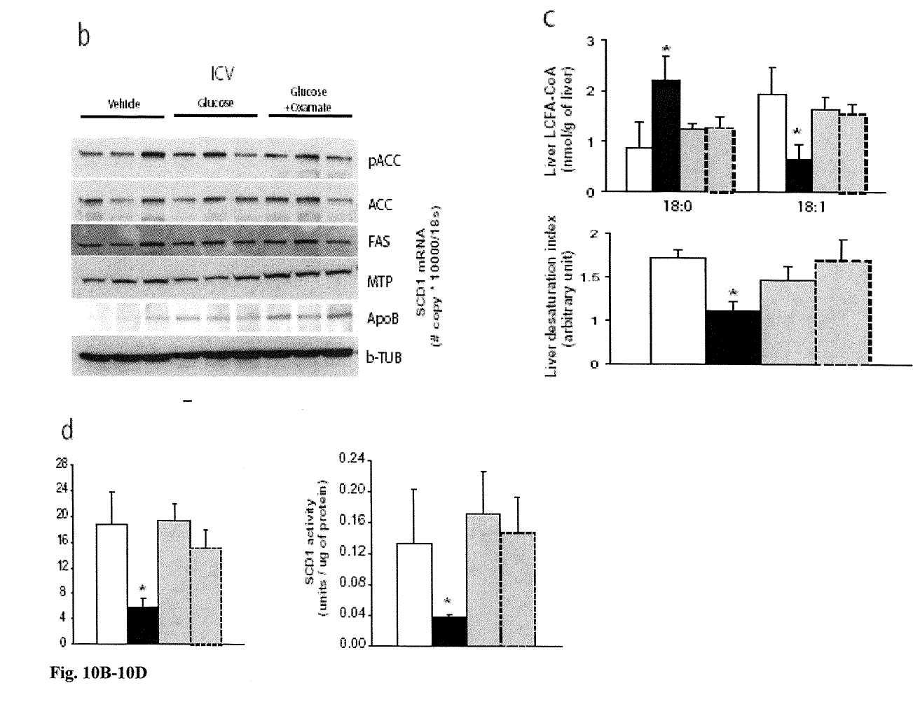

Apolipoprotein B (ApoB) is an essential component of liver-derived VLDL and provides the protein core for the nascent VLDL particle (FIG. 10a). Importantly, a disruption in the early phases of VLDL assembly leads to the targeting of ApoB to the proteasomes for degradation (Fisher and Ginsberg, 2002). Hepatic ApoB48 levels were not decreased by the central administration of glucose suggesting that the inhibition of VLDL secretion is not due to interference with this step (FIG. 10b). MTP plays a pivotal role in multiple steps within lipogenesis and VLDL maturation (Fisher and Ginsberg, 2002) (FIG. 10a). However, the expression of this protein in the liver was not affected by the central administration of glucose (FIG. 10b). Taken together with the selective decrease in the secretion of large VLDL particles in response to ICY glucose, these results point toward central glucose interfering with a late step within the assembling of triglyceride droplets with the nascent VLDL particle. This process appears to be independent of liver triglyceride synthesis and of MTP expression (Wu et al., 1996).

Since oleic acid can dramatically regulate the final steps in the assembly of VLDL particles in hepatocytes (Pan et al., 2002) whether ICV glucose lowered the hepatic levels of oleyl-CoA was next evaluated. Indeed, central glucose decreased the levels of oleyl-CoA while increasing those of stearoyl-CoA in the liver and these effects were dependent on the central activity of LDH and K_{ATP} channels (FIG. 10c). The hepatic levels of other saturated and polyunsaturated acyl-CoAs were not affected (data not shown). Since stearoyl-CoA desaturase-1 (SCD1) is the enzyme responsible for the formation of monounsaturated from saturated acyl-CoAs in the liver, we next assessed the impact of ICV glucose on liver SCD1. The hepatic desaturation index is the ratio of oleyl-CoA over stearoyl-CoA and provides an excellent estimate of the in vivo SCD1 activity. ICV glucose led to a reduction in the hepatic desaturation index that was prevented by the co-infusion of either LDH or K_{ATP} channels antagonists (FIG. 10c). These findings suggest that central glucose restrains the activity of SCD1 in the liver. Consistent with this postulate, ICV glucose markedly decreased the hepatic expression and activity of SCD1 by ˜70% and these effects were negated by the central antagonism of either LDH or K_{ATP} channels (FIG. 10d). Since significant and rapid effects of central glucose on plasma triglyceride within 2 h (FIG. 8e) and on VLDL secretion within 90 minutes (FIG. 9a) was observed, the effects of ICV glucose on liver SCD1 after just 60 minutes of infusion was next examined. Significant decreases in hepatic oleyl-CoA levels, desaturation index, and SCD1 activity were clearly demonstrated after just 1 h of ICV glucose infusion (FIG. 10e). This time course is consistent with a cause-effect relationship between the decrease in SCD1 activity and oleyl-CoA levels and the subsequent decreases in VLDL secretion and plasma triglyceride levels. On the other hand, it is unlikely that such a dramatic and rapid inhibition in the activity of SCD1 could be accounted for by the associated decrease in its hepatic mRNA levels.

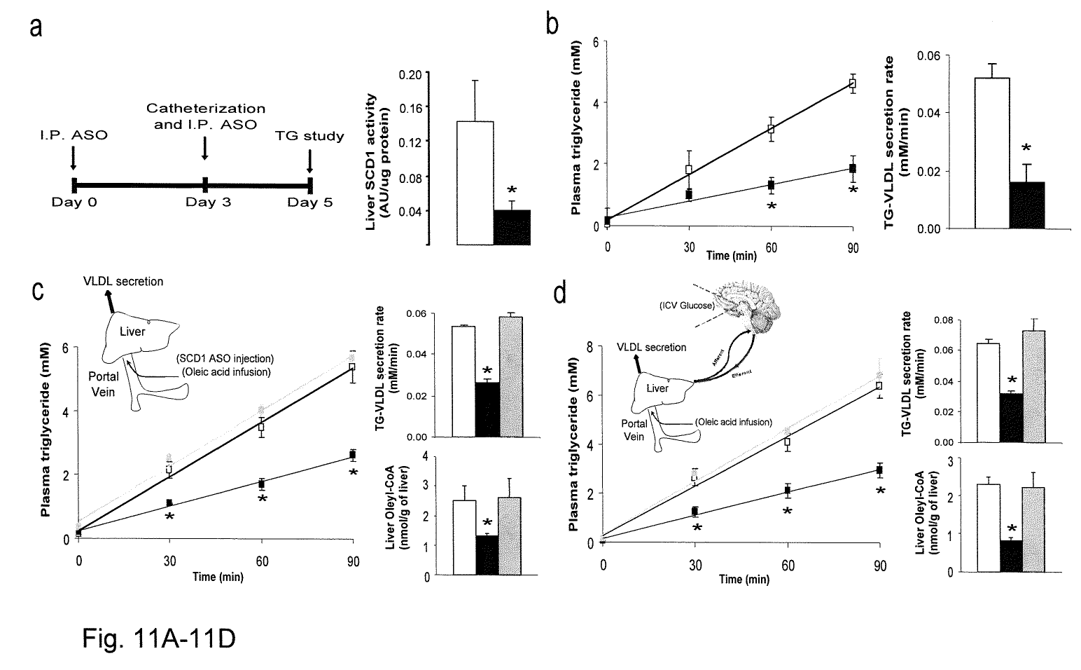

Based on the key role of SCD1 and its product oleyl-CoA in modulating VLDL assembly and secretion we postulated that the acute inhibition of liver SCD-1 activity is largely responsible for the reduction in hepatic oleyl-CoA and that the latter in turn accounts for the rapid decrease in the secretion of large VLDL particles induced by ICV glucose. To test these hypotheses, we used a sequence-specific antisense oligodeoxynucleotide (ASO) to rapidly decrease the hepatic activity of SCD1 (Jiang et al., 2005: Gutierrex-Juarez et al., 2006) to a level comparable to that observed following ICV glucose administration (FIG. 10d). Treatment with the SCD1 ASO reduced the enzymatic activity of SCD1 in the liver by 69% (FIG. 11a). Most important, this reduction was sufficient to reproduce the suppressive effect of ICV glucose on liver VLDL secretion (FIG. 11b). Thus, the inhibitory effect of central glucose on liver triglyceride-rich VLDL could be entirely accounted for by its regulation of liver SCD-1 activity. Since the central administration of glucose as well as a primary decrease in hepatic SCD1 activity lead to similar and marked decreases in the hepatic levels of oleyl-CoA and in VLDL secretion, we reasoned that negating their effects on hepatic oleyl-CoA levels might be sufficient to negate their effect on VLDL secretion. To address this question we implanted chronic portal catheters and then infused oleic acid intra-portally in rats whose hepatic SCD1 activity was curtailed by either SCD1 ASO (FIG. 11c) or by ICV glucose (FIG. 11d). The portal infusion of the SCD1 ASO led to marked decreases in liver oleyl-CoA levels and in the rate of VLDL secretion. These effects were similar to those observed with the intra-peritoneal injection of the same SCD1 ASO. Remarkably the portal infusion of oleic acid at a rate designed to restore the concentration of oleyl-CoA in the liver to that observed in rats receiving the scrambled ASO was sufficient to negate the effect of SCD1 deficiency on VLDL secretion (FIG. 11c). Similarly, negating the effect of ICV glucose on the hepatic levels of oleyl-CoA abolished the effect of central glucose on VLDL secretion (FIG. 11d). These ‘reconstitution’ experiments provide strong support for the pivotal role of hepatic SCD1 and its product oleyl-CoA in mediating the inhibitory action of central glucose on VLDL secretion.

The operation of this brain-liver circuit should normally restrain VLDL secretion particularly in the postprandial state. However, it is increasingly evident that postprandial hyperlipidemia is a common feature in individuals with glucose intolerance and other features of the metabolic syndrome (Ginsberg 2002). Indeed, insulin resistance and obesity are particularly strongly associated with an increase in the secretion of large triglyceride-rich VLDL (diameter >60 nm, VLDL₁) (Gill et al., 2004). Thus, we next examined whether the central effects of glucose on plasma triglyceride and hepatic VLDL were preserved in a model of diet-induced obesity and insulin resistance. Rats fed a palatable lard-enriched diet rapidly increase their daily caloric intake and display hyperlipidemia and severe hepatic insulin resistance (Morgan et al., 2004). In this model (FIG. 12a) the central administration of glucose failed to decrease the circulating levels of triglycerides (FIG. 12b), and the rate of VLDL secretion (FIG. 12c). Thus, a defect in central glucose sensing could partly explain the increased secretion of VLDL and the elevated plasma triglyceride levels in high fat-fed rats. Since lactate is a product of the central metabolism of glucose and its ICV administration also restrains VLDL secretion, we next tested the effect of ICY lactate in this model. ICV lactate effectively lowered the circulating levels of triglycerides to levels similar to those of rats on a standard chow (FIG. 12b). This hypolidemic effect of central lactate was largely due to its inhibition of VLDL secretion (FIG. 12c). Thus, ICV lactate can by-pass the impairment in central glucose sensing indicating that a defect in the conversion of glucose to lactate is rapidly acquired in high fat-fed rats. Most important, this experimental maneuver was sufficient to normalize the circulating lipid levels suggesting that restoring the central sensing of glucose could also improve lipid homeostasis in this model.

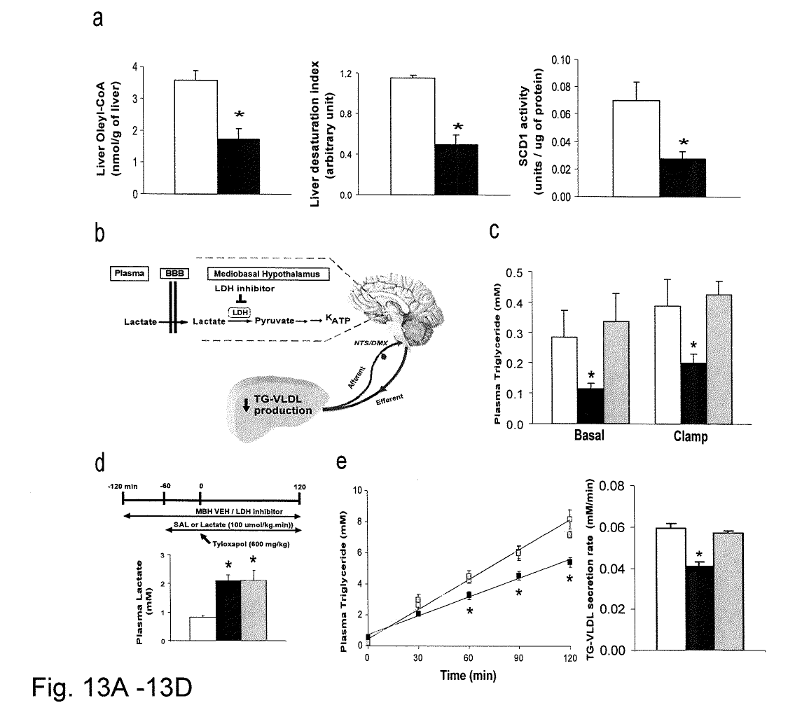

During exercise the plasma lactate concentration rapidly increases to levels that are often several fold (2-5 fold) higher than resting levels. Can a physiological elevation in the circulating levels of lactate recapitulate the effects of central glucose/lactate on liver lipoprotein metabolism? Based on the pivotal role of hepatic oleyl-CoA in the control of VLDL secretion, we first asked whether a systemic lactate infusion is sufficient to inhibit liver SCD1 activity. Indeed, a ˜two-fold increase in circulating lactate levels markedly lowered the hepatic levels of oleyl-CoA as well as the desaturation index and the activity of SCD1 in the liver (FIG. 13a). These findings suggest that increasing the levels of plasma lactate should also curtail VLDL secretion. To this end, a ˜two-fold increase in the circulating lactate levels was first generated and monitored the plasma triglyceride levels under basal and clamp conditions (FIG. 13b). This increase in plasma lactate levels resulted in a significant decrease in plasma triglyceride in both conditions (FIG. 13c). Does the hypolipidemic effect of an increase in plasma lactate require its metabolism within the hypothalamus? If circulating lactate lowers plasma triglyceride through its action within the hypothalamus, then the inhibition of lactate metabolism restricted to this brain area should be sufficient to negate its hypolipidemic effect (FIG. 13b). Since the LDH inhibitor abolished the effects of central glucose and lactate on plasma triglycerides, we infused this inhibitor within the parenchyma of the mediobasal hypothalamus (MBH) in the presence of a physiological elevation in circulating lactate levels (FIG. 13b). MBH infusion of the LDH inhibitor negated the effect of circulating lactate on plasma triglyceride levels (FIG. 13c). To examine whether the effects of circulating lactate on plasma triglyceride levels are also associated with rapid inhibition of VLDL secretion we next increased the circulating lactate levels prior and during VLDL secretion studies (FIG. 13d). A physiological increase in plasma lactate was sufficient to inhibit VLDL secretion and this effect was entirely reversed by the MBH infusion of the LDH inhibitor (FIG. 13e). Thus, the metabolism of circulating lactate within the MBH plays a physiological role in the control of VLDL secretion.

### Discussion

The metabolic syndrome is associated with atherogenic dyslipidemia that includes hypertriglyceridemia and increased production of VLDL by the liver (Reaven et al., 1993; Ginsberg, 2002). We herein provide the first evidence that increased glucose metabolism in the brain lowers circulating triglyceride levels. This cross-talk between the brain and the liver couples carbohydrate sensing to lipoprotein secretion by curtailing the activity of SCD1 in the liver and a late step within the hepatic assembly and secretion of VLDL particles. Oleic acid regulates this step in isolated hepatocytes (Pan et al., 2002) and the activity of SCD1 is a major determinant of the hepatic levels of oleyl-CoA. Consistent with this role of oleic acid/oleyl-CoA in lipoprotein metabolism, the effects of both central glucose and SCD1 deficiency on VLDL secretion keenly depends on the associated decrease in the hepatic levels of oleyl-CoA. Of interest, variations in SCD1 activity (plasma desaturation index) account for a large portion of the variability in plasma triglyceride levels in humans (Attie et al., 2002).

Life-long absence of SCD1 leads to decreases in fat mass, hepatic triglycerides, and plasma VLDL secretion (Miyazaki et al., 2000: Cohen et al., 2002). The rapid effects of central glucose/lactate as well as of the hepatic down-regulation of SCD1 expression selectively reproduced the hypolipidemic phenotype of SCD1 null mice in the absence of a concomitant inhibition of lipid synthesis. It is likely that a more prolonged deficiency in SCD1 expression could also affect multiple steps of liver lipid metabolism and therefore modulate triglyceride metabolism at multiple steps. Since the central effects of glucose on plasma triglyceride levels are rapid (FIG. 8e), we initially examined VLDL secretion during the first 2-3 h following a central infusion of glucose (FIG. 9a). This protocol revealed that a significant decrease in VLDL secretion was detectable within 90 min and therefore preceded the drop in plasma triglyceride levels. Furthermore, the rate of VLDL secretion further declined with longer duration of the ICV glucose infusion (FIG. 11d) in agreement with the progressive decline in circulating triglyceride levels (FIG. 8e). Thus, we postulate that the activation of brain glucose metabolism rapidly curtails the secretion of large triglyceride-rich VLDL particles (FIG. 9d) through its rapid effects on SCD1 activity in the liver (FIG. 10e). The latter effect in turn leads to rapid depletion of oleyl-CoA in the liver and decreased addition of lipid droplets to the nascent VLDL particles (FIGS. 10e, 11c, d).

Our results place SCD1 as the critical target for the effects of brain glucose sensing on VLDL secretion. The regulation of SCD1 appears to include a rapid effect on its enzymatic activity but also a robust effect on SCD1 mRNA. The latter effect is reminiscent of the inhibition of liver SCD1 expression by leptin (Asilmaz et al., 2004) and may be important in mediating a long-lasting beneficial effect on lipoprotein metabolism.

Increased VLDL secretion is a feature of the metabolic syndrome in humans. However, the effect of high fat feeding is more controversial in rodent models. The large variability of the effects reported in rodents are likely due to differing levels of daily caloric intake, length of the dietary intervention, nutritional status (physiological fasting or starvation) and background strain, and particularly to the daily intake of carbohydrates and fat (Lin et al., 2005; Taghibiglou et al., 2000; Gutierrez-Juarez et al., 2006; Kalopissis et al., 1981). Here we observed increased levels of circulating triglycerides and increased VLDL secretion in rats over-fed on a diet rich in both carbohydrates and saturated and monounsaturated fat (lard-enriched; Table 4). Most important, the central administration of lactate but not that of glucose normalized the plasma triglyceride levels and the rate of VLDL secretion in this model.

SUPPLEMENTARY TABLE 3
Diet Composition

| | | SC Diet with |
|---|---|---|
| | Standard Chow | 10% Lard* |
| Calories provided | Diet (SC) | (High Fat Diet) |
| Carbohydrate (%) | 60 | 45 |
| Protein (%) | 28 | 22 |
| Fat (%) | 12 | 33 |
| Saturated | 3.1 | 9.4 |
| Monounsaturated | 4.7 | 11.2 |
| Polyunsaturated | 5.0 | 4.9 |
| Total Calorie Provided by | 3.00 | 5.14 |
| Digestible Nutrients (Kcal/g) | | |

*Lard composition: 2% myristic acid, 24% palmitic acid, 13% stearic acid, 46% oleic acid, 12% linoleic acid.

A physiological increase in circulating lactate levels also decreased plasma triglyceride levels and VLDL secretion and this effect required the metabolism of lactate within the MBH. This experiment indicates that a primary increase in circulating lactate levels similar to that elicited by moderately intense exercise is sufficient to lower VLDL secretion through a central mechanism. Thus the central effect of lactate on VLDL secretion may also be germane to the beneficial effect of exercise on lipoprotein metabolism (Kraus et al., 2002). Furthermore, the central effect of lactate is keenly dependent on its metabolism to pyruvate within the MBH.

These findings are consistent with a homeostatic loop by which the increased availability of carbohydrates limits the endogenous output of lipids into the circulation. Based on our time course studies it is suggested that the central effects of glucose and lactate on VLDL secretion could play a role in restraining hepatic triglyceride output during the hours following a meal or a bout of exercise. This effect may be important in buffering the direct effects of nutrients on the liver (Lewis et al., 1995; Taghibiglou et al., 2000). The impairment in brain glucose sensing is expected to have its major impact on the late postprandial rates of VLDL secretion that are commonly elevated in individuals with obesity and the metabolic syndrome. In light of the fact that changes in brain glucose metabolism also control food intake and hepatic glucose production (Davis et al., 1981; Lam et al., 2005b), we postulate that a defect in this negative feedback could account for multiple components of the metabolic syndrome including obesity, hepatic insulin resistance and hypertriglyceridemia.

### Methods

Animal preparation. We studied 10-week-old male Sprague-Dawley rats. Rats underwent stereotaxic surgery to indwell single catheters in the third cerebral ventricle {intracerebroventricular (ICV)(Obici et al., 2001) or bilateral catheters into the mediobasal hypothalamus (Lam et al., 2005c). One week later, we placed indwelling catheters in the internal jugular vein and carotid artery for sampling and infusion during the in vivo experiments (Obici et al., 2001). Recovery from surgery was monitored by measuring daily food intake and weight gain for 4-5 days after surgery.

Infusion protocols. The in vivo infusion experiments lasted a total of 360 min, and the experiments were carried out in rats that had their food removed ˜5 h before the experiments. Thus, at the time of the experiment, the rats are in post-absorptive state since their stomach should be completely emptied by 5 h fasting. We infused (a) ICV (5 μl/h) 2 mM D-glucose/mannitol and (b) ICV 4-5 mM L-lactate/D-lactate throughout the experiments. This ICV glucose protocol physiologically elevate hypothalamic glucose concentration by ˜70% as previously described (Lam et al., 2005b) and is similar to that induced by a doubling of the circulating glucose levels (Lam et al., 2005b). During the ICV infusions, we monitored the plasma metabolite levels under pre-infused (0 min), basal (180-240 min) and pancreatic clamp (300-360 min) conditions. Lactate dehydrogenase inhibitor oxamate, which was dissolved in artificial cerebrospinal fluid (ACSF) to a final concentration of 50 mM, was first given as an ICY bolus (3 μl). After 30 min, ICV oxamate was co-infused with glucose or L-lactate. K_{ATP} channels blocker glibenclamide, which was dissolved in 5% DMSO to a final concentration of 100 μM, was co-infused with glucose or L-lactate. A basal insulin-pancreatic clamp was performed in the final 2 h (240-360 min) of the study as previously described (Lam et al., 2005b). In brief, a continuous infusion of insulin (1 mU/kg.min) and somatostatin (3 μg/kg.min) was administered, and a variable infusion of a 25% glucose solution was started and periodically adjusted to clamp and maintain the plasma glucose concentration at ˜8 mM. Plasma samples for determination of plasma triglycerides, FFA, insulin, lactate, and adiponectin concentrations were obtained at every 30 min intervals during the study. To examine the time-dependent effects of ICY glucose on plasma triglycerides, ICY (5 μl/h) 2 mM D-glucose or mannitol was administrated for 210 min and plasma triglyceride levels were monitored at 0, 20, 60, 120, 180, 210 min. To examine the hypothalamic effects of circulating lactate on plasma triglycerides, we infused sodium L-lactate (100 μmol/kg.min, pH 7.0) intravenously to elevate plasma lactate to ˜two-fold for 4 h in the presence or absence of intrahypothalamic (mediobasal hypothalamus) LDH inhibitor oxamate. Plasma triglycerides were monitored under basal, pancreatic clamp and tyloxapol-treated conditions. At the end of the infusion experiments, rats were anesthetized and tissue samples were freeze-clamped in situ with aluminum tongs pre-cooled in liquid nitrogen. All tissue samples were stored at −80° C. for subsequent analysis.

Biochemical analyses. Plasma glucose concentrations were measured by the glucose oxidase method (Glucose Analyzer II, Beckman Instruments, Inc., Fullerton, Calif.). Plasma triglycerides and lactate concentrations were determined by a kit in accordance with the manufacturer's instructions (Sigma Diagnostics. St. Louis, Mo.). FFA concentrations were determined by an enzyme assay in accordance with the manufacturer's specifications (Waco Pure Chemical Industries. Osaka. Japan). Plasma insulin concentrations (with rat and porcine insulin standards) were measured by radioimmunoassay (RIA). Plasma adiponectin concentrations were measured by RIA (Linco Inc., Austin, Tex., USA). Liver triglycerides were measured as described (Muse et al., 2004).

Lipoproteins secretion and particles concentration and size measurements. Tyloxapol (600 mg/kg; ˜1 min) was intravenously injected to inhibit endogenous lipoprotein lipase. This high dose of tyloxapol ensured complete inhibition of triglyceride clearance during VLDL secretion studies. Plasma samples were taken every 30 min for triglycerides analysis and for lipoprotein profiling. Plasma VLDL particles concentration and size were determined by nuclear magnetic resonance spectroscopy (lipoprofile-II test) at Liposcience as previously described (Otvos et al., 1991; 1992). This NMR lipoprofile-II test is based on the assumption that each plasma lipoprotein particle of a particular size gives its own characteristic lipid NMR signal. The intensity of the signal is also in proportion to the concentration of the particle. The diameter range for large, medium and small VLDL is >60 nm, 35-60 nm, and 27-35 nm, respectively.

Selective hepatic branch vagotomy. The surgeries were performed as described (Lam et al., 2005c).

Treatment with SCD1 ASO. Five and two days before the lipoprotein secretion studies, rats received a single intra-peritoneal injection of either SCD1 or scrambled (SCR) ASO (100 mg/kg of body weight). A dose-titration effect of SCD1 ASO (25, 50 and 100 mg/kg) on SCD1 activity was previously performed (Gutierrez-Juarez et al., 2006) and we found that 100 mg/kg consistently achieved the largest (˜70%) down-regulation of liver SCD1 activity. In a separate set of studies, rats received intra-portal injection of either SCD1 or SCR ASO (40 mg/kg of body weight) (Gutierrez-Juarez et al., 2006) three days before the lipoprotein secretion studies.

Portal oleate infusion. Chronic portal catheter was implanted and 7 days were allowed for rat recovery. Oleic acid (45 mM; dissolved in 45% HBP) was infused at a rate of 40 μl/h for 2.5 h (1 h before the 90 min lipoprotein secretion studies). This dose enabled the reconstitution of hepatic oleyl-CoA levels back to normal during portal SCD1 ASO (FIG. 11c) or ICV glucose studies (FIG. 11d).

High fat feeding. Male Sprague-Dawley rats were fed a highly palatable high fat diet for three days. These rats developed hyperinsulinemia and hepatic insulin resistance (Morgan et al., 2004). The rats were then subjected to two hour ICV vehicle, glucose or lactate administrations, and plasma triglycerides were monitored. In a separate set of studies, lipoprotein secretion studies were performed at the beginning of the ICV infusions.

Liver LCFA-CoA measurements. The liver was sampled and the LCFA-CoAs were extracted from the liver and measured by high-performance liquid chromatography (HPLC) as previously described (Lam et al., 2005c).

Stearoyl-CoA Desaturase 1 (SCD1) activity. CD1 activity assays were performed essentially as described (Cohen et al., 2002) using 1-[¹⁴C]-stearoyl-CoA as a substrate. At the end of the reaction incubation long-chain acyl CoAs (LC Acyl-CoAs) were extracted and fractionated by reverse-phase HPLC as previously reported (Lam et al., 2005c). Chromatographically resolved stearoyl CoA (C18:0) and oleyl-CoA (C18:1) peaks were collected and counted in a β-scintillation counter. Protein was assayed by the bicinchoninic acid (BCA) method using a commercially available kit (Pierce Biotechnology, Rockford, Ill.) according to the manufacturer's instructions.

Gene expression. Stearoyl-CoA Desaturasel (SCD1) mRNA was measured by quantitative real-time PCR. Briefly, total RNA was extracted from either liver or fat tissue using the Trizol reagent (Invitrogen, Carlsbad, Calif.) according to the manufacturer instructions. cDNA was then synthesized from 2 μg of total RNA using the Superscript® III First-Strand kit TM? (Invitrogen, Carlsbad, Calif.). Samples of RNA in which the reverse transcriptase was omitted (no RT controls) were also included. The cDNA and no RT controls were diluted 1:20 with PCR grade water and 2 μl from each sample was used for PCR in a total volume of 20 μl. The FastStart DNA Master SYBR Green I kit (Roche Applied Science, Indianapolis, Ind.) and a LightCycler® 2 instrument (Roche Applied Science, Indianapolis, Ind.) were used in all experiments. Either 18S mRNA expression was routinely measured for normalization purposes. The primer sequences used were as follows: 5′-CTACAAGCCTGGCCTCCTGC-3′ and 5′-GGACCCCAGGGAAACCAGGA-3′ for Scd1; and 5′-AGGGTTCGATTCCGGAGAGG-3′ and 5′-CAACTTTAATATACGCTATTGG-3′ for ribosomal 18S. The mRNA copy number was calculated from standard curves obtained using cloned target templates (plasmid DNA) of known copy number for each gene of interest.

Western blot analyses. Liver tissue (50-100 mg) was homogenized in a detergent-based lysis buffer (M-PER, Pierce) supplemented with protease and phosphatase inhibitors. The extracts were incubated on ice for 20 min and then centrifuged at 15,000×g to remove tissue debris; the supernatants were saved and frozen at −80 C until analysis. Fifty μg of protein were heat-denatured in SDS-PAGE sample buffer and resolved on denaturing (SDS) 10% polyacrylamide gels (SDS-PAGE). Fractionated proteins were electrophoretically transferred to nitrocellulose membranes by standard procedures. Membranes were immunoblotted with the appropriate primary antibodies and immune complexes were detected by enhanced chemiluminescence (ECL) (Western Lightning, Perkin Elmer Life Sciences) using horseradish peroxidase conjugated secondary antibodies. Bands were imaged on X-ray film and digitized for quantification using ImageQuant software (Molecular Dynamics). β-tubulin was used for loading normalization.

Antibodies. Rabbit antibodies to acetyl Co-A carboxylase and phospho-acetyl Co-A carboxylase (Ser79) were from Cell Signaling Technology (Beverly, Mass.); fatty acid synthetase (FAS) and microsomal triglyceride transfer protein (MTP) antibodies were from BD Biosciences Pharmingen (San Jose, Calif.); cholesterol hydroxylase 7α (CYP7A1) and ApoB-100 antibodies were from Santa Cruz Biotechnology (Santa Cruz, Calif.); mouse monoclonal β-tubulin antibodies were obtained from Covance Research Products (Berkeley, Calif.).

Calculations. Statistical analysis was done by unpaired Student's t test or analysis of variance as appropriate. Data are presented as means±SEM. All values were collapsed into a single value for each animal prior to the calculation of mean. The time period 180-240 min was averaged for the basal and the time period 300-360 min was averaged for the clamp conditions.

### EXAMPLE 4

### Exercise May Decrease Plasma Triglycerides by Triggering Brain Lactate Sensing

It is well established that the circulating levels of triglyceride (TG) and TG-enriched

VLDL are decreased in the hours following an acute bout of exercise. Increased central metabolism of lactate to pyruvate via lactate dehydrogenase (LDH) suppresses plasma TG levels (by ˜50%) and hepatic TG-enriched VLDL production (by ˜40%) in rats (Example 3). Investigated here is whether a physiological increase in circulating lactate per se can regulate plasma TG levels and hepatic VLDL production in conscious rats. Moderate-intensity treadmill exercise (16 m•min-1, 15% grade) for 2 h resulted in an immediate and sustained 2-fold increase in plasma lactate levels in male Sprague-Dawley rats (n=5). A separate cohort of rats next received an intravenous (i.v.) infusion of lactate (100 μmol•kg-1•min-1, pH 6.8) designed to reproduce this increase in lactate levels in the absence of exercise. This elevation of plasma lactate was sufficient to lower plasma TG levels by >50% (from 0.28±0.09 with i.v. saline, n=4, to 0.11±0.02 mM, n=6; p<0.05). To investigate the mechanism by which lactate lowers plasma TG levels, the rate of production of hepatic TG-enriched VLDL was measured. VLDL production was decreased by 31% (from 0.059±0.003 with i.v. saline, n=4, to 0.041±0.002 mM•min⁻¹, n=5, p<0.01) during the lactate infusion. Strikingly, the direct administration of the LDH inhibitor, oxamate, within the mediobasal hypothalamus prior to the lactate infusion completely abolished the TG lowering effects of circulating lactate (0.44±0.12 mM, n=4) as well as the restraint on hepatic TG-enriched VLDL production (0.056±0.002 mM•min⁻¹, n=5). These findings indicate that the hypothalamus can sense increases in circulating lactate similar to those seen during exercise. The central conversion of lactate to pyruvate in turn activates a brain-liver circuit that curtails the hepatic TG-enriched VLDL production leading to lower plasma TG levels. Our findings suggest a novel mechanism for the beneficial effects of exercise on circulating TG, a key risk factor to the metabolic syndrome.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

1. A method of reducing glucose production or food intake or peripheral blood glucose levels in a mammal, the method comprising administering a compound to the mammal, wherein administering the compound to the mammal causes an increase in tricarboxylic acid (TCA) cycle flux through acetyl-CoA in the hypothalamus of the mammal and a reduction in glucose production or food intake or peripheral blood glucose levels in the mammal.

2. The method of claim 1, wherein the mammal has at least one condition, wherein the condition is obesity, type 2 diabetes, type 1 diabetes, leptin resistance, insulin resistance, metabolic syndrome, gonadotropin deficiency, amenorrhea, lactic acidosis, or polycystic ovary syndrome.

3. (canceled)

4. The method of claim 1, wherein the compound is pyruvate or a metabolic precursor of pyruvate.

5. The method of claim 4, wherein the compound is lactate, a monosaccharide, a disaccharide or an oligosaccharide.

6. The method of claim 4, wherein the compound is lactate.

7. The method of claim 4, wherein the compound is glucose.

8-14. (canceled)

15. The method of claim 12, wherein the compound is a small

16. The method of claim 13, wherein the small organic molecule inhibitor of PDHK is dichloroacetic acid, chlorofluoroacetic acid, difluoroacetic acid, AZD7545, an anilide derivative of (R)-3,3,3-trifluoro-2-hydroxy-2-methylpropionic acid, or a secondary amine of (R)-3,3,3-trifluoro-2-hydroxy-2-methylpropionic acid.

17. The method of claim 15, wherein the compound is an inner lipoyl domain of dihydrolipoyl acetyltransferase.

18. The method of claim 1, wherein the compound is administered directly to the brain of the mammal.

19. The method of claim 1, wherein the compound is formulated in a pharmaceutically acceptable carrier to form a pharmaceutical composition that enhances the ability of the compound to cross the blood-brain barrier of the mammal.

20-33. (canceled)

34. The method of claim 1, wherein the mammal is a human.

35-70. (canceled)

71. A method of inhibiting gluconeogenesis in the liver of a mammal, the method comprising administering a compound to the mammal, wherein administering the compound to the mammal causes an increase in tricarboxylic acid (TCA) cycle flux through acetyl-CoA in the hypothalamus of the mammal and an inhibition of gluconeogenesis in the mammal.

72-127. (canceled)

128. A method of decreasing serum triglyceride levels or very low density lipoprotein (VLDL) levels in a mammal, the method comprising administering a compound to the mammal, wherein administering the compound to the mammal causes an increase in tricarboxylic acid (TCA) cycle flux through acetyl-CoA in the hypothalamus of the mammal and a decrease in serum triglyceride levels or VLDL levels in the mammal.

129-197. (canceled)

198. A method of increasing glucose production or food intake in a mammal, the method comprising administering a compound to the mammal, wherein administering the compound to the mammal causes a decrease in tricarboxylic acid (TCA) cycle flux through acetyl-CoA in the hypothalamus of the mammal and an increase in glucose production or food intake in the mammal.

199-249. (canceled)

250. A method of decreasing very low density lipoprotein (VLDL) levels or serum triglyceride levels in a mammal, the method comprising increasing long-chain fatty acyl-Co-A (LC-CoA) levels in the hypothalamus of the mammal in an amount effective, to reduce VLDL levels or serum triglyceride levels in the mammal.

251-306. (canceled)
